(19) United States
(12) Patent Application Publication
(10) Pub. No.: US 2025/0340874 A1
(43) Pub. Date: Nov. 6, 2025

(54) COMPOUNDS AND METHODS FOR REDUCING PCDH19 EXPRESSION

(71) Applicant: Ionis Pharmaceuticals, Inc., Carlsbad, CA (US)

(72) Inventors: Ruben E. Valas, Oceanside, CA (US); Susan M. Freier, San Diego, CA (US); Jacqueline G. O'Rourke, Vista, CA (US)

(73) Assignee: Ionis Pharmaceuticals, Inc., Carlsbad, CA (US)

(21) Appl. No.: 18/722,875

(22) PCT Filed: Dec. 21, 2022
(86) PCT No.: PCT/US2022/082164

§ 371 Date: Jun. 21, 2024

Related U.S. Application Data
(60) Provisional application No. 63/292,829, filed on Dec. 22, 2021.

(51) Int. Cl. C12N 15/113

(52) U.S. Cl. CPC C12N 15/113; C12N 2310/11; C12N 2310/14; C12N 2310/315; C12N 2310/321; C12N 2310/322; C12N 2310/3231; C12N 2310/341; C12N 2310/345

(57) ABSTRACT

Provided are oligomeric agents, oligomeric compounds, methods, and pharmaceutical compositions for reducing the amount or activity of Protocadherin 19 (PCDH19) RNA in a cell or subject, and in certain instances reducing the amount of PCDH19 protein in a cell or subject. Such oligomeric agents, oligomeric compounds, methods, and pharmaceutical compositions are useful to ameliorate at least one symptom or hallmark of a neurodevelopmental disease or disorder. Such neurodevelopmental diseases or disorders include PCDH19 Epilepsy. Such symptoms or hallmarks include seizures, cognitive impairment, intellectual disabilities, autism spectrum disorder, behavioral problems, aggression, anxiety, obsessive-compulsive disorder, hyperactivity, attention deficit disorder (ADD), and attention deficit hyperactivity disorder (ADHD).

### SEQUENCE LISTING

The present application is being filed along with a Sequence Listing in electronic format. The Sequence Listing is provided as a file entitled BIOL0443SEQ.xml, created on Dec. 16, 2022, which is 1.37 MB in size. The information in the electronic format of the sequence listing is incorporated herein by reference in its entirety.

### FIELD

Provided are oligomeric agents, oligomeric compounds, methods, and pharmaceutical compositions for reducing the amount or activity of Protocadherin 19 (PCDH19) RNA in a cell or subject, and in certain instances reducing the amount of PCDH19 protein in a cell or subject. Such oligomeric agents, oligomeric compounds, methods, and pharmaceutical compositions are useful to ameliorate at least one symptom or hallmark of a neurodevelopmental disease or disorder. Such neurodevelopmental diseases or disorders include PCDH19-Epilepsy. Such symptoms or hallmarks include seizures, cognitive impairment, intellectual disabilities, autism spectrum disorder, behavioral problems, aggression, anxiety, obsessive-compulsive disorder, hyperactivity, attention deficit disorder (ADD), and attention deficit hyperactivity disorder (ADHD).

### BACKGROUND

The X-linked gene encoding Protocadherin 19 (PCDH19) is predominantly expressed in the central nervous system and has been implicated in cell-cell adhesion and synaptic function. PCDH19 mutations lead to PCDH19-associated neurodevelopmental diseases and disorders, including PCDH19-Epilepsy (also known as PCDH19-Girl Clusterng Epilepsy (GCE) Epilepsy, PCDH19 Disorder, and Mental Retardation Limited to Females (EFMR)) (Hoshima, N., et al., 2021, Science, April 16;372 (6539): eaaz3893. doi: 10.1126/science.aaz3893). PCDH19-Epilepsy is the second most common cause of epilepsy; about one in ten girls who have seizures before the age of five have PCDH19-Epilepsy. PCDH19-Epilepsy has a unique pattern of inheritance, due to random X chromosome inactivation, PCDH19-Epilepsy is associated with mosaic expression of mutant PCDH19. The mutation leads to aberrant neural development and is found in females who are heterozygous for PCDH19 mutations, and males who are mosaic carriers of somatic PCDH19 mutaions. Hemizygous males generally do not experience symptoms or have more subtle phenotypes (Thomas, P., et al., 2018, Neuron, 97, 59-66). PCDH19 mutations are associated with seizures (including clusters of seizures, generalized tonic-clonic and/or focal seizures, which may evolve to bilateral, tonic-clonic seizures), cognitive impairment, intellectual disabilities, autism spectrum disorder, behavioral problems, aggression, anxiety, obsessive-compulsive disorder, hyperactivity, attention deficit disorder (ADD), and attention deficit hyperactivity disorder (ADHD). Upon reaching adolescence, there may be a reduction or remission of seizures, however, one or more of cognitive impairment, intellectual disabilities, autism spectrum disorder, behavioral problems, aggression, anxiety, obsessive-compulsive disorder, hyperactivity, attention deficit disorder (ADD), and attention deficit hyperactivity disorder (ADHD) remain (Kolc., K. L., et al., 2019, Mol. Psych. 24, 241-251; Kolc, K. L., et al., 2020, Transl. Psych. 10, 127).

Currently there is a lack of acceptable options for treating diseases and disorders associated with PCDH19 mutations. It is therefore an objective herein to provide compounds and pharmaceutical compositions for the treatment of such diseases and disorders.

### SUMMARY

Oligomeric agents, oligomeric compounds, and pharmaceutical compositions of certain embodiments described herein are useful for reducing or inhibiting PCDH19 expression in a cell or subject. In certain embodiments, PCDH19 RNA or protein levels can be reduced in a cell or subject. Also provided are methods of treating PCDH19 Epilepsy.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included” is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank, ENSEMBL, and NCBI reference sequence records, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-MOE” means a 2′—OCH₂CH₂OCH₃ group in place of the 2′—OH group of a furanosyl sugar moiety. A “2′-MOE sugar moiety” or a “2′-O-methoxyethyl sugar moiety” means a sugar moiety with a 2′-OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety. As used herein, “2′-OMe” means a 2′—OCH₃ group in place of the 2′—OH group of a furanosyl sugar moiety. A″2′-O-methyl sugar moiety” or “2′-OMe sugar moiety” means a sugar moiety with a 2′—OCH₃ group in place of the 2′—OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-F” means a 2′—F group in place of the 2′—OH group of a furanosyl sugar moiety. A″2′-O-fluoro sugar moiety” or “2′-F sugar moiety” means a sugar moiety with a 2′—OF group in place of the 2′—OH group of a furanosyl sugar moiety. Unless otherwise indicated, a 2′-F sugar moiety is in the β-D-ribosyl configuration.

As used herein, “xylo 2′-F” means a 2′-F sugar moiety in the β-D-xylosyl configuration.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administration” or “administering” means providing a pharmaceutical agent or composition to an animal.

As used herein, “ameliorate” in reference to a disease or condition means improvement in at least one symptom of the disease or condition relative to the same symptom in the absence of the treatment. In certain embodiments, amelioration is the reduction in the severity or frequency of the symptom or the delayed onset or slowing of progression in the severity or frequency of a symptom.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “blunt” or “blunt ended” in reference to an oligomeric duplex formed by two oligonucleotides mean that there are no terminal unpaired nucleotides (i.e. no overhanging nucleotides). One or both ends of an oligomeric duplex can be blunt.

As used herein, “cell-targeting moiety” means a conjugate group or portion of a conjugate group that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (“C) and guanine (G). Certain modified nucleobases that pair with natural nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “double-stranded” in reference to a region or an oligonucleotide, means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides.

As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an oligomeric duplex and a nucleic acid target. In certain embodiments, complementary nucleic acid molecules include, but are not limited to, an antisense oligonucleotide and a nucleic acid target.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

“Lipid nanoparticle” or “LNP” is a vesicle comprising a lipid layer encapsulating a pharmaceutically active molecule, such as a nucleic acid molecule, e.g., an RNAi or a plasmid from which an RNAi is transcribed. LNPs are described in, for example, U.S. Pat. Nos. 6,858,225, 6,815,432, 8,158,601, and 8,058,069, the entire contents of which are hereby incorporated herein by reference.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound, or a fragment of a compound, comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “nucleoside overhang” refers to unpaired nucleotides at either or both ends of a duplex formed by hybridization of two oligonucleotides.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide.

As used herein, an “oligonucleotide duplex” means a duplex formed by two paired oligonucleotides having complementary nucleobase sequences. Each oligo of an oligonucleotide duplex is a “duplexed oligonucleotide” or a “double-stranded oligonucleotide.”

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by an animal. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to an animal. For example, a pharmaceutical composition may comprise an oligomeric compound and a sterile aqueous solution. In certain embodiments, a pharmaceutical composition shows activity in free uptake assay in certain cell lines.

As used herein, “reducing the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “self-complementary” in reference to an oligonucleotide means an oligonucleotide that at least partially hybridizes to itself.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard cell assay” and “standard in vitro assay” are used interchangeably herein and the terms mean the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the(S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. The terms “subject” “animal” and “individual” are used interchangeably. In certain embodiments, the subject is human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing said subject. In certain embodiments, a hallmark is apparent upon invasive diagnostic testing, including, but not limited to, post-mortem tests.

As used herein, “target nucleic acid” and “target RNA” mean a nucleic acid that an antisense compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulation of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

### CERTAIN EMBODIMENTS

The present disclosure provides the following non-limiting numbered embodiments:

Embodiment 1. An oligomeric compound comprising a modified oligonucleotide consisting of 12 to 50 linked nucleosides wherein the nucleobase sequence of the modified oligonucleotide is at least 80% complementary to an equal length portion of an PCDH19 nucleic acid, and wherein the modified oligonucleotide comprises at least one modification selected from a modified sugar and a modified internucleoside linkage.

Embodiment 2. An oligomeric compound comprising a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, or 20 contiguous nucleobases of any of SEQ ID NOs: 15-482, wherein the modified oligonucleotide comprises at least one modification selected from a modified sugar and a modified internucleoside linkage.

Embodiment 3. An oligomeric compound comprising a modified oligonucleotide consisting of 12 to 50 linked nucleosides, wherein the nucleobase sequence of the modified oligonucleotide comprises at least 12, at least 13, at least 14, at least 15, or 16 contiguous nucleobases of any of SEQ ID NOs: 15-560, wherein the modified oligonucleotide comprises at least one modification selected from a modified sugar and a modified internucleoside linkage.

Embodiment 4. The oligomeric compound of embodiment 2 or embodiment 3, wherein the modified oligonucleotide has a nucleobase sequence consisting of the nucleobase sequence of any of SEQ ID NOs: 15-560.

Embodiment 5. An oligomeric compound comprising a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, at least 20, at least 21, at least 22, or at least 23 contiguous nucleobases of any of SEQ ID NOs: 561-1028, wherein the modified oligonucleotide comprises at least one modification selected from a modified sugar and a modified internucleoside linkage.

Embodiment 6. The oligomeric compound of embodiment 5, wherein the modified oligonucleotide has a nucleobase sequence consisting of the nucleobase sequence of any of SEQ ID NOs: 561-1028.

Embodiment 7. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases complementary to: an equal length portion of nucleobases 4,743-4,767 of SEQ ID NO: 1;
an equal length portion of nucleobases 12,319-12,346 of SEQ ID NO: 1;
an equal length portion of nucleobases 34,364-34,389 of SEQ ID NO: 1; or
an equal length portion of nucleobases 84,408-84,431 of SEQ ID NO: 1;
wherein the modified oligonucleotide comprises at least one modification selected from a modified sugar and a modified internucleoside linkage.

Embodiment 8. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases of a sequence selected from:
SEQ ID NO: 132, 228, 284, 330, or 440;
SEQ ID NO: 416, 72, 129, or 204;
SEQ ID NO: 371, 425, 20, or 111; or
SEQ ID NO: 367, 407, 24, 93, or 218;
   wherein the modified oligonucleotide comprises at least one modification selected from a modified sugar and a modified internucleoside linkage.

Embodiment 9. The oligomeric compound of any of embodiments 1-8, wherein the nucleobase sequence of the modified oligonucleotide is at least 80%, 85%, 90%, 95%, or 100% complementary to any of the nucleobase sequences of SEQ ID NO: 1 or SEQ ID NO: 2 when measured across the entire nucleobase sequence of the modified oligonucleotide.

Embodiment 10. The oligomeric compound of any of embodiments 1-9, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 11. The oligomeric compound of any of embodiments 1-10, wherein the modified oligonucleotide comprises at least one modified nucleoside.

Embodiment 12. The oligomeric compound of embodiment 11, wherein the modified oligonucleotide comprises at least one modified nucleoside comprising a modified sugar moiety.

Embodiment 13. The oligomeric compound of embodiment 12, wherein the modified sugar moiety comprises a bicyclic sugar moiety.

Embodiment 14. The oligomeric compound of embodiment 13, wherein the bicyclic sugar moiety comprises a 2′-4′ bridge selected from —O—CH₂—; and —O—CH(CH₃)—.

Embodiment 15. The oligomeric compound of any of embodiments 11-14, wherein at least one modified nucleoside of the modified oligonucleotide comprises a non-bicyclic modified sugar moiety.

Embodiment 16. The oligomeric compound of embodiment 15, wherein at least one modified nucleoside of the modified oligonucleotide comprises a bicyclic sugar moiety having a 2′-4′ bridge and at least one nucleoside comprising a non-bicyclic modified sugar moiety.

Embodiment 17. The oligomeric compound of embodiment 15 or embodiment 16, wherein the non-bicyclic modified sugar moiety is a 2′-O(CH₂)₂—OCH₃ ribosyl sugar moiety, a 2′-OMe sugar moiety, or a 2′-F sugar moiety.

Embodiment 18. The oligomeric compound of any of embodiments 1-17, wherein the modified oligonucleotide comprises at least one modified nucleoside comprising a sugar surrogate.

Embodiment 19. The oligomeric compound of embodiment 18, wherein at least one modified nucleoside of the modified oligonucleotide comprises a sugar surrogate selected from morpholino and PNA.

Embodiment 20. The oligomeric compound of any of embodiments 1-19, wherein the modified oligonucleotide comprises at least one modified internucleoside linkage.

Embodiment 21. The oligomeric compound of embodiment 20, wherein each internucleoside linkage of the modified oligonucleotide is a modified internucleoside linkage.

Embodiment 22. The oligomeric compound of embodiment 20 or embodiment 21, wherein at least one internucleoside linkage is a phosphorothioate internucleoside linkage.

Embodiment 23. The oligomeric compound of embodiment 20 or 22, wherein the modified oligonucleotide comprises at least one phosphodiester internucleoside linkage.

Embodiment 24. The oligomeric compound of any of embodiments 20, 22, or 23, wherein each internucleoside linkage is independently selected from a phosphodiester internucleoside linkage or a phosphorothioate internucleoside linkage.

Embodiment 25. The oligomeric compound of any of embodiments 20 or 22-24, wherein at least 4, at least 5, at least 6, at least 7, at least 8, at least 9, at least 10, at least 11, at least 12, at least 13, at least 14, at least 15, at least 16, at least 17, at least 18, or at least 19 internucleoside linkages of the modified oligonucleotide are phosphorothioate internucleoside linkages.

Embodiment 26. The oligomeric compound of any of embodiments 20-22, 24, or 25, wherein each internucleoside linkage is a phosphorothioate internucleoside linkage.

Embodiment 27. The oligomeric compound of any of embodiments 20 or 22-26, wherein the internucleoside linkage motif of the modified oligonucleotide is selected from: 5′-sssssssssssssssssss-3′, 5′-sssssssssssssss-3′, 5′-soooossssssssssooss-3′, and ssooooooooooooooooooss; wherein each ‘o’ represents a phosphodiester internucleoside linkage and each ‘s’ represents a phosphorothioate internucleoside linkage.

Embodiment 28. The oligomeric compound of any of embodiments 1-27, wherein the modified oligonucleotide comprises a modified nucleobase.

Embodiment 29. The oligomeric compound of embodiment 28, wherein the modified nucleobase is a 5-methylcytosine.

Embodiment 30. The oligomeric compound of any of embodiments 1-29, wherein the oligomeric compound comprises a modified oligonucleotide consisting of 12-22, 12-20, 14-18, 14-20, 15-17, 15-25, 16-20, 16-18, 18-22, 18-25, 18-20, 20-25, or 21-23 linked nucleosides, or a pharmaceutically acceptable salt thereof.

Embodiment 31. The oligomeric compound of embodiment 30, wherein the modified oligonucleotide is a pharmaceutically acceptable salt comprising one or more cations selected from sodium, potassium, calcium, and magnesium.

Embodiment 32. The oligomeric compound of any of embodiments 1-31, wherein the modified oligonucleotide consists of 16 linked nucleosides.

Embodiment 33. The oligomeric compound of any of embodiments 1-31, wherein the modified oligonucleotide consists of 18 linked nucleosides.

Embodiment 34. The oligomeric compound of any of embodiments 1-31, wherein the modified oligonucleotide consists of 20 linked nucleosides.

Embodiment 35. The oligomeric compound of any of embodiments 1-31, wherein the modified oligonucleotide consists of 21 linked nucleosides.

Embodiment 36. The oligomeric compound of any of embodiments 1-31, wherein the modified oligonucleotide consists of 23 linked nucleosides.

Embodiment 37. The oligomeric compound of any of embodiments 1-35, wherein the oligomeric compound activates RNase H.

Embodiment 38. The oligomeric compound of embodiment 37, wherein the modified oligonucleotide is a gapmer.

Embodiment 39. The oligomeric compound of any of embodiments 1-34, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides;
wherein the 3′-most nucleoside of the 5′-region and the 5′-most nucleoside of the 3′-region comprise modified sugar moieties, and
each of the central region nucleosides is selected from a nucleoside comprising a 2′-β-D-deoxyribosyl sugar moiety and a nucleoside comprising a 2′-substituted sugar moiety, wherein the central region comprises at least six nucleosides comprising a 2′-β-D-deoxyribosyl sugar moiety and no more than two nucleosides comprising a 2′-substituted sugar moiety.

Embodiment 40. The oligomeric compound of any of embodiments 1-39, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

Embodiment 41. The oligomeric compound of embodiment 40, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises a 2′-O(CH₂)₂—OCH₃ ribosyl modified sugar moiety, and each of the central region nucleosides comprises a 2′-β-D-deoxyribosyl sugar moiety.

Embodiment 42. The oligomeric compound of embodiment 40, wherein the modified oligonucleotide has a sugar motif comprising:
a 5′-region consisting of 3 linked 5′-region nucleosides;
a central region consisting of 10 linked central region nucleosides; and
a 3′-region consisting of 3 linked 3′-region nucleosides; wherein
each of the 5′-region nucleosides and each of the 3′-region nucleosides comprises a cEt sugar moiety, and each of the central region nucleosides comprises a 2′-β-D-deoxyribosyl sugar moiety.

Embodiment 43. A chirally enriched population of oligomeric compounds of any of embodiments 1-42, wherein the population is enriched for modified oligonucleotides comprising at least one particular phosphorothioate internucleoside linkage having a particular stereochemical configuration.

Embodiment 44. The chirally enriched population of embodiment 43, wherein the population is enriched for modified oligonucleotides comprising at least one particular phosphorothioate internucleoside linkage having the (Sp) or (Rp) configuration.

Embodiment 45. The chirally enriched population of embodiment 43, wherein the population is enriched for modified oligonucleotides having a particular, independently selected stereochemical configuration at each phosphorothioate internucleoside linkage.

Embodiment 46. The chirally enriched population of embodiment 43, wherein the population is enriched for modified oligonucleotides having the (Rp) configuration at one particular phosphorothioate internucleoside linkage and the (Sp) configuration at each of the remaining phosphorothioate internucleoside linkages.

Embodiment 47. The chirally enriched population of embodiment 43, wherein the population is enriched for modified oligonucleotides having at least 3 contiguous phosphorothioate internucleoside linkages in the Sp, Sp, and Rp configurations, in the 5′ to 3′ direction.

Embodiment 48. A population of oligomeric compounds of any of embodiments 1-42, wherein all of the phosphorothioate internucleoside linkages of the modified oligonucleotide are stereorandom.

Embodiment 49. An oligomeric duplex, comprising a first oligomeric compound and a second oligomeric compound comprising a second modified oligonucleotide, wherein the first oligomeric compound is an oligomeric compound of any of embodiments 1-42.

Embodiment 50. The oligomeric duplex of embodiment 49, wherein the second oligomeric compound comprises a second modified oligonucleotide consisting of 12 to 50 linked nucleosides, and wherein the nucleobase sequence of the second modified oligonucleotide comprises a complementary region of at least 12 nucleobases that is at least 90% complementary to an equal length portion of the first modified oligonucleotide.

Embodiment 51. An oligomeric duplex comprising:
a first oligomeric compound comprising a first modified oligonucleotide consisting of 19 to 30 linked nucleosides wherein the nucleobase sequence of the first modified oligonucleotide comprises at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 561-1028; and
a second oligomeric compound comprising a second modified oligonucleotide consisting of 15 to 30 linked nucleosides wherein the nucleobase sequence of the second modified oligonucleotide comprises a complementary region of at least 12 nucleobases that is at least 90% complementary to an equal length portion of the first modified oligonucleotide.

Embodiment 52. An oligomeric duplex comprising:
a first oligomeric compound comprising a first modified oligonucleotide consisting of 19 to 30 linked nucleosides wherein the nucleobase sequence of the first modified oligonucleotide comprises at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 561-1028; and
   a second oligomeric compound comprising a second modified oligonucleotide consisting of 15 to 30 linked nucleosides wherein the nucleobase sequence of the second modified oligonucleotide comprises at least 12, at least 13, at least 14, at least 15, at least 16, at least 17, at least 18, at least 19, at least 20, or at least 21 contiguous nucleobases of the nucleobase sequence of any of SEQ ID NOs: 1029-1496, wherein the nucleobase sequence of the second modified oligonucleotide is at least 90% complementary to an equal length portion of the first modified oligonucleotide.

Embodiment 53. An oligomeric duplex comprising:
a first oligomeric compound comprising a first modified oligonucleotide consisting of 23 linked nucleosides wherein the nucleobase sequence of the first modified oligonucleotide comprises the nucleobase sequence of any of SEQ ID NOs: 561-1028; and
a second oligomeric compound comprising a second modified oligonucleotide consisting of 21 linked nucleosides wherein the nucleobase sequence of the second modified oligonucleotide comprises the nucleobase sequence of any of SEQ ID NOs: 1029-1496, wherein the nucleobase sequence of the second modified oligonucleotide is at least 90% complementary to an equal length portion of the first modified oligonucleotide.

Embodiment 54. The oligomeric duplex of any of embodiments 49-53, wherein the modified oligonucleotide of the first oligomeric compound comprises a 5′-stabilized phosphate group.

Embodiment 55. The oligomeric duplex of embodiment 54, wherein the 5′-stabilized phosphate group comprises a cyclopropyl phosphonate or a vinyl phosphonate.

Embodiment 56. The oligomeric duplex of any of embodiments 49-53, wherein the modified oligonucleotide of the first oligomeric compound comprises a glycol nucleic acid (GNA) sugar surrogate.

Embodiment 57. The oligomeric duplex of any of embodiments 49-55, wherein the modified oligonucleotide of the first oligomeric compound comprises a 2′-NMA sugar moiety.

Embodiment 58. The oligomeric duplex of any of embodiments 49-57, wherein at least one nucleoside of the second modified oligonucleotide comprises a modified sugar moiety.

Embodiment 59. The oligomeric duplex of embodiment 58, wherein the modified sugar moiety of the second modified oligonucleotide comprises a bicyclic sugar moiety.

Embodiment 60. The oligomeric duplex of embodiment 59, wherein the bicyclic sugar moiety of the second modified oligonucleotide comprises a 2′-4′ bridge selected from —O—CH₂—; and —O—CH(CH₃)—.

Embodiment 61. The oligomeric duplex of embodiment 59 or embodiment 60, wherein the modified sugar moiety of the second modified oligonucleotide comprises a non-bicyclic modified sugar moiety.

Embodiment 62. The oligomeric duplex of embodiment 61, wherein the non-bicyclic modified sugar moiety of the second modified oligonucleotide is a 2′-MOE sugar moiety, a 2′-F sugar moiety, or 2′-OMe sugar moiety.

Embodiment 63. The oligomeric duplex of any of embodiments 49-62, wherein at least one nucleoside of the second modified oligonucleotide comprises a sugar surrogate.

Embodiment 64. The oligomeric duplex of any of embodiments 49-63, wherein the second modified oligonucleotide comprises at least one modified internucleoside linkage.

Embodiment 65. The oligomeric duplex of embodiment 64, wherein at least one modified internucleoside linkage of the second modified oligonucleotide is a phosphorothioate internucleoside linkage.

Embodiment 66. The oligomeric duplex of any of embodiments 49-65, wherein the second modified oligonucleotide comprises at least one phosphodiester internucleoside linkage.

Embodiment 67. The oligomeric duplex of any of embodiments 49-66, wherein each internucleoside linkage of the second modified oligonucleotide is independently selected from a phosphodiester or a phosphorothioate internucleoside linkage.

Embodiment 68. The oligomeric duplex of any of embodiments 49-67, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.

Embodiment 69. The oligomeric duplex of any of embodiments 49-68, wherein the second modified oligonucleotide comprises at least one modified nucleobase.

Embodiment 70. The oligomeric duplex of embodiment 69, wherein the modified nucleobase of the second modified oligonucleotide is 5-methylcytosine.

Embodiment 71. The oligomeric duplex of any of embodiments 49-70, wherein the second modified oligonucleotide comprises a conjugate group.

Embodiment 72. The oligomeric duplex of embodiment 71, wherein the conjugate group comprises a conjugate linker and a conjugate moiety.

Embodiment 73. The oligomeric duplex of embodiment 71 or embodiment 72, wherein the conjugate group is attached to the second modified oligonucleotide at the 5′-end of the second modified oligonucleotide.

Embodiment 74. The oligomeric duplex of embodiment 71 or embodiment 72, wherein the conjugate group is attached to the second modified oligonucleotide at the 3′-end of the modified oligonucleotide.

Embodiment 75. The oligomeric duplex of embodiment 71 or embodiment 72, wherein the conjugate group is attached to the second modified oligonucleotide through a modified internucleoside linkage.

Embodiment 76. The oligomeric duplex of any of embodiments 71-75, wherein the conjugate group comprises a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 77. The oligomeric duplex of any of embodiments 71-76, wherein the conjugate moiety is a 6-palmitamidohexyl conjugate moiety.

Embodiment 78. The oligomeric duplex of any of embodiments 71-74, wherein the conjugate group has the following structure:

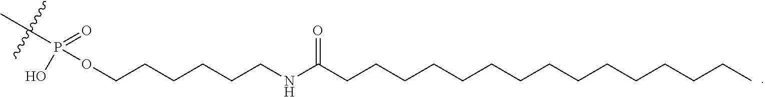 [SMILES: *P(=O)(O)OCCCCCCNC(=O)CCCCCCCCCCCCCCC]

Embodiment 79. The oligomeric duplex of any of embodiments 71-78, wherein the conjugate group comprises a cell-targeting moiety.

Embodiment 80. The oligomeric duplex of any of embodiments 49-79, wherein the second modified oligonucleotide comprises a terminal group.

Embodiment 81. The oligomeric duplex of embodiment 80, wherein the terminal group is an abasic sugar moiety.

Embodiment 82. The oligomeric duplex of any of embodiments 49-81, wherein the second modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 83. The oligomeric duplex of any of embodiments 49-81, wherein the modified oligonucleotide of the first oligomeric compound consists of 23 linked nucleosides and the second modified oligonucleotide consists of 21 linked nucleosides.

Embodiment 84. The oligomeric duplex of embodiment 83, wherein the modified oligonucleotide of the first oligomeric compound has a sugar motif (from 5′ to 3′) of: yfyyyfyyyyyyyfyfyyyyyyy and the second modified oligonucleotide has a sugar motif (from 5′ to 3′) of: yyyyyyfyfffyyyyyyyyyy, wherein each “y” represents a 2′-OMe sugar moiety and each “f” represents a 2′-F sugar moiety.

Embodiment 85. An antisense agent comprising an antisense compound, wherein the antisense compound is the oligomeric compound of any of embodiments 1-42.

Embodiment 86. An antisense agent, wherein the antisense agent is the oligomeric duplex of any of embodiments 49-84.

Embodiment 87. The antisense agent of embodiment 85 or embodiment 86, wherein the antisense agent is:
i. an RNase H agent capable of reducing the amount of PCDH19 nucleic acid through the activation of RNase H; or
ii. an RNAi agent capable of reducing the amount of PCDH19 nucleic acid through the activation of RISC/Ago2.

Embodiment 88. The antisense agent of any of embodiments 85-87, wherein the conjugate group is a cell-targeting moiety.

Embodiment 89. A pharmaceutical composition comprising an oligomeric compound of any of embodiments 1-42, a population of any of embodiments 43-48, an oligomeric duplex of any of embodiments 49-84, or an antisense agent of any of embodiments 85-88, and a pharmaceutically acceptable diluent.

Embodiment 90. The pharmaceutical composition of embodiment 89, wherein the pharmaceutically acceptable diluent is artificial cerebrospinal fluid (aCSF) or PBS.

Embodiment 91. The pharmaceutical composition of embodiment 90, wherein the pharmaceutical composition consists essentially of the oligomeric compound, the population, the oligomeric duplex, or the antisense agent, and aCSF.

Embodiment 92. The pharmaceutical composition of embodiment 90, wherein the pharmaceutical composition consists essentially of the oligomeric compound, the population, the oligomeric duplex, or the antisense agent, and PBS.

Embodiment 93. A method comprising administering to a subject an oligomeric compound of any of embodiments 1-42, a population of any of embodiments 43-48, an oligomeric duplex of any of embodiments 49-84, an antisense agent of any of embodiments 85-88, or a pharmaceutical composition of any of embodiments 89-92.

Embodiment 94. The method of embodiment 93, wherein the subject has a disease associated with PCDH19.

Embodiment 95. The method of embodiment 93, wherein the subject has PCDH19 Epilepsy.

Embodiment 96. A method of treating a disease associated with PCDH19 comprising administering to a subject having or at risk for developing a disease associated with PCDH19 a therapeutically effective amount of an oligomeric compound of any of embodiments 1-42, a population of any of embodiments 43-48, an oligomeric duplex of any of embodiments 49-84, an antisense agent of any of embodiments 85-88, or a pharmaceutical composition of any of embodiments 89-92; and thereby treating the disease associated with PCDH19.

Embodiment 97. The method of embodiment 96, wherein the disease associated with PCDH19 is a neurodevelopmental disease.

Embodiment 98. The method of embodiment 96 or embodiment 97, wherein the disease associated with PCDH19 is PCDH19 Epilepsy.

Embodiment 99. The method of any of embodiments 96-98, wherein at least one symptom or hallmark of the disease associated with PCDH19 is ameliorated.

Embodiment 100. The method of embodiment 99, wherein the symptom or hallmark is seizures, cognitive impairment, intellectual disabilities, autism spectrum disorder, behavioral problems, aggression, anxiety, obsessive-compulsive disorder, hyperactivity, attention deficit disorder (ADD), or attention deficit hyperactivity disorder (ADHD).

Embodiment 101. The method of embodiment 100, wherein the seizures are any of clusters of seizures, generalized tonic-clonic seiaures, focal seizures, or bilateral seizures.

Embodiment 102. The method of any of embodiments 96-101, wherein administering an oligomeric compound of any of embodiments 1-42, a population of any of embodiments 43-48, an oligomeric duplex of any of embodiments 49-84, an antisense agent of any of embodiments 85-88, or a pharmaceutical composition of any of embodiments 89-92 reduces seizures, reduces or delays cognitive impairment, reduces or delays intellectual disabilities, reduces or delays symptoms of autism spectrum disorder, reduces behavioral problems, reduces aggression, reduces anxiety, reduces obsessive-compulsive behavior, reduces hyperactivity, reduces symptoms of attention deficit disorder (ADD), or reduces symptoms of attention deficit hyperactivity disorder (ADHD) in the subject.

Embodiment 103. The method of any of embodiments 93-102, wherein the subject is human.

Embodiment 104. A method of reducing expression of PCDH19 in a cell comprising contacting the cell with an oligomeric compound of any of embodiments 1-42, a population of any of embodiments 43-48, an oligomeric duplex of any of embodiments 49-84, an antisense agent of any of embodiments 85-88, or a pharmaceutical composition of any of embodiments 89-92.

Embodiment 105. The method of embodiment 104, wherein the cell is a neuron.

Embodiment 106. The method of embodiment 104 or embodiment 105, wherein the cell is a human cell.

Embodiment 107. Use of an oligomeric compound of any of embodiments 1-42, a population of any of embodiments 43-48, an oligomeric duplex of any of embodiments 49-84, an antisense agent of any of embodiments 85-88, or a pharmaceutical composition of any of embodiments 89-92 for treating a disease associated with PCDH19.

Embodiment 108. Use of an oligomeric compound of any of embodiments 1-42, a population of any of embodiments 43-48, an oligomeric duplex of any of embodiments 49-84, or an antisense agent of any of embodiments 85-88 in the manufacture of a medicament for treating a disease associated with PCDH19.

Embodiment 109. The use of embodiment 107 or embodiment 108, wherein the disease associated with PCDH19 is PCDH19 Epilepsy.

### Certain Oligomeric Agents and Oligomeric Compounds

Certain embodiments provide oligomeric agents targeted to a PCDH19 nucleic acid. In certain embodiments, the PCDH19 nucleic acid has the sequence set forth in SEQ ID NO: 1 (ENSEMBL Accession No. ENSG00000165194.15 from version 104: May 2021), to SEQ ID NO: 2 (the cDNA of ENSEMBL Accession No. ENST00000373034.8 from version 104: May2021), or to both., each of which is incorporated by reference in its entirety. In certain embodiments, the oligomeric agent is a single-stranded oligomeric compound. In certain embodiments, the oligomeric agent is oligomeric duplex.

Certain embodiments provide an oligomeric compound comprising a modified oligonucleotide consisting of 12 to 50 linked nucleosides, wherein the nucleobase sequence of the modified oligonucleotide is at least 80% complementary to an equal length portion of a PCDH19 nucleic acid, and wherein the modified oligonucleotide has at least one modification selected from a modified sugar moiety and a modified internucleoside linkage. In certain embodiments, the PCDH19 nucleic acid has the nucleobase sequence of SEQ ID NOs: 1 or 2.

Certain embodiments provide an oligomeric compound comprising a modified oligonucleotide consisting of 12 to 50 linked nucleosides, wherein the nucleobase sequence of the modified oligonucleotide comprises at least 12, at least 13, at least 14, at least 15, or at least 16 contiguous nucleobases of any of the nucleobase sequences of SEQ ID NOs: 15-560.

Certain embodiments provide an oligomeric compound comprising a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, or 20 contiguous nucleobases of any of the nucleobase sequences of SEQ ID NOs: 15-482

Certain embodiments provide an oligomeric compound comprising a modified oligonucleotide consisting of 16 to 50 linked nucleosides, wherein the nucleobase sequence of the modified oligonucleotide comprises the nucleobase sequence of any of nucleobase sequences of SEQ ID NOs: 15-560.

Certain embodiments provide an oligomeric compound comprising a modified oligonucleotide consisting of 20 linked nucleosides, wherein the modified oligonucleotide has a nucleobase sequence consisting of the nucleobase sequence of any of the nucleobase sequences of SEQ ID NOs: 15-482.

Certain embodiments provide an oligomeric compound comprising a modified oligonucleotide consisting of 16 linked nucleosides, wherein the modified oligonucleotide has a nucleobase sequence consisting of the nucleobase sequence of any of the nucleobase sequences of SEQ ID NOs: 483-560.

In any of the oligomeric compounds provided herein, the nucleobase sequence of the modified oligonucleotide can be at least 85%, at least 90%, at least 95%, or 100% complementary to an equal length portion of a PCDH19 nucleic acid, wherein the PCDH19 nucleic acid has the nucleobase sequence of SEQ ID NOs: 1 or 2.

In any of the oligomeric compounds provided herein, the modified oligonucleotide can consist of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In any of the oligomeric compounds provided herein, at least one nucleoside of the modified oligonucleotide can comprise a modified sugar moiety. In certain embodiments, the modified sugar moiety comprises a bicyclic sugar moiety, such as a 2′-4′ bridge selected from —O—CH₂—; and —O—CH(CH₃)—. In certain embodiments, the modified sugar moiety comprises a non-bicyclic sugar moiety, such as a 2′-MOE sugar moiety or 2′-OMe sugar moiety.

In any of the oligomeric compounds provided herein, at least one nucleoside of the modified oligonucleotide compound can comprise a sugar surrogate.

In any of the oligomeric compounds provided herein, at least one internucleoside linkage of the modified oligonucleotide can comprise a modified internucleoside linkage, such as a phosphorothioate internucleoside linkage. In certain embodiments, each internucleoside linkage of the modified oligonucleotide can be a modified internucleoside linkage or each internucleoside linkage of the modified oligonucleotide can be a phosphorothioate internucleoside linkage. In certain embodiments, at least one internucleoside linkage of the modified oligonucleotide can be a phosphodiester internucleoside linkage. In certain embodiments, each internucleoside linkage of the modified oligonucleotide can be independently selected from a phosphodiester or a phosphorothioate internucleoside linkage. In certain embodiments, at least 2, at least 3, at least 4, at least 5, or at least 6 internucleoside linkages of the modified oligonucleotide can be phosphodiester internucleoside linkages. In certain embodiments, at least 10, at least 11, at least 12, at least 13, at least 14, at least 15, at least 16, at least 17, or at least 18 internucleoside linkages of the modified oligonucleotide can be phosphorothioate internucleoside linkages.

In any of the oligomeric compounds provided herein, at least one nucleobase of the modified oligonucleotide can be a modified nucleobase, such as 5-methylcytosine. In certain embodiments, each cytosine is 5-methylcytosine.

In any of the oligomeric compounds provided herein, the modified oligonucleotide can comprise a deoxy region consisting of 5-12 contiguous 2′-deoxynucleosides. In certain embodiments, each nucleoside of the deoxy region is a 2′-β-D-deoxynucleoside. In certain embodiments, the deoxy region consists of 7, 8, 9, 10, or 7-10 linked nucleosides. In certain embodiments, each nucleoside immediately adjacent to the deoxy region comprises a modified sugar moiety. In certain embodiments, the deoxy region is flanked on the 5′-side by a 5′-region consisting of 1-6 linked 5′-region nucleosides and on the 3′-side by a 3′-region consisting of 1-6 linked 3′-region nucleosides; wherein the 3′-most nucleoside of the 5′-region comprises a modified sugar moiety; and the 5′-most nucleoside of the 3′-region comprises a modified sugar moiety. In certain embodiments, each nucleoside of the 3′-region comprises a modified sugar moiety. In certain embodiments, each nucleoside of the 5′-region comprises a modified sugar moiety.

In certain embodiments, a compound comprises or consists of a modified oligonucleotide consisting of 16 to 50 linked nucleobases and having a nucleobase sequence comprising the nucleobase sequence recited in any one of SEQ ID NOs: 483-560, wherein the modified oligonucleotide has:
a gap segment consisting of ten linked 2′-deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein each nucleoside of each wing segment comprises a cEt nucleoside; wherein each internucleoside linkage is a phosphorothioate linkage; and wherein each cytosine is a 5-methylcytosine. In certain embodiments, the modified oligonucleotide consists of 16 to 30 linked nucleosides. In certain embodiments, the modified oligonucleotide consists of 16 linked nucleosides.

In certain embodiments, a compound comprises or consists of a modified oligonucleotide consisting of 16 to 50 linked nucleobases and having a nucleobase sequence comprising the nucleobase sequence recited in any one of SEQ ID NOs: 15-482, wherein the modified oligonucleotide has:
a gap segment consisting of ten linked 2′-deoxynucleosides;
a 5′ wing segment consisting of five linked nucleosides; and
a 3′ wing segment consisting of five linked nucleosides;
wherein the gap segment is positioned between the 5′ wing segment and the 3′ wing segment; wherein each nucleoside of each wing segment comprises a 2′-MOE nucleoside, and wherein each cytosine is a 5-methylcytosine. In certain embodiments, the modified oligonucleotide consists of 16 to 30 linked nucleosides. In certain embodiments, the modified oligonucleotide consists of 20 linked nucleosides wherein the internucleoside linkage motif for the modified oligonucleotide is (from 5′ to 3′): soooossssssssssooss; wherein each “s” represents a phosphorothioate internucleoside linkage and each “o” represents a phosphodiester internucleoside linkage.

In certain embodiments, an oligomeric compound comprises a conjugate group. In certain embodiments, the conjugate group comprises a conjugate linker and a conjugate moiety. In certain embodiments, the conjugate linker consists of a single bond, the conjugate linker is cleavable, the conjugate linker comprises 1-3 linker-nucleosides, the conjugate linker does not comprise any linker nucleosides, the conjugate group is attached to the modified oligonucleotide at the 5′-end of the modified oligonucleotide, or the conjugate group is attached to the modified oligonucleotide at the 3′-end of the modified oligonucleotide.

In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, the conjugate group has the following structure:

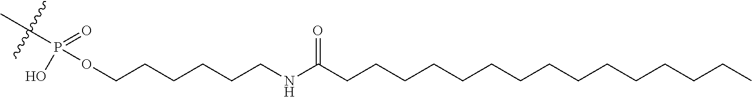 [SMILES: *P(=O)(O)OCCCCCCNC(=O)CCCCCCCCCCCCCCC]

### Certain Oligomeric Duplexes

Certain embodiments are directed to oligomeric duplexes comprising a first oligomeric compound and a second oligomeric compound.

In certain embodiments, an oligomeric duplex comprises:
a first oligomeric compound comprising a first modified oligonucleotide consisting of 12 to 50 linked nucleosides wherein the nucleobase sequence of the first modified oligonucleotide comprises at least at least 12, at least 13, at least 14, at least 15, at least 16, at least 17, at least 18, at least 19, or 20 contiguous nucleobases of the nucleobase sequence of any of SEQ ID NOs: 15-560; and
a second oligomeric compound comprising a second modified oligonucleotide consisting of 12 to 50 linked nucleosides wherein the nucleobase sequence of the second modified oligonucleotide comprises a complementary region of at least 8 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 16 to 50 linked nucleosides wherein the nucleobase sequence of the first modified oligonucleotide comprises the nucleobase sequence of any of SEQ ID NOs: 15-560; and
a second oligomeric compound comprising a second modified oligonucleotide consisting of 16 to 50 linked nucleosides wherein the nucleobase sequence of the second modified oligonucleotide comprises a complementary region of at least 16 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 19 to 30linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 561-1028, and
a second oligomeric compound comprising a second modified oligonucleotide consisting of 15 to 30 linked nucleosides wherein the nucleobase sequence of the second modified oligonucleotide comprises a complementary region of at least 8 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 19 to 30 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 561-1028, and
a second oligomeric compound comprising a second modified oligonucleotide consisting of 15 to 30 linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any of SEQ ID NOs: 1029-1496, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 23 linked nucleosides wherein the nucleobase sequence of the first modified oligonucleotide comprises the nucleobase sequence of any of SEQ ID NOs: 561-1028; and
a second oligomeric compound comprising a second modified oligonucleotide consisting of 21 linked nucleosides wherein the nucleobase sequence of the second modified oligonucleotide comprises the nucleobase sequence of any of SEQ ID NOs: 1029-1496, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises a first oligomeric compound comprising a first modified oligonucleotide consisting of 15 to 30 linked nucleosides and a second oligomeric compound comprising a second modified oligonucleotide consisting of 15 to 29 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs of nucleobase sequences recited in SEQ ID NOs: 723/1029, 724/1030, 725/1031, 561/1032, 562/1033, 563/1034, 726/1035, 727/1036, 728/1037, 564/1038, 729/1039, 730/1040, 731/1041, 732/1042, 733/1043, 565/1044, 734/1045, 566/1046, 735/1047, 567/1048, 736/1049, 737/1050, 738/1051, 568/1052, 739/1053, 740/1054, 741/1055, 742/1056, 743/1057, 569/1058, 744/1059, 745/1060, 746/1061, 747/1062, 748/1063, 570/1064, 749/1065, 571/1066, 750/1067, 751/1068, 572/1069, 573/1070, 574/1071, 575/1072, 576/1073, 577/1074, 578/1075, 752/1076, 753/1077, 579/1078, 580/1079, 754/1080, 581/1081, 582/1082, 755/1083, 756/1084, 583/1085, 584/1086, 757/1087, 758/1088, 585/1089, 586/1090, 587/1091, 588/1092, 589/1093, 590/1094, 591/1095, 592/1096, 759/1097, 593/1098, 594/1099, 760/1100, 595/1101, 596/1102, 761/1103, 597/1104, 598/1105, 762/1106, 763/1107, 599/1108, 764/1109, 765/1110, 766/1111, 600/1112, 767/1113, 768/1114, 769/1115, 770/1116, 601/1117, 771/1118, 602/1119, 772/1120, 773/1121, 774/1122, 603/1123, 604/1124, 775/1125, 605/1126, 606/1127, 776/1128, 777/1129, 778/1130, 779/1131, 780/1132, 781/1133, 782/1134, 783/1135, 784/1136, 607/1137, 785/1138, 786/1139, 787/1140, 788/1141, 608/1142, 609/1143, 610/1144, 789/1145, 611/1146, 790/1147, 612/1148, 613/1149, 791/1150, 614/1151, 792/1152, 615/1153, 793/1154, 616/1155, 794/1156, 795/1157, 617/1158, 796/1159, 797/1160, 798/1161, 618/1162, 799/1163, 619/1164, 800/1165, 801/1166, 620/1167, 621/1168, 802/1169, 803/1170, 804/1171, 805/1172, 622/1173, 806/1174, 807/1175, 808/1176, 809/1177, 623/1178, 810/1179, 811/1180, 624/1181, 812/1182, 625/1183, 813/1184, 626/1185, 814/1186, 815/1187, 627/1188, 816/1189, 817/1190, 818/1191, 628/1192, 819/1193, 629/1194, 820/1195, 630/1196, 821/1197, 631/1198, 632/1199, 822/1200, 823/1201, 824/1202, 825/1203, 633/1204, 634/1205, 826/1206, 827/1207, 635/1208, 636/1209, 828/1210, 637/1211, 638/1212, 829/1213, 830/1214, 831/1215, 832/1216, 833/1217, 834/1218, 639/1219, 640/1220, 835/1221, 641/1222, 836/1223, 642/1224, 837/1225, 838/1226, 839/1227, 840/1228, 841/1229, 842/1230, 643/1231, 644/1232, 843/1233, 645/1234, 646/1235, 844/1236, 845/1237, 846/1238, 647/1239, 847/1240, 848/1241, 849/1242, 850/1243, 851/1244, 852/1245, 648/1246, 853/1247, 854/1248, 855/1249, 856/1250, 857/1251, 858/1252, 859/1253, 649/1254, 650/1255, 651/1256, 860/1257, 861/1258, 862/1259, 863/1260, 864/1261, 652/1262, 653/1263, 865/1264, 866/1265, 867/1266, 868/1267, 654/1268, 869/1269, 655/1270, 870/1271, 871/1272, 656/1273, 872/1274, 657/1275, 873/1276, 658/1277, 659/1278, 874/1279, 875/1280, 660/1281, 876/1282, 877/1283, 878/1284, 879/1285, 880/1286, 661/1287, 662/1288, 663/1289, 881/1290, 882/1291, 883/1292, 884/1293, 885/1294, 664/1295, 886/1296, 887/1297, 888/1298, 665/1299, 666/1300, 667/1301, 889/1302, 668/1303, 890/1304, 891/1305, 892/1306, 669/1307, 893/1308, 670/1309, 671/1310, 894/1311, 672/1312, 673/1313, 895/1314, 896/1315, 674/1316, 897/1317, 675/1318, 676/1319, 677/1320, 898/1321, 899/1322, 900/1323, 901/1324, 678/1325, 902/1326, 679/1327, 903/1328, 904/1329, 905/1330, 906/1331, 907/1332, 908/1333, 680/1334, 909/1335, 910/1336, 911/1337, 912/1338, 913/1339, 681/1340, 914/1341, 915/1342, 916/1343, 917/1344, 918/1345, 919/1346, 920/1347, 921/1348, 922/1349, 923/1350, 924/1351, 925/1352, 926/1353, 927/1354, 682/1355, 928/1356, 929/1357, 930/1358, 683/1359, 684/1360, 931/1361, 932/1362, 933/1363, 934/1364, 935/1365, 685/1366, 936/1367, 937/1368, 938/1369, 939/1370, 940/1371, 941/1372, 942/1373, 943/1374, 944/1375, 945/1376, 946/1377, 947/1378, 948/1379, 949/1380, 950/1381, 951/1382, 686/1383, 687/1384, 688/1385, 689/1386, 952/1387, 690/1388, 953/1389, 691/1390, 954/1391, 692/1392, 693/1393, 694/1394, 695/1395, 955/1396, 956/1397, 957/1398, 958/1399, 696/1400, 959/1401, 697/1402, 960/1403, 698/1404, 699/1405, 961/1406, 962/1407, 963/1408, 964/1409, 965/1410, 966/1411, 967/1412, 700/1413, 701/1414, 968/1415, 702/1416, 969/1417, 970/1418, 971/1419, 972/1420, 973/1421, 703/1422, 704/1423, 974/1424, 975/1425, 705/1426, 976/1427, 977/1428, 978/1429, 979/1430, 980/1431, 981/1432, 982/1433, 983/1434, 984/1435, 985/1436, 706/1437, 986/1438, 987/1439, 707/1440, 988/1441, 989/1442, 708/1443, 709/1444, 990/1445, 991/1446, 992/1447, 993/1448, 710/1449, 994/1450, 995/1451, 996/1452, 711/1453, 997/1454, 998/1455, 999/1456, 1000/1457, 712/1458, 1001/1459, 1002/1460, 713/1461, 1003/1462, 1004/1463, 1005/1464, 1006/1465, 1007/1466, 1008/1467, 1009/1468, 714/1469, 715/1470, 716/1471, 1010/1472, 717/1473, 718/1474, 1011/1475, 1012/1476, 719/1477, 720/1478, 1013/1479, 1014/1480, 1015/1481, 1016/1482, 1017/1483, 1018/1484, 1019/1485, 1020/1486, 1021/1487, 1022/1488, 721/1489, 1023/1490, 722/1491, 1024/1492, 1025/1493, 1026/1494, 1027/1495, or 1028/1496, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, an oligomeric duplex comprises a first oligomeric compound comprising a first modified oligonucleotide consisting of 15 to 30 linked nucleosides and a second oligomeric compound comprising a second modified oligonucleotide consisting of 15 to 29 linked nucleosides, wherein the nucleobase sequence of the first modified oligonucleotide and the nucleobase sequence of the second modified oligonucleotide comprise any of the following pairs of nucleobase sequences recited in SEQ ID NOs: 723/1029, 724/1030, 725/1031, 561/1032, 562/1033, 563/1034, 726/1035, 727/1036, 728/1037, 564/1038, 729/1039, 730/1040, 731/1041, 732/1042, 733/1043, 565/1044, 734/1045, 566/1046, 735/1047, 567/1048, 736/1049, 737/1050, 738/1051, 568/1052, 739/1053, 740/1054, 741/1055, 742/1056, 743/1057, 569/1058, 744/1059, 745/1060, 746/1061, 747/1062, 748/1063, 570/1064, 749/1065, 571/1066, 750/1067, 751/1068, 572/1069, 573/1070, 574/1071, 575/1072, 576/1073, 577/1074, 578/1075, 752/1076, 753/1077, 579/1078, 580/1079, 754/1080, 581/1081, 582/1082, 755/1083, 756/1084, 583/1085, 584/1086, 757/1087, 758/1088, 585/1089, 586/1090, 587/1091, 588/1092, 589/1093, 590/1094, 591/1095, 592/1096, 759/1097, 593/1098, 594/1099, 760/1100, 595/1101, 596/1102, 761/1103, 597/1104, 598/1105, 762/1106, 763/1107, 599/1108, 764/1109, 765/1110, 766/1111, 600/1112, 767/1113, 768/1114, 769/1115, 770/1116, 601/1117, 771/1118, 602/1119, 772/1120, 773/1121, 774/1122, 603/1123, 604/1124, 775/1125, 605/1126, 606/1127, 776/1128, 777/1129, 778/1130, 779/1131, 780/1132, 781/1133, 782/1134, 783/1135, 784/1136, 607/1137, 785/1138, 786/1139, 787/1140, 788/1141, 608/1142, 609/1143, 610/1144, 789/1145, 611/1146, 790/1147, 612/1148, 613/1149, 791/1150, 614/1151, 792/1152, 615/1153, 793/1154, 616/1155, 794/1156, 795/1157, 617/1158, 796/1159, 797/1160, 798/1161, 618/1162, 799/1163, 619/1164, 800/1165, 801/1166, 620/1167, 621/1168, 802/1169, 803/1170, 804/1171, 805/1172, 622/1173, 806/1174, 807/1175, 808/1176, 809/1177, 623/1178, 810/1179, 811/1180, 624/1181, 812/1182, 625/1183, 813/1184, 626/1185, 814/1186, 815/1187, 627/1188, 816/1189, 817/1190, 818/1191, 628/1192, 819/1193, 629/1194, 820/1195, 630/1196, 821/1197, 631/1198, 632/1199, 822/1200, 823/1201, 824/1202, 825/1203, 633/1204, 634/1205, 826/1206, 827/1207, 635/1208, 636/1209, 828/1210, 637/1211, 638/1212, 829/1213, 830/1214, 831/1215, 832/1216, 833/1217, 834/1218, 639/1219, 640/1220, 835/1221, 641/1222, 836/1223, 642/1224, 837/1225, 838/1226, 839/1227, 840/1228, 841/1229, 842/1230, 643/1231, 644/1232, 843/1233, 645/1234, 646/1235, 844/1236, 845/1237, 846/1238, 647/1239, 847/1240, 848/1241, 849/1242, 850/1243, 851/1244, 852/1245, 648/1246, 853/1247, 854/1248, 855/1249, 856/1250, 857/1251, 858/1252, 859/1253, 649/1254, 650/1255, 651/1256, 860/1257, 861/1258, 862/1259, 863/1260, 864/1261, 652/1262, 653/1263, 865/1264, 866/1265, 867/1266, 868/1267, 654/1268, 869/1269, 655/1270, 870/1271, 871/1272, 656/1273, 872/1274, 657/1275, 873/1276, 658/1277, 659/1278, 874/1279, 875/1280, 660/1281, 876/1282, 877/1283, 878/1284, 879/1285, 880/1286, 661/1287, 662/1288, 663/1289, 881/1290, 882/1291, 883/1292, 884/1293, 885/1294, 664/1295, 886/1296, 887/1297, 888/1298, 665/1299, 666/1300, 667/1301, 889/1302, 668/1303, 890/1304, 891/1305, 892/1306, 669/1307, 893/1308, 670/1309, 671/1310, 894/1311, 672/1312, 673/1313, 895/1314, 896/1315, 674/1316, 897/1317, 675/1318, 676/1319, 677/1320, 898/1321, 899/1322, 900/1323, 901/1324, 678/1325, 902/1326, 679/1327, 903/1328, 904/1329, 905/1330, 906/1331, 907/1332, 908/1333, 680/1334, 909/1335, 910/1336, 911/1337, 912/1338, 913/1339, 681/1340, 914/1341, 915/1342, 916/1343, 917/1344, 918/1345, 919/1346, 920/1347, 921/1348, 922/1349, 923/1350, 924/1351, 925/1352, 926/1353, 927/1354, 682/1355, 928/1356, 929/1357, 930/1358, 683/1359, 684/1360, 931/1361, 932/1362, 933/1363, 934/1364, 935/1365, 685/1366, 936/1367, 937/1368, 938/1369, 939/1370, 940/1371, 941/1372, 942/1373, 943/1374, 944/1375, 945/1376, 946/1377, 947/1378, 948/1379, 949/1380, 950/1381, 951/1382, 686/1383, 687/1384, 688/1385, 689/1386, 952/1387, 690/1388, 953/1389, 691/1390, 954/1391, 692/1392, 693/1393, 694/1394, 695/1395, 955/1396, 956/1397, 957/1398, 958/1399, 696/1400, 959/1401, 697/1402, 960/1403, 698/1404, 699/1405, 961/1406, 962/1407, 963/1408, 964/1409, 965/1410, 966/1411, 967/1412, 700/1413, 701/1414, 968/1415, 702/1416, 969/1417, 970/1418, 971/1419, 972/1420, 973/1421, 703/1422, 704/1423, 974/1424, 975/1425, 705/1426, 976/1427, 977/1428, 978/1429, 979/1430, 980/1431, 981/1432, 982/1433, 983/1434, 984/1435, 985/1436, 706/1437, 986/1438, 987/1439, 707/1440, 988/1441, 989/1442, 708/1443, 709/1444, 990/1445, 991/1446, 992/1447, 993/1448, 710/1449, 994/1450, 995/1451, 996/1452, 711/1453, 997/1454, 998/1455, 999/1456, 1000/1457, 712/1458, 1001/1459, 1002/1460, 713/1461, 1003/1462, 1004/1463, 1005/1464, 1006/1465, 1007/1466, 1008/1467, 1009/1468, 714/1469, 715/1470, 716/1471, 1010/1472, 717/1473, 718/1474, 1011/1475, 1012/1476, 719/1477, 720/1478, 1013/1479, 1014/1480, 1015/1481, 1016/1482, 1017/1483, 1018/1484, 1019/1485, 1020/1486, 1021/1487, 1022/1488, 721/1489, 1023/1490, 722/1491, 1024/1492, 1025/1493, 1026/1494, 1027/1495, or 1028/1496, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein the nucleobase sequence of the first modified oligonucleotide and the nucleobase sequence of the second modified oligonucleotide consist of any of the following pairs of nucleobase sequences recited in SEQ ID NOs: 723/1029, 724/1030, 725/1031, 561/1032, 562/1033, 563/1034, 726/1035, 727/1036, 728/1037, 564/1038, 729/1039, 730/1040, 731/1041, 732/1042, 733/1043, 565/1044, 734/1045, 566/1046, 735/1047, 567/1048, 736/1049, 737/1050, 738/1051, 568/1052, 739/1053, 740/1054, 741/1055, 742/1056, 743/1057, 569/1058, 744/1059, 745/1060, 746/1061, 747/1062, 748/1063, 570/1064, 749/1065, 571/1066, 750/1067, 751/1068, 572/1069, 573/1070, 574/1071, 575/1072, 576/1073, 577/1074, 578/1075, 752/1076, 753/1077, 579/1078, 580/1079, 754/1080, 581/1081, 582/1082, 755/1083, 756/1084, 583/1085, 584/1086, 757/1087, 758/1088, 585/1089, 586/1090, 587/1091, 588/1092, 589/1093, 590/1094, 591/1095, 592/1096, 759/1097, 593/1098, 594/1099, 760/1100, 595/1101, 596/1102, 761/1103, 597/1104, 598/1105, 762/1106, 763/1107, 599/1108, 764/1109, 765/1110, 766/1111, 600/1112, 767/1113, 768/1114, 769/1115, 770/1116, 601/1117, 771/1118, 602/1119, 772/1120, 773/1121, 774/1122, 603/1123, 604/1124, 775/1125, 605/1126, 606/1127, 776/1128, 777/1129, 778/1130, 779/1131, 780/1132, 781/1133, 782/1134, 783/1135, 784/1136, 607/1137, 785/1138, 786/1139, 787/1140, 788/1141, 608/1142, 609/1143, 610/1144, 789/1145, 611/1146, 790/1147, 612/1148, 613/1149, 791/1150, 614/1151, 792/1152, 615/1153, 793/1154, 616/1155, 794/1156, 795/1157, 617/1158, 796/1159, 797/1160, 798/1161, 618/1162, 799/1163, 619/1164, 800/1165, 801/1166, 620/1167, 621/1168, 802/1169, 803/1170, 804/1171, 805/1172, 622/1173, 806/1174, 807/1175, 808/1176, 809/1177, 623/1178, 810/1179, 811/1180, 624/1181, 812/1182, 625/1183, 813/1184, 626/1185, 814/1186, 815/1187, 627/1188, 816/1189, 817/1190, 818/1191, 628/1192, 819/1193, 629/1194, 820/1195, 630/1196, 821/1197, 631/1198, 632/1199, 822/1200, 823/1201, 824/1202, 825/1203, 633/1204, 634/1205, 826/1206, 827/1207, 635/1208, 636/1209, 828/1210, 637/1211, 638/1212, 829/1213, 830/1214, 831/1215, 832/1216, 833/1217, 834/1218, 639/1219, 640/1220, 835/1221, 641/1222, 836/1223, 642/1224, 837/1225, 838/1226, 839/1227, 840/1228, 841/1229, 842/1230, 643/1231, 644/1232, 843/1233, 645/1234, 646/1235, 844/1236, 845/1237, 846/1238, 647/1239, 847/1240, 848/1241, 849/1242, 850/1243, 851/1244, 852/1245, 648/1246, 853/1247, 854/1248, 855/1249, 856/1250, 857/1251, 858/1252, 859/1253, 649/1254, 650/1255, 651/1256, 860/1257, 861/1258, 862/1259, 863/1260, 864/1261, 652/1262, 653/1263, 865/1264, 866/1265, 867/1266, 868/1267, 654/1268, 869/1269, 655/1270, 870/1271, 871/1272, 656/1273, 872/1274, 657/1275, 873/1276, 658/1277, 659/1278, 874/1279, 875/1280, 660/1281, 876/1282, 877/1283, 878/1284, 879/1285, 880/1286, 661/1287, 662/1288, 663/1289, 881/1290, 882/1291, 883/1292, 884/1293, 885/1294, 664/1295, 886/1296, 887/1297, 888/1298, 665/1299, 666/1300, 667/1301, 889/1302, 668/1303, 890/1304, 891/1305, 892/1306, 669/1307, 893/1308, 670/1309, 671/1310, 894/1311, 672/1312, 673/1313, 895/1314, 896/1315, 674/1316, 897/1317, 675/1318, 676/1319, 677/1320, 898/1321, 899/1322, 900/1323, 901/1324, 678/1325, 902/1326, 679/1327, 903/1328, 904/1329, 905/1330, 906/1331, 907/1332, 908/1333, 680/1334, 909/1335, 910/1336, 911/1337, 912/1338, 913/1339, 681/1340, 914/1341, 915/1342, 916/1343, 917/1344, 918/1345, 919/1346, 920/1347, 921/1348, 922/1349, 923/1350, 924/1351, 925/1352, 926/1353, 927/1354, 682/1355, 928/1356, 929/1357, 930/1358, 683/1359, 684/1360, 931/1361, 932/1362, 933/1363, 934/1364, 935/1365, 685/1366, 936/1367, 937/1368, 938/1369, 939/1370, 940/1371, 941/1372, 942/1373, 943/1374, 944/1375, 945/1376, 946/1377, 947/1378, 948/1379, 949/1380, 950/1381, 951/1382, 686/1383, 687/1384, 688/1385, 689/1386, 952/1387, 690/1388, 953/1389, 691/1390, 954/1391, 692/1392, 693/1393, 694/1394, 695/1395, 955/1396, 956/1397, 957/1398, 958/1399, 696/1400, 959/1401, 697/1402, 960/1403, 698/1404, 699/1405, 961/1406, 962/1407, 963/1408, 964/1409, 965/1410, 966/1411, 967/1412, 700/1413, 701/1414, 968/1415, 702/1416, 969/1417, 970/1418, 971/1419, 972/1420, 973/1421, 703/1422, 704/1423, 974/1424, 975/1425, 705/1426, 976/1427, 977/1428, 978/1429, 979/1430, 980/1431, 981/1432, 982/1433, 983/1434, 984/1435, 985/1436, 706/1437, 986/1438, 987/1439, 707/1440, 988/1441, 989/1442, 708/1443, 709/1444, 990/1445, 991/1446, 992/1447, 993/1448, 710/1449, 994/1450, 995/1451, 996/1452, 711/1453, 997/1454, 998/1455, 999/1456, 1000/1457, 712/1458, 1001/1459, 1002/1460, 713/1461, 1003/1462, 1004/1463, 1005/1464, 1006/1465, 1007/1466, 1008/1467, 1009/1468, 714/1469, 715/1470, 716/1471, 1010/1472, 717/1473, 718/1474, 1011/1475, 1012/1476, 719/1477, 720/1478, 1013/1479, 1014/1480, 1015/1481, 1016/1482, 1017/1483, 1018/1484, 1019/1485, 1020/1486, 1021/1487, 1022/1488, 721/1489, 1023/1490, 722/1491, 1024/1492, 1025/1493, 1026/1494, 1027/1495, or 1028/1496, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, the internucleoside linkage motif of the second modified oligonucleotide can be ssooooooooooooooooss, wherein wherein each “o” represents a phosphodiester internucleoside linkage and each “s” represents a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl,

C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of PCDH19 nucleic acid through the activation of RISC/Ago2. In certain embodiments, an antisense agent, which can comprise an oligomeric compound or an oligomeric duplex described herein, is an RNAse H agent capable of reducing the amount of PCDH19 nucleic acid through the activation of RNAse H. Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modified sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 3′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′—OCH₃ (“OMe” or “O-methyl”), and 2′-O(CH₂)₂OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ) (Rₙ) or OCH₂C(═O)—N(Rₘ) (Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl). In certain embodiments, non-bicyclic modified sugar moieties comprise a substituent group at the 4′-position. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, ethyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂(“DMAOE”), O(CH₂)₂O(CH₂)₂N(CH₃)₂ (“DMAEOE”) and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. n certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃) (CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, C(Rₐ)═C(R_{b})—, C(Rₐ)═N—, C(═NRₐ)—, —C(═O)—, —C(═S)—, —O—, —Si(Rₐ)₂—, —S(═O)ₓ—, and N(Rₐ)—;
wherein:
x is 0, 1, or 2;
n is 1, 2, 3, or 4;
each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJIJ2, SJ1, N3, COOJI, acyl (C(═O)—H), substituted acyl, CN, sulfonyl (S(═O) 2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Additional bicyclic sugar moieties are known in the art, see, for example: Freier et al., Nucleic Acids Research, 1997, 25 (22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727. In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

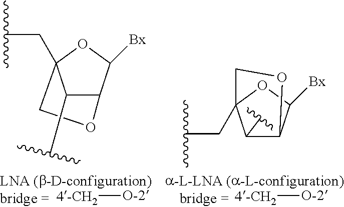

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33 (1): 439-447; Mook, OR. et al., (2007) Mal Cane Ther 6 (3): 833-843; Grunweller, A. et al., (2003) Nucleic Acids Research 31 (12): 3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

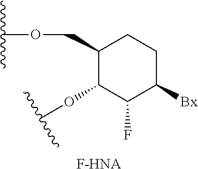

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

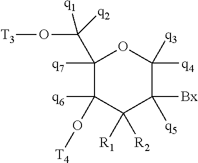

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X) NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, Q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

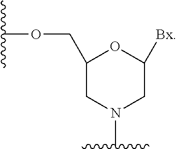

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

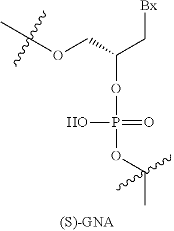

where Bx represents any nucleobase.

Many other bicyclic and tricyclic sugar and sugar surrogates are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimi-dines, alkyl or alkynyl substituted pyrimidines, alkyl substituted purines, and N-2, N-6 and O-6 substituted purines. In certain embodiments, modified nucleobases are selected from: 5-methylcytosine, 2-aminopropyladenine, 5-hydroxymethylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S.T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P—O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

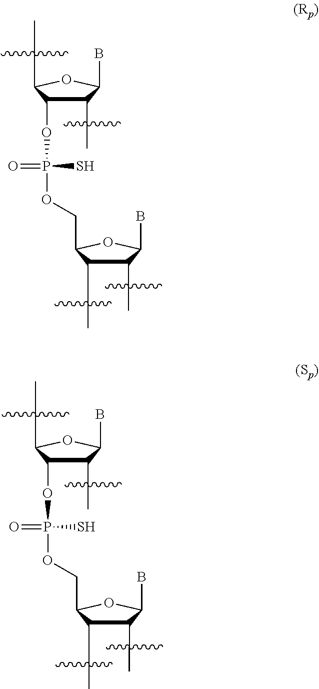

In certain embodiments, a modified internucleoside linkage comprises a linking group having a formula:

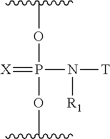

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O) R₃, and P(═O) R₄R₅, wherein:
   R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
   R₃ is selected from an aryl, a substituted aryl, CH₃, N(CH₃) 2, OCH₃ and a conjugate group;
   R₄ is selected from OCH₃, OH, C₁-C₆ alkyl, substituted C₁-C₆ alkyl and a conjugate group; and
   R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

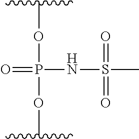 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

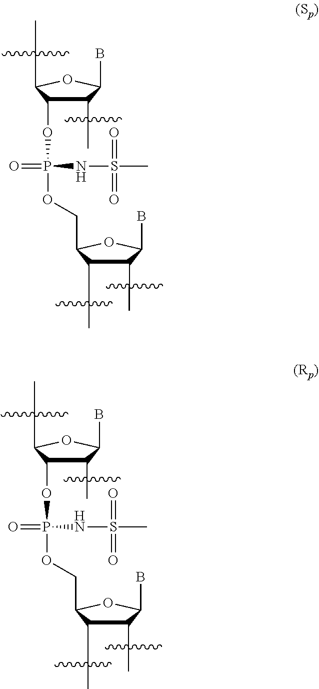

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)—5′), amide-4 (3′-CH₂—N(H)—C(═O)—5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

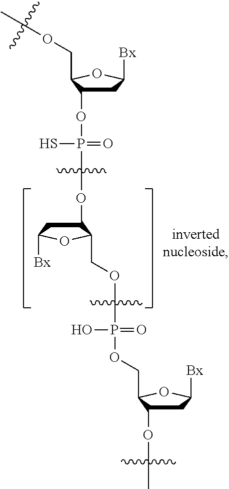

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

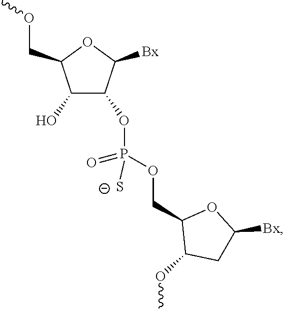

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein. Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, at least one nucleoside of the gap of a gapmer comprises a modified sugar moiety. In certain embodiments, one nucleoside of the gap comprises a modified sugar moiety and each remaining nucleoside of the gap comprises a 2′-deoxyribosyl sugar moiety. In certain embodiments, at least one nucleoside of the gap of a gapmer comprises a 2′-OMe sugar moiety.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification in each sugar moiety of each wing and the gap nucleosides comprise 2′-β-D-deoxyribosyl sugar moieties. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-10-5 MOE gapmer consists of 5 linked 2′-MOE nucleosides in the 5′-wing, 10 linked 2′-β-D-deoxynucleosides in the gap, and 5 linked 2′-MOE nucleosides in the 3′-wing.

In certain embodiments, modified oligonucleotides have the sugar motif from 5′ to 3′: eeeeeddddddddddeeeee; wherein each “d” represents a 2′-β-D-deoxyribosyl sugar moiety and each “e” represents a 2′-MOE ribosyl sugar moiety.

In certain embodiments, modified oligonucleotides have the sugar motif from 5′ to 3′: kkkddddddddddkkk; wherein each “d” represents a 2′-β-D-deoxyribosyl sugar moiety, and each “k” represents a cEt sugar moiety.

### 2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl sugar moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate.

In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### C. Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, conjugate groups may be selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, conjugate groups may be selected from any of C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, a conjugate group has the following structure:

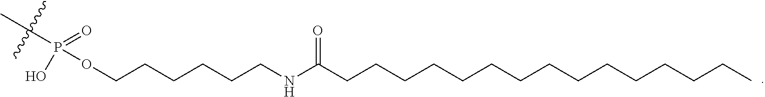 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)—(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

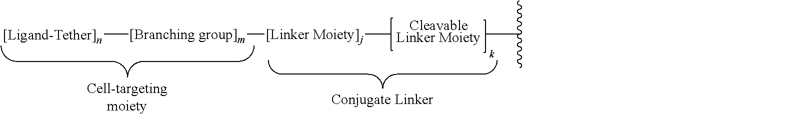

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, jis 1 and k is 1. In certain embodiments, n is 2, jis 1 and k is 0. In certain embodiments, n is 2, j is 0 and k is 1. In certain embodiments, n is 2, j is 1 and k is 1. In certain embodiments, n is 3, jis 1 and k is 0. In certain embodiments, n is 3, jis 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, oligomeric compounds comprise a conjugate group comprising a cell-targeting moiety having an affinity for transferrin receptor (TfR), also known as TfR1 and CD71. In certain embodiments, the conjugate group comprises an anti-TfR1 antibody or fragment thereof. In certain embodiments, the anti-TfR 1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′) 2, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic sugar moieties and/or inverted nucleosides. In certain embodiments, terminal groups comprise one or more 2′-linked nucleosides or sugar moieties. In certain such embodiments, the 2′-linked group is an abasic sugar moiety.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense agents. In certain embodiments, antisense agents have antisense activity when they reduce or inhibit the amount or activity of a target nucleic acid by 25% or more in the standard cell assay. In certain embodiments, antisense agents selectively affect one or more target nucleic acid. Such antisense agents comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense agents or a portion or an antisense agentto a target nucleic acid results in recruitment of a protein that cleaves the target nucleic acid. For example, certain antisense agents result in RNase H mediated cleavage of the target nucleic acid. RNase H is a cellular endonuclease that cleaves the RNA strand of an RNA: DNA duplex. The DNA in such an RNA: DNA duplex need not be unmodified DNA. In certain embodiments, described herein are antisense agents comprising antisense oligomeric compounds comprising antisense oligonucleotidesthat are sufficiently “DNA-like” to elicit RNase H activity. In certain embodiments, one or more non-DNA-like nucleoside in the gap of a gapmer is tolerated.

In certain antisense activities, an antisense agent or a portion of an antisense agent is loaded into an RNA-induced silencing complex (RISC), ultimately resulting in cleavage of the target nucleic acid. For example, certain antisense agents result in cleavage of the target nucleic acid by Argonaute. Antisense agents that are loaded into RISC are RNAi agents. RNAi agents may be double-stranded (siRNA or dsRNAi) or single-stranded (ssRNAi).

In certain embodiments, hybridization of an antisense agent or portion thereof to a target nucleic acid does not result in recruitment of a protein that cleaves that target nucleic acid. In certain embodiments, hybridization of the antisense agent or portion thereof to the target nucleic acid results in alteration of splicing of the target nucleic acid. In certain embodiments, hybridization of an antisense agent or a portion thereof to a target nucleic acid results in inhibition of a binding interaction between the target nucleic acid and a protein or other nucleic acid. In certain embodiments, hybridization of an antisense agent or a portion thereof to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or animal.

### IV. Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### A. Complementarity/Mismatches to the Target Nucleic Acid and Duplex Complementarity

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### B. PCDH19

In certain embodiments, oligomeric compounds comprise or consist of an oligonucleotide that is complementary to a target nucleic acid, wherein the target nucleic acid is a PCDH19 nucleic acid. In certain embodiments, the PCDH19 nucleic acid has the nucleobase sequence set forth in SEQ ID NO: 1 (ENSEMBL Accession No. ENSG00000165194.15 from version 104: May 2021), to SEQ ID NO: 2 (the cDNA of ENSEMBL Accession No. ENST00000373034.8 from version 104: May2021), or to both. In certain embodiments, contacting a cell with an oligomeric compound complementary to SEQ ID NO: 1 or SEQ ID NO: 2 reduces the amount of PCDH19 RNA, and in certain embodiments reduces the amount of PCDH19 protein. In certain embodiments, contacting a cell with an oligomeric compound complementary to SEQ ID NO: 1 or SEQ ID NO: 2 reduces the amount of PCDH19 RNA in a cell, and in certain embodiments reduces the amount of PCDH19 protein in a cell. In certain embodiments, the cell is in vitro. In certain embodiments, the oligomeric compound consists of a modified oligonucleotide. In certain embodiments, the oligomeric compound consists of a modified oligonucleotide and a conjugate group. In certain embodiments, the oligomeric compound is paired with an additional oligomeric compound in an oligomeric duplex. In certain embodiments, the oligomeric duplex comprises a conjugate group.

In certain embodiments, an oligomeric compound complementary to SEQ ID NO: 1 or SEQ ID NO: 2 is capable of reducing the detectable amount of PCDH19 RNA in vitro by at least 10%, at least 20%, at least 30%, at least 40%, at least 50%, at least 60%, at least 70%, at least 80%, or at least 90% in the standard cell assay. In certain embodiments, an oligomeric compound complementary to SEQ ID NO: 1 or SEQ ID NO: 2 is capable of reducing the detectable amount of PCDH19 protein in vitro by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2 is capable of reducing the detectable amount of PCDH19 RNA in vivo by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2 is capable of reducing the detectable amount of PCDH19 protein in vivo by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2, is capable of reducing the detectable amount of PCDH19 RNA in the CSF of an animal by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2, is capable of reducing the detectable amount of PCDH19 protein in the CSF of an animal by at least 10%, at least 20%, at least 30%, at least 40%, at least 50%, at least 60%, at least 70%, at least 80%, or at least 90%.

In certain embodiments, contacting a cell in an animal with the oligomeric compound ameliorates one or more symptom or hallmark of a neurodevelopmental disease or disorder. In certain embodiments, the neurodevelopmental disease or disorder is PCDH19 Epilepsy. In certain embodiments, the symptom or hallmark is any of seizures, cognitive impairment, intellectual disabilities, autism spectrum disorder, behavioral problems, aggression, anxiety, obsessive-compulsive disorder, hyperactivity, attention deficit disorder (ADD), and attention deficit hyperactivity disorder (ADHD). In certain embodiments, the seizures are any of clusters of seizures, generalized tonic-clonic seizures, or focal seizures, which may evolve to bilateral, tonic-clonic seizures.

### C. Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system. Such tissues include the brain and spinal cord.

### IV. Certain Methods and Uses

Certain embodiments provided herein relate to methods of reducing or inhibiting PCDH19 expression or activity, which can be useful for treating, preventing, or ameliorating a disease associated with PCDH19. In certain embodiments, the disease associated with PCDH19 is a neurodevelopmental disease. In certain embodiments, the disease associated with PCDH19 is PCDH19 Epilepsy.

In certain embodiments, a method comprises administering to a subject an oligomeric compound, a modified oligonucleotide, an oligomeric duplex, or an antisense agent, any of which having a nucleobase sequence complementary to a PCDH19 nucleic acid. In certain embodiments, the subject has a neurodevelopmental disease. In certain embodiments, the subject has PCDH19 Epilepsy.

In certain embodiments, a method of treating a disease associated with PCDH19 comprises administering to a subject an oligomeric compound, a modified oligonucleotide, an oligomeric duplex, or an antisense agent, any of which having a nucleobase sequence complementary to a PCDH19 nucleic acid. In certain embodiments, the subject has or is a risk of developing a disease associated with PCDH19. In certain embodiments, the subject has a neurodevelopmental disease. In certain embodiments, the subject has PCDH19 Epilepsy. In certain embodiments, at least one symptom or hallmark of the disease associated with PCDH19 is ameliorated. In certain embodiments, the at least one symptom or hallmark is seizures, cognitive impairment, intellectual disabilities, autism spectrum disorder, behavioral problems, aggression, anxiety, obsessive-compulsive disorder, hyperactivity, attention deficit disorder (ADD), or attention deficit hyperactivity disorder (ADHD). In certain embodiments, the seizures are any of clusters of seizures, generalized tonic-clonic seiaures, focal seizures, or bilateral seizures. In certain embodiments, administration of the oligomeric compound, the modified oligonucleotide, the oligomeric duplex, or the antisense agent to the subject reduces or delays the onset or progression of seizures, cognitive impairment, intellectual disabilities, autism spectrum disorder, behavioral problems, aggression, anxiety, obsessive-compulsive disorder, hyperactivity, attention deficit disorder (ADD), or attention deficit hyperactivity disorder (ADHD) in the subject.

In certain embodiments, a method of reducing expression of PCDH19 nucleic acid, for example RNA, or reducing expression of PCDH19 protein in a cell comprises contacting the cell with an oligomeric compound, a modified oligonucleotide, an oligomeric duplex, or an antisense agent, any of which having a nucleobase sequence complementary to a PCDH19 nucleic acid. In certain embodiments, the subject has or is a risk of developing a disease associated with PCDH19. In certain embodiments, the subject has a neurodevelopmental disease. In certain embodiments, the subject has PCDH19 Epilepsy. In certain embodiments, the cell is a neuron. In certain embodiments, the cell is a human cell.

Certain embodiments are drawn to an oligomeric compound, a modified oligonucleotide, an oligomeric duplex, or an antisense agent, any of which having a nucleobase sequence complementary to a PCDH19 nucleic acid, for use in treating a disease associated with PCDH19 or for use in the manufacture of a medicament for treating a disease associated with PCDH19. In certain embodiments, the disease associated with PCDH19 is a neurodevelopmental disease. In certain embodiments, the disease associated with PCDH19 is PCDH19 Epilepsy.

In any of the methods or uses described herein, the oligomeric compound, the modified oligonucleotide, the oligomeric duplex, or the antisense agent can be any described herein.

### V. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium and, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80TM and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80TM; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), intraneural, perineural, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salt thereof” or “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of a number of fully protonated molecules that weighs 10 mg. This would be equivalent to 10.59 mg of solvent-free, sodium acetate-free, anhydrous sodiated Compound No. 1549516. In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### VI. Certain Hotspot Regions

In certain embodiments, nucleobases in the ranges specified below comprise a hotspot region of a PCDH19 nucleic acid. In certain embodiments, modified oligonucleotides that are complementary to a hotspot region of PCDH19 nucleic acid achieve an average of more than 50% reduction of PCDH19 RNA in the standard in vitro assay. In certain embodiments, modified oligonucleotides that are complementary to a hotspot region of PCDH19 nucleic acid achieve an average of 50% or greater reduction of PCDH19 RNA in vivo in the standard in vivo assay.

### 1. Nucleobases 4743-4767 of SEQ ID NO: 1

In certain embodiments, nucleobases 4743-4767 of SEQ ID NO: 1 comprise a hotspot region. In certain embodiments, modified oligonucleotides are complementary to a portion of nucleobases 4743-4767 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the internucleoside linkages of the modified oligonucleotides are phosphorothioate internucleoside linkages and phosphodiester internucleoside linkages.

The nucleobase sequences of SEQ ID NOs: 132, 228, 284, 330, and 440 are complementary to nucleobases 4743-4767 of SEQ ID NO: 1.

The nucleobase sequences of Compound Nos: 1549744, 1549855, 1549749, 1549523, and 1549712 are complementary to nucleobases 4743-4767 of SEQ ID NO: 1.

In certain embodiments, modified oligonucleotides complementary to a portion of nucleobases 4743-4767 of SEQ ID NO: 1 achieve at least 81% reduction of PCDH19 mRNA in the standard in vitro assay. In certain embodiments, modified oligonucleotides complementary to a portion of nucleobases 4743-4767 of SEQ ID NO: 1 achieve an average of 86% reduction of PCDH19 mRNA in the standard in vitro assay.

### 2. Nucleobases 12,319-12,346 of SEQ ID NO: 1

In certain embodiments, nucleobases 12,319-12,346 of SEQ ID NO: 1 comprise a hotspot region. In certain embodiments, modified oligonucleotides are complementary to a portion of nucleobases 12,319-12,346 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the internucleoside linkages of the modified oligonucleotides are phosphorothioate internucleoside linkages and phosphodiester internucleoside linkages.

The nucleobase sequences of SEQ ID NOs: 416, 72, 129, and 204 are complementary to nucleobases 12,319-12,346 of SEQ ID NO: 1.

The nucleobase sequences of Compound Nos: 1549581, 1549876, 1549736, and 1549694 are complementary to nucleobases 12,319-12,346 of SEQ ID NO: 1.

In certain embodiments, modified oligonucleotides complementary to a portion of nucleobases 12,319-12,346 of SEQ ID NO: 1 achieve at least 65% reduction of PCDH19 mRNA in the standard in vitro assay. In certain embodiments, modified oligonucleotides complementary to a portion of nucleobases 12,319-12,346 of SEQ ID NO: 1 achieve an average of 69% reduction of PCDH19 mRNA in the standard in vitro assay.

### 3. Nucleobases 34364-34389 of SEQ ID NO: 1

In certain embodiments, nucleobases 34364-34389 of SEQ ID NO: 1 comprise a hotspot region. In certain embodiments, modified oligonucleotides are complementary to a portion of nucleobases 34364-34389 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the internucleoside linkages of the modified oligonucleotides are phosphorothioate internucleoside linkages and phosphodiester internucleoside linkages.

The nucleobase sequences of SEQ ID NOs: 371, 425, 20, and 111 are complementary to nucleobases 34364-34389 of SEQ ID NO: 1.

The nucleobase sequences of Compound Nos: 1549753, 1549642, 1549562, and 1549613 are complementary to nucleobases 34364-34389 of SEQ ID NO: 1.

In certain embodiments, modified oligonucleotides complementary to a portion of nucleobases 34364-34389 of SEQ ID NO: 1 achieve at least 64% reduction of 34389 of SEQ ID NO: 1 achieve an average of 80% reduction of PCDH19. In certain embodiments, modified oligonucleotides complementary to a portion of nucleobases 34364-34389 of SEQ ID NO: 1 achieve an average of 80% reduction of PCDH19 mRNA in the standard in vitro assay.

### 4. Nucleobases 84,408-84,431 of SEQ ID NO: 1

In certain embodiments, nucleobases 84,408-84,431 of SEQ ID NO: 1 comprise a hotspot region. In certain embodiments, modified oligonucleotides are complementary to a portion of nucleobases 84,408-84,431 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, the internucleoside linkages of the modified oligonucleotides are phosphorothioate internucleoside linkages and phosphodiester internucleoside linkages.

The nucleobase sequences of SEQ ID NOs: 367, 407, 24, 93, and 218 are complementary to nucleobases 84,408-84,431 of SEQ ID NO: 1.

The nucleobase sequences of Compound Nos: 1549714, 1549528, 1549617, 1549514, and 1549790 are complementary to nucleobases 84,408-84,431 of SEQ ID NO: 1.

In certain embodiments, modified oligonucleotides complementary to a portion of nucleobases 84,408-84,431 of SEQ ID NO: 1 achieve at least 65% reduction of PCDH19 mRNA in the standard in vitro assay. In certain embodiments, modified oligonucleotides complementary to a portion of nucleobases 84,408-84,431 of SEQ ID NO: 1 achieve an average of 74% reduction of PCDH19 mRNA in the standard in vitro assay.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT”CGAUCG,” wherein mC indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO: X, independent of any sugar or internucleoside linkage modifications also described in such SEQ ID.

While effort has been made to accurately describe compounds in the accompanying sequence listing, should there be any discrepancies between a description in this specification and in the accompanying sequence listing, the description in the specification and not in the sequence listing is the accurate description.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or(S), as a or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C. in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Effect of 5-10-5 MOE Modified Oligonucleotides with Mixed PS/PO Internucleoside Linkages Complementary to a Human PCDH19 RNA In Vitro, Single Dose

Modified oligonucleotides complementary to human PCDH19 nucleic acid were designed and tested for their single dose effects on PCDH19 RNA in vitro. The modified oligonucleotides were tested in a series of experiments that had the same culture conditions.

The modified oligonucleotides in the table below are 5-10-5 MOE modified oligonucleotides with mixed PS/PO internucleoside linkages. The modified oligonucleotides are 20 nucleosides in length. The sugar motif for the modified oligonucleotides is (from 5′ to 3′): eeeeeddddddddddeeeee; wherein each “d” represents a 2′-β-D-deoxyribosyl sugar moiety, and each “e” represents a 2′-MOE ribosyl sugar moiety. The internucleoside linkage motif for the modified oligonucleotides is (from 5′ to 3′): soooossssssssssooss; wherein each “s” represents a phosphorothioate internucleoside linkage and each “o” represents a phosphodiester internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 1 (ENSEMBL Accession No. ENSG00000165194.15 from version 104: May 2021), to SEQ ID NO: 2 (the cDNA of ENSEMBL Accession No. ENST00000373034.8 from version 104: May2021), or to both. “N/A” indicates that the modified oligonucleotide is not 100% complementary to that particular target nucleic acid sequence.

SHSY5Y cells, plated at a density of 15,000 cells/well, were differentiated in Neurobasal media supplemented with B27 (ThermoFisher), penicillin/streptomycin (ThermoFisher), and 10 μM retinoic acid (Sigma) for 10 days. Differentiated SH-SY5Y cells were treated with modified oligonucleotide at a concentration of 15,000 nM by free uptake. After a treatment period of approximately 5 days, total RNA was isolated from the cells and PCDH19 RNA levels were measured by quantitative real-time RT-PCR using human primer-probe set RTS53390 (forward sequence GCTAACCACATCTACCATCACTC, designated herein as SEQ ID NO: 3; reverse sequence GCTATTCACGTAGTTGGAGTCA, designated herein as SEQ ID NO: 4; probe sequence TTTCAGTCTCAGGCAGAGGCACAC, designated herein as SEQ ID NO: 5). PCDH19 RNA levels were normalized to total RNA content, as measured by RIBOGREEN®. Reduction of PCDH19 RNA is presented in the table below as percent PCDH19 RNA relative to the amount in untreated control cells (% UTC).

Each separate experiment described in this example is identified by an Assay Identification letter in the table column labeled “AID”.

TABLE 1
Reduction of PCDH19 RNA by 5-10-5 MOE modified oligonucleotides with mixed PS/PO internucleoside
linkages at a concentration of 15,000 nM in differentiated SH-SY5Y cells

| | SEQ ID | SEQ ID | SEQ ID | SEQ | | | | |
|---|---|---|---|---|---|---|---|---|
| | NO: 1 | NO: 1 | NO: 2 | ID NO: | | | | |
| Compound | Start | Stop | Start | 2 Stop | | PCDH19 | | SEQ ID |
| No. | Site | Site | Site | Site | Sequence (5′ to 3′) | (% UTC) | AID | NO |
| 1549516 | 21259 | 21278 | N/A | N/A | GCTTCTTTATTAATTACCCA | 36 | A | 15 |
| 1549535 | 117328 | 117347 | 8454 | 8473 | ACTGCTATTATGTACAATAA | 74 | A | 16 |
| 1549542 | 60204 | 60223 | N/A | N/A | TTCTTTTTCTCTCTCATCTA | 17 | A | 17 |
| 1549557 | 52717 | 52736 | N/A | N/A | CCCACTATTTTCATCACATC | 27 | A | 18 |
| 1549558 | 41754 | 41773 | N/A | N/A | GCTTGTTTTCTTCCTCTCCT | 3 | A | 19 |
| 1549562 | 34367 | 34386 | N/A | N/A | GCTGTCACTTTTCCATACTA | 8 | A | 20 |
| 1549589 | 25522 | 25541 | N/A | N/A | ACATCATTTTTCATGTGGTC | 9 | A | 21 |
| 1549605 | 72604 | 72623 | N/A | N/A | ATTCTCTCTCATTTCCACCC | 9 | A | 22 |
| 1549612 | 18035 | 18054 | N/A | N/A | GTTAGCTTTCTTTCCATTTC | 27 | A | 23 |
| 1549617 | 84410 | 84429 | N/A | N/A | TTGTTTTTTTCATATCCCTA | 13 | A | 24 |
| 1549620 | 6722 | 6741 | 3936 | 3955 | CTTAGGCTCACTTTCTCCTC | 80 | A | 25 |
| 1549626 | 68244 | 68263 | 4397 | 4416 | GGTCACTCTCCTCATGTCCA | 10 | A | 26 |
| 1549632 | 59780 | 59799 | N/A | N/A | GTTTATTTACTTTTTTATGC | 52 | A | 27 |
| 1549640 | 80014 | 80033 | N/A | N/A | CATCATTCCTCTTTTTCCTC | 11 | A | 28 |
| 1549647 | 45631 | 45650 | N/A | N/A | GCTTAGTTATATCTCACCCC | 47 | A | 29 |
| 1549651 | 114228 | 114247 | 5354 | 5373 | TCTCTGTTTCCCCAACATCA | 11 | A | 30 |
| 1549652 | 11035 | 11054 | N/A | N/A | GCACTTATCATTCTATGCTA | 22 | A | 31 |
| 1549659 | 54750 | 54769 | N/A | N/A | TTTGATGCTTTTACTCTACT | 13 | A | 32 |
| 1549660 | 114366 | 114385 | 5492 | 5511 | CCAGAGCTTTTTTTTTTCAC | 27 | A | 33 |
| 1549665 | 29518 | 29537 | N/A | N/A | GCCTGCTTTCTGCTTCAATC | 52 | A | 34 |
| 1549672 | 19426 | 19445 | N/A | N/A | GCTCTTTTCTGATAACCTAT | 34 | A | 35 |
| 1549682 | 4951 | 4970 | N/A | N/A | GCATCATTCATTTTCAGCAA | 29 | A | 36 |
| 1549684 | 64160 | 64179 | N/A | N/A | CCATGTTTCTATTACTGCTC | 66 | A | 37 |
| 1549688 | 82577 | 82596 | N/A | N/A | ATTTTTATCTTATTTCATTC | 114 | A | 38 |
| 1549704 | 6274 | 6293 | N/A | N/A | ACTGTGCCTTATTCTCACTC | 11 | A | 39 |
| 1549705 | 30259 | 30278 | N/A | N/A | GCTGCTTCTTTCTTTACTTT | 18 | A | 40 |
| 1549708 | 82507 | 82526 | N/A | N/A | TTCTGATTTTGATCCTCACT | 62 | A | 41 |
| 1549720 | 39476 | 39495 | N/A | N/A | TGCATTTTTTCCTATAATCT | 38 | A | 42 |
| 1549724 | 95382 | 95401 | N/A | N/A | CATCTTGTCTCCATTCCATA | 31 | A | 43 |
| 1549725 | 93272 | 93291 | N/A | N/A | CTTTGTTTCCTACTGCCATA | 13 | A | 44 |
| 1549727 | 115538 | 115557 | 6664 | 6683 | TGACATATTTCTTTAGTTTA | 35 | A | 45 |
| 1549728 | 23346 | 23365 | N/A | N/A | TCATAGTCTCCATTTATCCA | 34 | A | 46 |
| 1549733 | 118288 | 118307 | 9414 | 9433 | GCATCCCTCTAAATTTCCAC | 14 | A | 47 |
| 1549735 | 98283 | 98302 | N/A | N/A | AGCAGTTTTAATCCTTCCTC | 59 | A | 48 |
| 1549740 | 33164 | 33183 | N/A | N/A | ATGTTTATTTCTCTATGCCT | 48 | A | 49 |
| 1549743 | 76485 | 76504 | N/A | N/A | GTTTGTTTTTTTCCTCTTCA | 12 | A | 50 |
| 1549750 | 95898 | 95917 | N/A | N/A | GCTTGCCTTTTTTTCTTCCC | 31 | A | 51 |
| 1549755 | 12335 | 12354 | N/A | N/A | CCTACTATCTTTCTTCTCCA | 11 | A | 52 |
| 1549764 | 55287 | 55306 | N/A | N/A | CTTTTTTATTTCATCTCAGT | 71 | A | 53 |
| 1549775 | 111784 | 111803 | N/A | N/A | ATGGACACTTTCATCCCCCT | 32 | A | 54 |
| 1549781 | 412 | 431 | 412 | 431 | CTTGATTTCATCTTCCCGGA | 20 | A | 55 |
| 1549782 | 64802 | 64821 | N/A | N/A | CTGCTTTGTCTTCACTTCTC | 48 | A | 56 |
| 1549814 | 12310 | 12329 | N/A | N/A | CTTCTTTTTCAACCTCCTTT | 103 | A | 57 |
| 1549821 | 43462 | 43481 | N/A | N/A | ACTTTATTAGTCTACTTCTT | 97 | A | 58 |
| 1549824 | 25784 | 25803 | N/A | N/A | CCTATTTATTCTTCCATACA | 88 | A | 59 |
| 1549834 | 78893 | 78912 | N/A | N/A | GAGTCTGTTTTATCTATCCA | 20 | A | 60 |
| 1549838 | 15892 | 15911 | N/A | N/A | GTGATTTTTCTCCCTCCCCA | 54 | A | 61 |
| 1549839 | 70900 | 70919 | N/A | N/A | TCTATTTTCCCTTCTCACCT | 86 | A | 62 |
| 1549841 | 4534 | 4553 | N/A | N/A | GCTATTTTTACTGAAACATC | 7 | A | 63 |
| 1549843 | 114008 | 114027 | 5134 | 5153 | GGTTTCTTTCTCTTCTTCCT | 33 | A | 64 |
| 1549851 | 43971 | 43990 | N/A | N/A | TTCTCATTTTTACTGCCTCA | 38 | A | 65 |
| 1549852 | 25734 | 25753 | N/A | N/A | ATATTCTCTTTTTCCCTCCC | 17 | A | 66 |
| 1549860 | 7476 | 7495 | 4018 | 4037 | GCGGATGTCATTCTTACTGA | 36 | A | 67 |
| 1549862 | 118347 | 118366 | 9473 | 9492 | AACAATTTTATCTTCATATA | 107 | A | 68 |
| 1549864 | 115868 | 115887 | 6994 | 7013 | GTTCTTTTTCAGAAATGTTC | 55 | A | 69 |
| 1549868 | 115178 | 115197 | 6304 | 6323 | ACTGCTTACTCTATCCCTAC | 68 | A | 70 |
| 1549873 | 52791 | 52810 | N/A | N/A | CCTTGTGTCATTTTTTTCTC | 26 | A | 71 |
| 1549876 | 12320 | 12339 | N/A | N/A | CTCCATTTTCCTTCTTTTTC | 30 | A | 72 |
| 1549880 | 17979 | 17998 | N/A | N/A | CTTCTTTTTCTATTTCTAAA | 120 | A | 73 |
| 1549884 | 38627 | 38646 | N/A | N/A | TGCCTTATTTTTCTCCATAT | 62 | A | 74 |
| 1549887 | 10648 | 10667 | N/A | N/A | GTGTCATTTTCCCTACGCAG | 1 | A | 75 |
| 1549895 | 25884 | 25903 | N/A | N/A | ACTCATTTTCCATCTATGAA | 32 | A | 76 |
| 1549896 | 49308 | 49327 | N/A | N/A | GCTTCTCATTTTATTTGGGA | 33 | A | 77 |
| 1549908 | 20421 | 20440 | N/A | N/A | GCTGGTATTTTTCTTTGTGC | 64 | A | 78 |
| 1549910 | 13019 | 13038 | N/A | N/A | CTGATGTTTTATTTTCATTA | 62 | A | 79 |
| 1549911 | 36819 | 36838 | N/A | N/A | GCTTCAGTTTTTAACCTCTC | 11 | A | 80 |
| 1549912 | 63480 | 63499 | N/A | N/A | GTTCATTTCCTATTTCATTT | 27 | A | 81 |
| 1549915 | 115316 | 115335 | 6442 | 6461 | GTTCACATTTTTAATCAAAA | 6 | A | 82 |
| 1549918 | 107500 | 107519 | N/A | N/A | TCTATATTTTCTGCTACCTT | 88 | A | 83 |
| 1549923 | 47992 | 48011 | N/A | N/A | GTACACCCTTTCCTCATCCT | 65 | A | 84 |
| 1549930 | 117940 | 117959 | 9066 | 9085 | GCATCATCAGTTTTCCCTAA | 54 | A | 85 |
| 1549937 | 85958 | 85977 | N/A | N/A | ACTTGTTTGATTTTCATCCT | 11 | A | 86 |
| 1549944 | 73355 | 73374 | N/A | N/A | CCAATCTCATATCAGGATTA | 55 | A | 87 |
| | 75767 | 75786 | | | | | | |
| 1549950 | 72467 | 72486 | N/A | N/A | AAGAGGATCTTAAAAGCTAT | 26 | A | 88 |
| | 75978 | 75997 | | | | | | |
| 1549973 | 73148 | 73167 | N/A | N/A | CTTGTCAATCACTCTTTTTA | 14 | A | 89 |
| | 75560 | 75579 | | | | | | |
| 1549980 | 73316 | 73335 | N/A | N/A | TGGGATATCCACATCATCAA | 4 | A | 90 |
| | 75728 | 75747 | | | | | | |
| 1549990 | 73218 | 73237 | N/A | N/A | AAATTCAAGCCCCATGATAC | 152 | A | 91 |
| | 75630 | 75649 | | | | | | |
| 1549991 | 73210 | 73229 | N/A | N/A | GCCCCATGATACCCCTAATC | 39 | A | 92 |
| | 75622 | 75641 | | | | | | |
| 1549514 | 84411 | 84430 | N/A | N/A | GTTGTTTTTTTCATATCCCT | 32 | B | 93 |
| 1549520 | 17980 | 17999 | N/A | N/A | ACTTCTTTTTCTATTTCTAA | 10 | B | 94 |
| 1549526 | 59851 | 59870 | N/A | N/A | CGTACATTTTCATACCAAGA | 10 | B | 95 |
| 1549532 | 118348 | 118367 | 9474 | 9493 | CAACAATTTTATCTTCATAT | 59 | B | 96 |
| 1549538 | 114367 | 114386 | 5493 | 5512 | TCCAGAGCTTTTTTTTTTCA | 71 | B | 97 |
| 1549543 | 95396 | 95415 | N/A | N/A | CCCACTCCTCTTCTCATCTT | 165 | B | 98 |
| 1549546 | 46229 | 46248 | N/A | N/A | CCATTTTCTTCCTTCATCAT | 25 | B | 99 |
| 1549553 | 87014 | 87033 | N/A | N/A | ACCAGCTATTTCCTTCCATC | 53 | B | 100 |
| 1549569 | 78959 | 78978 | N/A | N/A | GCTTTATATTCTCTCTACCT | 62 | B | 101 |
| 1549577 | 31478 | 31497 | N/A | N/A | CTGCACACTTCTTCACTCCC | 21 | B | 102 |
| 1549582 | 82580 | 82599 | N/A | N/A | GATATTTTTATCTTATTICA | 102 | B | 103 |
| 1549586 | 52767 | 52786 | N/A | N/A | GCATTTGTTCTACCTCATTC | 91 | B | 104 |
| 1549591 | 38630 | 38649 | N/A | N/A | TTCTGCCTTATTTTTCTCCA | 50 | B | 105 |
| 1549594 | 27059 | 27078 | N/A | N/A | ACTCACATTGAATCCTCACA | 51 | B | 106 |
| 1549601 | 13020 | 13039 | N/A | N/A | ACTGATGTTTTATTTTCATT | 75 | B | 107 |
| 1549604 | 12311 | 12330 | N/A | N/A | CCTTCTTTTTCAACCTCCTT | 43 | B | 108 |
| 1549610 | 114009 | 114028 | 5135 | 5154 | TGGTTTCTTTCTCTTCTTCC | 26 | B | 109 |
| 1549611 | 112143 | 112162 | N/A | N/A | CTTCCTTGTATACTTGTATT | 50 | B | 110 |
| 1549613 | 34370 | 34389 | N/A | N/A | GTTGCTGTCACTTTTCCATA | 5 | B | 111 |
| 1549618 | 60582 | 60601 | N/A | N/A | GTTTCATTTCTATTATTTTA | 58 | B | 112 |
| 1549629 | 39477 | 39496 | N/A | N/A | ATGCATTTTTTCCTATAATC | 30 | B | 113 |
| 1549631 | 18217 | 18236 | N/A | N/A | GTACGGATTTTTCTCCACCT | 135 | B | 114 |
| 1549643 | 25735 | 25754 | N/A | N/A | TATATTCTCTTTTTCCCTCC | 26 | B | 115 |
| 1549653 | 70901 | 70920 | N/A | N/A | TTCTATTTTCCCTTCTCACC | 17 | B | 116 |
| 1549654 | 21405 | 21424 | N/A | N/A | ACTTTTGTTTTACACATTCC | 12 | B | 117 |
| 1549655 | 76652 | 76671 | N/A | N/A | GTGCTCCTTTTACATATCCT | 29 | B | 118 |
| 1549664 | 43972 | 43991 | N/A | N/A | TTTCTCATTTTTACTGCCTC | 32 | B | 119 |
| 1549675 | 51765 | 51784 | N/A | N/A | GCACTTTTCTTTGCTCTCAT | 114 | B | 120 |
| 1549679 | 10719 | 10738 | N/A | N/A | GCTCTTCTATAGATTTCATT | 78 | B | 121 |
| 1549687 | 33294 | 33313 | N/A | N/A | ACTTTTGGTTCCCTCTCTTT | 142 | B | 122 |
| 1549691 | 5397 | 5416 | N/A | N/A | CCCACATTTTTCTCCTATAA | 18 | B | 123 |
| 1549700 | 7441 | 7460 | 3983 | 4002 | TTGATTTCTTTTGATGCCCA | 6 | B | 124 |
| 1549706 | 6393 | 6412 | N/A | N/A | GTCATTTATTACTTTGCACA | 23 | B | 125 |
| 1549721 | 15893 | 15912 | N/A | N/A | AGTGATTTTTCTCCCTCCCC | 112 | B | 126 |
| 1549723 | 11811 | 11830 | N/A | N/A | GCTATCTTCCATTCAGCACT | 84 | B | 127 |
| 1549730 | 64162 | 64181 | N/A | N/A | GCCCATGTTTCTATTACTGC | 13 | B | 128 |
| 1549736 | 12323 | 12342 | N/A | N/A | CTTCTCCATTTTCCTTCTTT | 35 | B | 129 |
| 1549737 | 25785 | 25804 | N/A | N/A | TCCTATTTATTCTTCCATAC | 12 | B | 130 |
| 1549742 | 68853 | 68872 | N/A | N/A | GTTTGGTATTTTCTATCCTC | 42 | B | 131 |
| 1549744 | 4743 | 4762 | N/A | N/A | GTTTCTTCTTTTCTCCCTTT | 12 | B | 132 |
| 1549754 | 63481 | 63500 | N/A | N/A | TGTTCATTTCCTATTTCATT | 89 | B | 133 |
| 1549758 | 12547 | 12566 | N/A | N/A | TGCATTTTCTTCCTCCAATA | 38 | B | 134 |
| 1549762 | 20492 | 20511 | N/A | N/A | GCTTATTTTTTAATTTAGTT | 76 | B | 135 |
| 1549763 | 80015 | 80034 | N/A | N/A | ACATCATTCCTCTTTTTCCT | 34 | B | 136 |
| 1549765 | 64905 | 64924 | N/A | N/A | GTTAATTTCTTCTAACATTA | 75 | B | 137 |
| 1549771 | 415 | 434 | 415 | 434 | CGTCTTGATTTCATCTTCCC | 42 | B | 138 |
| 1549772 | 20106 | 20125 | N/A | N/A | ACTTATGCTTTCTCTATGTC | 19 | B | 139 |
| 1549783 | 93274 | 93293 | N/A | N/A | TTCTTTGTTTCCTACTGCCA | 38 | B | 140 |
| 1549785 | 117943 | 117962 | 9069 | 9088 | TCAGCATCATCAGTTTTCCC | 53 | B | 141 |
| 1549799 | 118292 | 118311 | 9418 | 9437 | ACTTGCATCCCTCTAAATTT | 99 | B | 142 |
| 1549804 | 41756 | 41775 | N/A | N/A | ATGCTTGTTTTCTTCCTCTC | 6 | B | 143 |
| 1549810 | 29593 | 29612 | N/A | N/A | TCTGATTCTTATTCCTCCCA | 72 | B | 144 |
| 1549827 | 38616 | 38635 | N/A | N/A | TCTCCATATCACTTATCTTC | 84 | B | 145 |
| 1549828 | 107642 | 107661 | N/A | N/A | GTCACCTTTTCATTTATCCA | 121 | B | 146 |
| 1549833 | 43468 | 43487 | N/A | N/A | GCACAGACTTTATTAGTCTA | 21 | B | 147 |
| 1549840 | 7508 | 7527 | 4050 | 4069 | TTGTCTGTCTCCTCCACATC | 95 | B | 148 |
| 1549846 | 56043 | 56062 | N/A | N/A | GTATATTTTTTCACACTTCT | 77 | B | 149 |
| 1549853 | 115539 | 115558 | 6665 | 6684 | GTGACATATTTCTTTAGTTT | 39 | B | 150 |
| 1549867 | 25672 | 25691 | N/A | N/A | TCTTCCTTTTATTTAGTTAA | 70 | B | 151 |
| 1549871 | 106274 | 106293 | N/A | N/A | TCCACATTTTTCATTCATCT | 47 | B | 152 |
| 1549879 | 23738 | 23757 | N/A | N/A | CTGCTTTATCACATTAACTT | 51 | B | 153 |
| 1549882 | 96204 | 96223 | N/A | N/A | GTTTATATTTTTAACACCCT | 131 | B | 154 |
| 1549900 | 114229 | 114248 | 5355 | 5374 | GTCTCTGTTTCCCCAACATC | 66 | B | 155 |
| 1549901 | 117464 | 117483 | 8590 | 8609 | ATGCCAGTTTTTCAAAGTCA | 15 | B | 156 |
| 1549909 | 72607 | 72626 | N/A | N/A | GTCATTCTCTCTCATTTCCA | 92 | B | 157 |
| 1549913 | 54769 | 54788 | N/A | N/A | GCTTTTCTTCCATTTCAAGT | 93 | B | 158 |
| 1549924 | 115944 | 115963 | 7070 | 7089 | GCCACACCAATATTTTGCAT | 66 | B | 159 |
| 1549931 | 82561 | 82580 | N/A | N/A | ATTCTCTCTTATCACCTCAT | 28 | B | 160 |
| 1549932 | 115179 | 115198 | 6305 | 6324 | TACTGCTTACTCTATCCCTA | 27 | B | 161 |
| 1549934 | 48128 | 48147 | N/A | N/A | ATGTGTCCTTTCCTTTCCCC | 49 | B | 162 |
| 1549939 | 53228 | 53247 | N/A | N/A | ATTCATTTATCTATCCAGTA | 144 | B | 163 |
| 1549940 | 115317 | 115336 | 6443 | 6462 | TGTTCACATTTTTAATCAAA | 56 | B | 164 |
| 1549945 | 73219 | 73238 | N/A | N/A | GAAATTCAAGCCCCATGATA | 227 | B | 165 |
| | 75631 | 75650 | | | | | | |
| 1549948 | 72468 | 72487 | N/A | N/A | AAAGAGGATCTTAAAAGCTA | 17 | B | 166 |
| | 75979 | 75998 | | | | | | |
| 1549977 | 73317 | 73336 | N/A | N/A | ATGGGATATCCACATCATCA | 12 | B | 167 |
| | 75729 | 75748 | | | | | | |
| 1549988 | 73211 | 73230 | N/A | N/A | AGCCCCATGATACCCCTAAT | 328 | B | 168 |
| | 75623 | 75642 | | | | | | |
| 1550004 | 73149 | 73168 | N/A | N/A | ACTTGTCAATCACTCTTTTT | 107 | B | 169 |
| | 75561 | 75580 | | | | | | |
| 1550006 | 73356 | 73375 | N/A | N/A | CCCAATCTCATATCAGGATT | 30 | B | 170 |
| | 75768 | 75787 | | | | | | |
| 1549513 | 43411 | 43430 | N/A | N/A | GCATTTCTTTTCTACCAGTA | 6 | C | 171 |
| 1549525 | 48667 | 48686 | N/A | N/A | TTGCCTTTTCTTTTTCTCAA | 29 | C | 172 |
| 1549533 | 114010 | 114029 | 5136 | 5155 | GTGGTTTCTTTCTCTTCTTC | 50 | C | 173 |
| 1549537 | 46230 | 46249 | N/A | N/A | CCCATTTTCTTCCTTCATCA | 37 | C | 174 |
| 1549540 | 117980 | 117999 | 9106 | 9125 | AGGTCATCTTGAATTGCCAA | 66 | C | 175 |
| 1549541 | 118295 | 118314 | 9421 | 9440 | TGTACTTGCATCCCTCTAAA | 64 | C | 176 |
| 1549544 | 21079 | 21098 | N/A | N/A | CATACTTCCTTATTTTTCTT | 39 | C | 177 |
| 1549547 | 112166 | 112185 | N/A | N/A | GCATCAGTCTATATATAACA | 62 | C | 178 |
| 1549552 | 93360 | 93379 | N/A | N/A | GCTACAATTTCAATAGATAT | 71 | C | 179 |
| 1549556 | 52221 | 52240 | N/A | N/A | GTTCACTGTTATCACTCCCA | 46 | C | 180 |
| 1549559 | 38646 | 38665 | N/A | N/A | ACTCATTATCTTCCTTTTCT | 46 | C | 181 |
| 1549560 | 38617 | 38636 | N/A | N/A | TTCTCCATATCACTTATCTT | 97 | C | 182 |
| 1549564 | 12312 | 12331 | N/A | N/A | TCCTTCTTTTTCAACCTCCT | 26 | C | 183 |
| 1549584 | 20160 | 20179 | N/A | N/A | CTTAACTTCTATCTTGTCTC | 50 | C | 184 |
| 1549585 | 78961 | 78980 | N/A | N/A | CTGCTTTATATTCTCTCTAC | 96 | C | 185 |
| 1549587 | 87648 | 87667 | N/A | N/A | GTATTGTTTTTTCTAATCCT | 40 | C | 186 |
| 1549588 | 29869 | 29888 | N/A | N/A | GCTCTTTTCTTTCATAGCAT | 35 | C | 187 |
| 1549596 | 76868 | 76887 | N/A | N/A | GCATAATTTTATCTAGCATA | 18 | C | 188 |
| 1549609 | 6716 | 6735 | 3930 | 3949 | CTCACTTTCTCCTCTGTCTT | 49 | C | 189 |
| 1549614 | 36674 | 36693 | N/A | N/A | GCCTATTTTCCTTTCTTCGA | 36 | C | 190 |
| 1549635 | 107970 | 107989 | N/A | N/A | GCATCTTTATTACTAGCGGT | 52 | C | 191 |
| 1549636 | 33582 | 33601 | N/A | N/A | CTTAGATTTTTTTCTTTATC | 122 | C | 192 |
| 1549637 | 106275 | 106294 | N/A | N/A | TTCCACATTTTTCATTCATC | 90 | C | 193 |
| 1549639 | 96731 | 96750 | N/A | N/A | AGTACTTTCTTATCCCCTTA | 50 | C | 194 |
| 1549644 | 44572 | 44591 | N/A | N/A | TCTATTATCTCTCTTGCACA | 49 | C | 195 |
| 1549650 | 7446 | 7465 | 3988 | 4007 | CTTGCTTGATTTCTTTTGAT | 76 | C | 196 |
| 1549662 | 1369 | 1388 | 1369 | 1388 | ACTCAGTTTTACCCCTTCAA | 33 | C | 197 |
| 1549667 | 40586 | 40605 | N/A | N/A | ACTTTTTGCCTCTCTCATAA | 86 | C | 198 |
| 1549677 | 114230 | 114249 | 5356 | 5375 | TGTCTCTGTTTCCCCAACAT | 26 | C | 199 |
| 1549680 | 70902 | 70921 | N/A | N/A | ATTCTATTTTCCCTTCTCAC | 94 | C | 200 |
| 1549683 | 82980 | 82999 | N/A | N/A | ATTTCTATCATTACTCCCAT | 52 | C | 201 |
| 1549686 | 63487 | 63506 | N/A | N/A | GTGCTTTGTTCATTTCCTAT | 97 | C | 202 |
| 1549693 | 118350 | 118369 | 9476 | 9495 | TACAACAATTTTATCTTCAT | 86 | C | 203 |
| 1549694 | 12327 | 12346 | N/A | N/A | CTTTCTTCTCCATTTTCCTT | 32 | C | 204 |
| 1549696 | 13041 | 13060 | N/A | N/A | GCTATTTTTTTTCAAAGCCT | 80 | C | 205 |
| 1549716 | 32171 | 32190 | N/A | N/A | TTGTTCATTTATACCACACA | 32 | C | 206 |
| 1549719 | 115679 | 115698 | 6805 | 6824 | CCTCTTTTGCATTCTAGTGC | 54 | C | 207 |
| 1549729 | 82564 | 82583 | N/A | N/A | TTCATTCTCTCTTATCACCT | 39 | C | 208 |
| 1549734 | 53934 | 53953 | N/A | N/A | CTTCTTTTTTTAACTGCTGA | 39 | C | 209 |
| 1549738 | 72609 | 72628 | N/A | N/A | CTGTCATTCTCTCTCATTTC | 72 | C | 210 |
| 1549746 | 95645 | 95664 | N/A | N/A | CCATCTGTTCTATCCGCTTT | 69 | C | 211 |
| 1549747 | 64957 | 64976 | N/A | N/A | ACACTTCTATATCTTCTGTA | 53 | C | 212 |
| 1549766 | 25685 | 25704 | N/A | N/A | GCTTTTTTTTGAATCTTCCT | 17 | C | 213 |
| 1549773 | 43528 | 43547 | N/A | N/A | CATCATTCTTATTTATTCAT | 47 | C | 214 |
| 1549777 | 25786 | 25805 | N/A | N/A | TTCCTATTTATTCTTCCATA | 74 | C | 215 |
| 1549784 | 80720 | 80739 | N/A | N/A | GCATCAGTTTTTCTTCTAAA | 8 | C | 216 |
| 1549786 | 25736 | 25755 | N/A | N/A | ATATATTCTCTTTTTCCCTC | 79 | C | 217 |
| 1549790 | 84412 | 84431 | N/A | N/A | AGTTGTTTTTTTCATATCCC | 23 | C | 218 |
| 1549796 | 18277 | 18296 | N/A | N/A | ACATTTATTTGATCCCATAT | 81 | C | 219 |
| 1549803 | 15925 | 15944 | N/A | N/A | ACTCTCATTCTCATTTTTTC | 61 | C | 220 |
| 1549812 | 114463 | 114482 | 5589 | 5608 | GCTCAATTTCACATCACTGA | 74 | C | 221 |
| 1549819 | 5398 | 5417 | N/A | N/A | CCCCACATTTTTCTCCTATA | 20 | C | 222 |
| 1549820 | 17981 | 18000 | N/A | N/A | CACTTCTTTTTCTATTTCTA | 35 | C | 223 |
| 1549822 | 8230 | 8249 | N/A | N/A | CTATCCATATTCTCTCACCA | 54 | C | 224 |
| 1549829 | 115181 | 115200 | 6307 | 6326 | TGTACTGCTTACTCTATCCC | 67 | C | 225 |
| 1549831 | 21617 | 21636 | N/A | N/A | GTGTATTGCCAAATTGCCCC | 27 | C | 226 |
| 1549835 | 23741 | 23760 | N/A | N/A | ACTCTGCTTTATCACATTAA | 24 | C | 227 |
| 1549855 | 4745 | 4764 | N/A | N/A | GTGTTTCTTCTTTTCTCCCT | 13 | C | 228 |
| 1549856 | 28043 | 28062 | N/A | N/A | GCTTTTGTTCTCCCCCTCCC | 32 | C | 229 |
| 1549861 | 12548 | 12567 | N/A | N/A | TTGCATTTTCTTCCTCCAAT | 12 | C | 230 |
| 1549863 | 11921 | 11940 | N/A | N/A | TGTTCCATTCCCTTTACCTT | 19 | C | 231 |
| 1549870 | 54873 | 54892 | N/A | N/A | CTGTGGTTTTTTTCTTGTCT | 33 | C | 232 |
| 1549872 | 116325 | 116344 | 7451 | 7470 | GTTCCCACTTTCCACAATCT | 28 | C | 233 |
| 1549874 | 115319 | 115338 | 6445 | 6464 | CTTGTTCACATTTTTAATCA | 23 | C | 234 |
| 1549891 | 117478 | 117497 | 8604 | 8623 | TCTTAAGCTCATTTATGCCA | 50 | C | 235 |
| 1549916 | 69476 | 69495 | N/A | N/A | GTACATTTTTTCCTTTGGAC | 17 | C | 236 |
| 1549919 | 59998 | 60017 | N/A | N/A | TATATTTCTAATATCACATT | 145 | C | 237 |
| 1549922 | 52784 | 52803 | N/A | N/A | TCATTTTTTTCTCACCTGCA | 57 | C | 238 |
| 1549926 | 56190 | 56209 | N/A | N/A | GCCTCATTAGTACTACATTC | 75 | C | 239 |
| 1549935 | 64264 | 64283 | N/A | N/A | CTTGCTTTATTACCTGCATA | 120 | C | 240 |
| 1549938 | 10817 | 10836 | N/A | N/A | CTTGTCTTTTTCATCTCTCT | 2 | C | 241 |
| 1549943 | 60699 | 60718 | N/A | N/A | TTTATTCCTTGAATTCTACT | 81 | C | 242 |
| 1549946 | 73213 | 73232 | N/A | N/A | CAAGCCCCATGATACCCCTA | 58 | C | 243 |
| | 75625 | 75644 | | | | | | |
| 1549951 | 72469 | 72488 | N/A | N/A | AAAAGAGGATCTTAAAAGCT | 102 | C | 244 |
| | 75980 | 75999 | | | | | | |
| 1549965 | 73357 | 73376 | N/A | N/A | CCCCAATCTCATATCAGGAT | 31 | C | 245 |
| | 75769 | 75788 | | | | | | |
| 1549985 | 73312 | 73331 | N/A | N/A | ATATCCACATCATCAAAAAT | 100 | C | 246 |
| | 75724 | 75743 | | | | | | |
| 1550005 | 73318 | 73337 | N/A | N/A | AATGGGATATCCACATCATC | 25 | C | 247 |
| | 75730 | 75749 | | | | | | |
| 1550010 | 73150 | 73169 | N/A | N/A | TACTTGTCAATCACTCTTTT | 61 | C | 248 |
| | 75562 | 75581 | | | | | | |
| 1549515 | 46231 | 46250 | N/A | N/A | ACCCATTTTCTTCCTTCATC | 47 | D | 249 |
| 1549517 | 21747 | 21766 | N/A | N/A | TCCGATGTTTTATCCTCATA | 2 | D | 250 |
| 1549518 | 5510 | 5529 | N/A | N/A | GCTGATTTCTTTTTCATCAA | 7 | D | 251 |
| 1549527 | 69709 | 69728 | N/A | N/A | ACTGTTTAATTTTTTTCATC | 111 | D | 252 |
| 1549549 | 25687 | 25706 | N/A | N/A | ACGCTTTTTTTTGAATCTTC | 78 | D | 253 |
| 1549550 | 29895 | 29914 | N/A | N/A | CCCATTTTACTCTTTCATAA | 65 | D | 254 |
| 1549551 | 59718 | 59737 | N/A | N/A | ATTCATTTTAAAATCCCCCA | 98 | D | 255 |
| 1549563 | 93856 | 93875 | N/A | N/A | CTTGACATTCATATCCAGTA | 71 | D | 256 |
| 1549575 | 118146 | 118165 | 9272 | 9291 | GCTGATTTCTAGCTCGAGCT | 83 | D | 257 |
| 1549576 | 77602 | 77621 | N/A | N/A | GTTTAGATTTATCATTTCCC | 43 | D | 258 |
| 1549578 | 52552 | 52571 | N/A | N/A | CTTAGCATTTTCTCAGCACT | 53 | D | 259 |
| 1549590 | 25787 | 25806 | N/A | N/A | TTTCCTATTTATTCTTCCAT | 67 | D | 260 |
| 1549593 | 38943 | 38962 | N/A | N/A | TCTACTACTTCCATCACACT | 92 | D | 261 |
| 1549599 | 95893 | 95912 | N/A | N/A | CCTTTTTTTCTTCCCCACTC | 69 | D | 262 |
| 1549602 | 16836 | 16855 | N/A | N/A | ATGTTCATTTTATTCCCTCC | 17 | D | 263 |
| 1549619 | 33728 | 33747 | N/A | N/A | ACTATTTTCTTTTTAAATCA | 146 | D | 264 |
| 1549625 | 12549 | 12568 | N/A | N/A | GTTGCATTTTCTTCCTCCAA | 23 | D | 265 |
| 1549627 | 70908 | 70927 | N/A | N/A | GTGCAGATTCTATTTTCCCT | 30 | D | 266 |
| 1549628 | 17984 | 18003 | N/A | N/A | GCACACTTCTTTTTCTATTT | 6 | D | 267 |
| 1549634 | 12281 | 12300 | N/A | N/A | TCACTCTTTTCTACGCCCTC | 62 | D | 268 |
| 1549641 | 115711 | 115730 | 6837 | 6856 | TGACTTATCTACCTAAACTT | 122 | D | 269 |
| 1549658 | 6717 | 6736 | 3931 | 3950 | GCTCACTTTCTCCTCTGTCT | 71 | D | 270 |
| 1549669 | 23833 | 23852 | N/A | N/A | TTCGATCTTCTTATCACCTT | 36 | D | 271 |
| 1549670 | 48669 | 48688 | N/A | N/A | ACTTGCCTTTTCTTTTTCTC | 47 | D | 272 |
| 1549674 | 117686 | 117705 | 8812 | 8831 | CTGGAATATTTCATTATCAA | 30 | D | 273 |
| 1549676 | 115185 | 115204 | 6311 | 6330 | ACTTTGTACTGCTTACTCTA | 59 | D | 274 |
| 1549690 | 32172 | 32191 | N/A | N/A | GTTGTTCATTTATACCACAC | 69 | D | 275 |
| 1549692 | 115320 | 115339 | 6446 | 6465 | TCTTGTTCACATTTTTAATC | 29 | D | 276 |
| 1549695 | 116331 | 116350 | 7457 | 7476 | ATTAATGTTCCCACTTTCCA | 84 | D | 277 |
| 1549699 | 12331 | 12350 | N/A | N/A | CTATCTTTCTTCTCCATTTT | 120 | D | 278 |
| 1549707 | 114258 | 114277 | 5384 | 5403 | AATACATAATACTTTTCACA | 71 | D | 279 |
| 1549717 | 87649 | 87668 | N/A | N/A | AGTATTGTTTTTTCTAATCC | 84 | D | 280 |
| 1549732 | 18279 | 18298 | N/A | N/A | ACACATTTATTTGATCCCAT | 24 | D | 281 |
| 1549741 | 25737 | 25756 | N/A | N/A | TATATATTCTCTTTTTCCCT | 101 | D | 282 |
| 1549745 | 78962 | 78981 | N/A | N/A | ACTGCTTTATATTCTCTCTA | 53 | D | 283 |
| 1549749 | 4746 | 4765 | N/A | N/A | TGTGTTTCTTCTTTTCTCCC | 19 | D | 284 |
| 1549756 | 43413 | 43432 | N/A | N/A | TTGCATTTCTTTTCTACCAG | 16 | D | 285 |
| 1549760 | 13364 | 13383 | N/A | N/A | GCATTGTTTCTCATTGCTTT | 107 | D | 286 |
| 1549767 | 85361 | 85380 | N/A | N/A | ACTACCAGTATCCTCCATTC | 87 | D | 287 |
| 1549768 | 60704 | 60723 | N/A | N/A | GCTTGTTTATTCCTTGAATT | 45 | D | 288 |
| 1549776 | 1998 | 2017 | 1998 | 2017 | ACGCAGATTTCCATTGAGCT | 40 | D | 289 |
| 1549778 | 118458 | 118477 | 9584 | 9603 | CTGCTTTATCATCTTCATAA | 33 | D | 290 |
| 1549788 | 28085 | 28104 | N/A | N/A | ACACATTTCACATTCAGGTT | 12 | D | 291 |
| 1549789 | 114011 | 114030 | 5137 | 5156 | TGTGGTTTCTTTCTCTTCTT | 40 | D | 292 |
| 1549792 | 43529 | 43548 | N/A | N/A | ACATCATTCTTATTTATTCA | 59 | D | 293 |
| 1549806 | 97116 | 97135 | N/A | N/A | GATCATGTATCTTTACACTC | 95 | D | 294 |
| 1549807 | 114609 | 114628 | 5735 | 5754 | GCCTTTTGCCATCACCACAC | 69 | D | 295 |
| 1549809 | 55222 | 55241 | N/A | N/A | ATTCTTTTTCTAATTCGAAT | 107 | D | 296 |
| 1549811 | 20192 | 20211 | N/A | N/A | TCTGCTTTTCTCATTGACTT | 30 | D | 297 |
| 1549816 | 64669 | 64688 | N/A | N/A | TCATCTTTTTCATAGGCATC | 33 | D | 298 |
| 1549817 | 82565 | 82584 | N/A | N/A | TTTCATTCTCTCTTATCACC | 127 | D | 299 |
| 1549818 | 106548 | 106567 | N/A | N/A | CTACTTGTCTCTTTCTCACT | 159 | D | 300 |
| 1549825 | 59999 | 60018 | N/A | N/A | GTATATTTCTAATATCACAT | 183 | D | 301 |
| 1549845 | 12315 | 12334 | N/A | N/A | TTTTCCTTCTTTTTCAACCT | 69 | D | 302 |
| 1549848 | 113900 | 113919 | 5026 | 5045 | ATGCATGACTTTCTCGCTAT | 85 | D | 303 |
| 1549859 | 7460 | 7479 | 4002 | 4021 | CTGATTTTTTTCTTCTTGCT | 17 | D | 304 |
| 1549865 | 8975 | 8994 | N/A | N/A | CCATCATCCATATACCACTC | 47 | D | 305 |
| 1549878 | 82447 | 82466 | N/A | N/A | ATGCTTGTCTCTCCCTTTCA | 51 | D | 306 |
| 1549883 | 108229 | 108248 | N/A | N/A | TTGTCTTTATTCCTTCCTGC | 57 | D | 307 |
| 1549886 | 21083 | 21102 | N/A | N/A | GCTTCATACTTCCTTATTTT | 33 | D | 308 |
| 1549888 | 83231 | 83250 | N/A | N/A | AATGCATTTTCTCTCCCCAT | 32 | D | 309 |
| 1549889 | 74722 | 74741 | N/A | N/A | CAGTCTTTTCATATTGCTTC | 21 | D | 310 |
| 1549894 | 118314 | 118333 | 9440 | 9459 | ACTGCTTAATGAGACATACT | 64 | D | 311 |
| 1549897 | 53937 | 53956 | N/A | N/A | CTGCTTCTTTTTTTAACTGC | 50 | D | 312 |
| 1549899 | 52785 | 52804 | N/A | N/A | GTCATTTTTTTCTCACCTGC | 20 | D | 313 |
| 1549907 | 63654 | 63673 | N/A | N/A | CTTCCATTTCTCTCATGCAT | 92 | D | 314 |
| 1549914 | 65069 | 65088 | N/A | N/A | GTTATTGTTTAATACCATCA | 23 | D | 315 |
| 1549925 | 36678 | 36697 | N/A | N/A | GCTAGCCTATTTTCCTTTCT | 71 | D | 316 |
| 1549927 | 38624 | 38643 | N/A | N/A | CTTATTTTTCTCCATATCAC | 74 | D | 317 |
| 1549928 | 40644 | 40663 | N/A | N/A | TCTCTTCATTTCAACACATT | 131 | D | 318 |
| 1549929 | 45391 | 45410 | N/A | N/A | ATTTCTTTCAACATTTCCCA | 80 | D | 319 |
| 1549941 | 10818 | 10837 | N/A | N/A | TCTTGTCTTTTTCATCTCTC | 6 | D | 320 |
| 1549952 | 73319 | 73338 | N/A | N/A | TAATGGGATATCCACATCAT | 91 | D | 321 |
| | 75731 | 75750 | | | | | | |
| 1549964 | 73358 | 73377 | N/A | N/A | ACCCCAATCTCATATCAGGA | 53 | D | 322 |
| | 75770 | 75789 | | | | | | |
| 1549975 | 73151 | 73170 | N/A | N/A | CTACTTGTCAATCACTCTTT | 86 | D | 323 |
| | 75563 | 75582 | | | | | | |
| 1549995 | 73313 | 73332 | N/A | N/A | GATATCCACATCATCAAAAA | 37 | D | 324 |
| | 75725 | 75744 | | | | | | |
| 1550000 | 73214 | 73233 | N/A | N/A | TCAAGCCCCATGATACCCCT | 50 | D | 325 |
| | 75626 | 75645 | | | | | | |
| 1550008 | 72479 | 72498 | N/A | N/A | AGATAGCTAAAAAAGAGGAT | 100 | D | 326 |
| | 75990 | 76009 | | | | | | |
| 1549511 | 52787 | 52806 | N/A | N/A | GTGTCATTTTTTTCTCACCT | 34 | E | 327 |
| 1549512 | 18969 | 18988 | N/A | N/A | GTGCATCTCCTATTCATCCC | 13 | E | 328 |
| 1549519 | 78891 | 78910 | N/A | N/A | GTCTGTTTTATCTATCCACT | 9 | E | 329 |
| 1549523 | 4747 | 4766 | N/A | N/A | CTGTGTTTCTTCTTTTCTCC | 15 | E | 330 |
| 1549524 | 115191 | 115210 | 6317 | 6336 | ATATACACTTTGTACTGCTT | 35 | E | 331 |
| 1549530 | 43414 | 43433 | N/A | N/A | ATTGCATTTCTTTTCTACCA | 55 | E | 332 |
| 1549534 | 12550 | 12569 | N/A | N/A | AGTTGCATTTTCTTCCTCCA | 8 | E | 333 |
| 1549536 | 115331 | 115350 | 6457 | 6476 | TTTTAGTATTTTCTTGTTCA | 40 | E | 334 |
| 1549539 | 15628 | 15647 | N/A | N/A | CTGTCATATTTTATTTCATT | 21 | E | 335 |
| 1549545 | 12332 | 12351 | N/A | N/A | ACTATCTTTCTTCTCCATTT | 104 | E | 336 |
| 1549548 | 36733 | 36752 | N/A | N/A | ACTTGGACTTTTATCCTTTA | 53 | E | 337 |
| 1549554 | 5751 | 5770 | N/A | N/A | ACCAATTATTTAATTATACT | 72 | E | 338 |
| 1549555 | 12316 | 12335 | N/A | N/A | ATTTTCCTTCTTTTTCAACC | 75 | E | 339 |
| 1549561 | 9742 | 9761 | N/A | N/A | CTCTCACTTTCTATTGCTGA | 45 | E | 340 |
| 1549566 | 32756 | 32775 | N/A | N/A | GCACTCATTTTTGTAAACTC | 5 | E | 341 |
| 1549567 | 24150 | 24169 | N/A | N/A | GTGTATCTATATCTCCCTAC | 15 | E | 342 |
| 1549570 | 4111 | 4130 | N/A | N/A | TCTCTGTTTTTCCTACCATA | 53 | E | 343 |
| 1549572 | 59762 | 59781 | N/A | N/A | GCTTTTCTCAATTTTCCAAA | 15 | E | 344 |
| 1549580 | 75143 | 75162 | N/A | N/A | TTTTAGTGATTATTTTTCCA | 66 | E | 345 |
| 1549592 | 93859 | 93878 | N/A | N/A | TGTCTTGACATTCATATCCA | 72 | E | 346 |
| 1549597 | 107051 | 107070 | N/A | N/A | GCACTATTATCAATCCCATC | 87 | E | 347 |
| 1549600 | 25690 | 25709 | N/A | N/A | CCTACGCTTTTTTTTGAATC | 133 | E | 348 |
| 1549606 | 54367 | 54386 | N/A | N/A | GTCATTTTGCATTACTTTTA | 21 | E | 349 |
| 1549615 | 52594 | 52613 | N/A | N/A | CCTCATTTCTAATATATCTC | 158 | E | 350 |
| 1549616 | 92012 | 92031 | N/A | N/A | GTGCCTTTTTTCCCTTCCAT | 32 | E | 351 |
| 1549621 | 29896 | 29915 | N/A | N/A | CCCCATTTTACTCTTTCATA | 54 | E | 352 |
| 1549622 | 60000 | 60019 | N/A | N/A | TGTATATTTCTAATATCACA | 85 | E | 353 |
| 1549623 | 109643 | 109662 | N/A | N/A | GTGTCTTTAATTTAAACATA | 98 | E | 354 |
| 1549633 | 79186 | 79205 | N/A | N/A | CCTATTTTACACACCATCCT | 174 | E | 355 |
| 1549638 | 45396 | 45415 | N/A | N/A | CTTGTATTTCTTTCAACATT | 53 | E | 356 |
| 1549645 | 70084 | 70103 | N/A | N/A | CTTATTATCCAATCTACACA | 105 | E | 357 |
| 1549648 | 113937 | 113956 | 5063 | 5082 | CTTTGTTGCGACCTTCCTTC | 114 | E | 358 |
| 1549649 | 118337 | 118356 | 9463 | 9482 | TCTTCATATAATCAGCAACA | 75 | E | 359 |
| 1549656 | 38625 | 38644 | N/A | N/A | CCTTATTTTTCTCCATATCA | 95 | E | 360 |
| 1549657 | 114012 | 114031 | 5138 | 5157 | GTGTGGTTTCTTTCTCTTCT | 54 | E | 361 |
| 1549663 | 118459 | 118478 | 9585 | 9604 | GCTGCTTTATCATCTTCATA | 73 | E | 362 |
| 1549668 | 46232 | 46251 | N/A | N/A | GACCCATTTTCTTCCTTCAT | 35 | E | 363 |
| 1549689 | 21089 | 21108 | N/A | N/A | ATGTATGCTTCATACTTCCT | 79 | E | 364 |
| 1549710 | 12282 | 12301 | N/A | N/A | TTCACTCTTTTCTACGCCCT | 33 | E | 365 |
| 1549711 | 7461 | 7480 | 4003 | 4022 | ACTGATTTTTTTCTTCTTGC | 31 | E | 366 |
| 1549714 | 84408 | 84427 | N/A | N/A | GTTTTTTTCATATCCCTAAT | 35 | E | 367 |
| 1549722 | 20323 | 20342 | N/A | N/A | ATGATTTTATTATTTGAGTT | 76 | E | 368 |
| 1549739 | 21748 | 21767 | N/A | N/A | TTCCGATGTTTTATCCTCAT | 7 | E | 369 |
| 1549752 | 18030 | 18049 | N/A | N/A | CTTTCTTTCCATTTCAGAAA | 128 | E | 370 |
| 1549753 | 34364 | 34383 | N/A | N/A | GTCACTTTTCCATACTAATA | 30 | E | 371 |
| 1549759 | 95894 | 95913 | N/A | N/A | GCCTTTTTTTCTTCCCCACT | 26 | E | 372 |
| 1549770 | 82566 | 82585 | N/A | N/A | ATTTCATTCTCTCTTATCAC | 89 | E | 373 |
| 1549774 | 118198 | 118217 | 9324 | 9343 | GCCTATTGCAACTAATACCT | 55 | E | 374 |
| 1549779 | 55252 | 55271 | N/A | N/A | CTTTCTTATTATTTAGTATA | 77 | E | 375 |
| 1549787 | 38950 | 38969 | N/A | N/A | AGTACTTTCTACTACTTCCA | 56 | E | 376 |
| 1549794 | 115712 | 115731 | 6838 | 6857 | CTGACTTATCTACCTAAACT | 79 | E | 377 |
| 1549795 | 117886 | 117905 | 9012 | 9031 | TTGTGGGTTATTCTTTACCA | 22 | E | 378 |
| 1549800 | 43531 | 43550 | N/A | N/A | GCACATCATTCTTATTTATT | 9 | E | 379 |
| 1549805 | 28829 | 28848 | N/A | N/A | GCTTTTTTCTCTGCTACTCA | 13 | E | 380 |
| 1549808 | 6718 | 6737 | 3932 | 3951 | GGCTCACTTTCTCCTCTGTC | 36 | E | 381 |
| 1549813 | 85694 | 85713 | N/A | N/A | CTCTCATTTTACATTTTTAA | 50 | E | 382 |
| 1549815 | 48821 | 48840 | N/A | N/A | GCTACATTTTCTCAGATCTC | 68 | E | 383 |
| 1549823 | 76482 | 76501 | N/A | N/A | TGTTTTTTTCCTCTTCATTT | 44 | E | 384 |
| 1549826 | 63655 | 63674 | N/A | N/A | TCTTCCATTTCTCTCATGCA | 63 | E | 385 |
| 1549830 | 40647 | 40666 | N/A | N/A | TAGTCTCTTCATTTCAACAC | 54 | E | 386 |
| 1549832 | 97202 | 97221 | N/A | N/A | ATGCATTTTCATTCACAAAT | 74 | E | 387 |
| 1549837 | 17353 | 17372 | N/A | N/A | CTGTCCATTCTTCTCACTTA | 79 | E | 388 |
| 1549844 | 114265 | 114284 | 5391 | 5410 | AACACTTAATACATAATACT | 81 | E | 389 |
| 1549849 | 25738 | 25757 | N/A | N/A | ATATATATTCTCTTTTTCCC | 86 | E | 390 |
| 1549866 | 10821 | 10840 | N/A | N/A | ATTTCTTGTCTTTTTCATCT | 9 | E | 391 |
| 1549881 | 67873 | 67892 | N/A | N/A | ACTTTTCTCTTCCTATAAAA | 68 | E | 392 |
| 1549885 | 64799 | 64818 | N/A | N/A | CTTTGTCTTCACTTCTCAAA | 112 | E | 393 |
| 1549892 | 60993 | 61012 | N/A | N/A | GGTTATTTTTTTCTTTCTAA | 32 | E | 394 |
| 1549902 | 114701 | 114720 | 5827 | 5846 | GCTCATTCATTTGCTGTCTC | 76 | E | 395 |
| 1549903 | 25788 | 25807 | N/A | N/A | ATTTCCTATTTATTCTTCCA | 52 | E | 396 |
| 1549933 | 70964 | 70983 | N/A | N/A | ACTAGTTTTTTTCTCCTCAA | 34 | E | 397 |
| 1549936 | 116789 | 116808 | 7915 | 7934 | CTTGTAGCTTTTCACCAGAC | 73 | E | 398 |
| 1549942 | 82505 | 82524 | N/A | N/A | CTGATTTTGATCCTCACTAT | 90 | E | 399 |
| 1549947 | 73215 | 73234 | N/A | N/A | TTCAAGCCCCATGATACCCC | 92 | E | 400 |
| | 75627 | 75646 | | | | | | |
| 1549966 | 73153 | 73172 | N/A | N/A | CTCTACTTGTCAATCACTCT | 43 | E | 401 |
| | 75565 | 75584 | | | | | | |
| 1549969 | 73361 | 73380 | N/A | N/A | TAAACCCCAATCTCATATCA | 97 | E | 402 |
| | 75773 | 75792 | | | | | | |
| 1549984 | 73344 | 73363 | N/A | N/A | TCAGGATTACATATTTAAGT | 35 | E | 403 |
| | 75756 | 75775 | | | | | | |
| 1549994 | 73314 | 73333 | N/A | N/A | GGATATCCACATCATCAAAA | 70 | E | 404 |
| | 75726 | 75745 | | | | | | |
| 1549521 | 43415 | 43434 | N/A | N/A | AATTGCATTTCTTTTCTACC | 54 | F | 405 |
| 1549522 | 61905 | 61924 | N/A | N/A | CACATTTTACATTTAACACA | 114 | F | 406 |
| 1549528 | 84409 | 84428 | N/A | N/A | TGTTTTTTTCATATCCCTAA | 28 | F | 407 |
| 1549529 | 25883 | 25902 | N/A | N/A | CTCATTTTCCATCTATGAAA | 61 | F | 408 |
| 1549531 | 10103 | 10122 | N/A | N/A | GCATCTTTCACCTCTATTGT | 12 | F | 409 |
| 1549565 | 78892 | 78911 | N/A | N/A | AGTCTGTTTTATCTATCCAC | 47 | F | 410 |
| 1549568 | 5888 | 5907 | N/A | N/A | GTTGATTTTCTATTCTAATC | 26 | F | 411 |
| 1549571 | 114741 | 114760 | 5867 | 5886 | GTGAGTATTCTCCAAGTACT | 72 | F | 412 |
| 1549573 | 21234 | 21253 | N/A | N/A | GTTCATTTTACATTTTTTAT | 44 | F | 413 |
| 1549574 | 64800 | 64819 | N/A | N/A | GCTTTGTCTTCACTTCTCAA | 35 | F | 414 |
| 1549579 | 49307 | 49326 | N/A | N/A | CTTCTCATTTTATTTGGGAA | 101 | F | 415 |
| 1549581 | 12319 | 12338 | N/A | N/A | TCCATTTTCCTTCTTTTTCA | 27 | F | 416 |
| 1549583 | 45397 | 45416 | N/A | N/A | ACTTGTATTTCTTTCAACAT | 78 | F | 417 |
| 1549595 | 36818 | 36837 | N/A | N/A | CTTCAGTTTTTAACCTCTCC | 58 | F | 418 |
| 1549598 | 118239 | 118258 | 9365 | 9384 | TCATCAGGCTCATTCCCCTC | 69 | F | 419 |
| 1549603 | 92926 | 92945 | N/A | N/A | GCTTCATTCTGAATTTAATC | 51 | F | 420 |
| 1549607 | 115536 | 115555 | 6662 | 6681 | ACATATTTCTTTAGTTTATT | 74 | F | 421 |
| 1549608 | 76483 | 76502 | N/A | N/A | TTGTTTTTTTCCTCTTCATT | 41 | F | 422 |
| 1549624 | 40903 | 40922 | N/A | N/A | GCATCTGTTTTTTAATGATT | 55 | F | 423 |
| 1549630 | 60001 | 60020 | N/A | N/A | TTGTATATTTCTAATATCAC | 89 | F | 424 |
| 1549642 | 34366 | 34385 | N/A | N/A | CTGTCACTTTTCCATACTAA | 36 | F | 425 |
| 1549646 | 118460 | 118479 | 9586 | 9605 | AGCTGCTTTATCATCTTCAT | 103 | F | 426 |
| 1549661 | 39475 | 39494 | N/A | N/A | GCATTTTTTCCTATAATCTT | 16 | F | 427 |
| 1549666 | 114003 | 114022 | 5129 | 5148 | CTTTCTCTTCTTCCTGGAGA | 78 | F | 428 |
| 1549671 | 114341 | 114360 | 5467 | 5486 | GCTTTTTTAAATGTAATCCT | 64 | F | 429 |
| 1549673 | 32841 | 32860 | N/A | N/A | CGCACATAATTCTTCATCTT | 54 | F | 430 |
| 1549678 | 115853 | 115872 | 6979 | 6998 | TGTTCTCCCCTACCTAACCC | 72 | F | 431 |
| 1549681 | 52714 | 52733 | N/A | N/A | ACTATTTTCATCACATCACT | 77 | F | 432 |
| 1549685 | 47269 | 47288 | N/A | N/A | ACTCTTGATTTCCTAATCTA | 110 | F | 433 |
| 1549697 | 70250 | 70269 | N/A | N/A | TCTCATTATTGATTATGCCA | 13 | F | 434 |
| 1549698 | 7468 | 7487 | 4010 | 4029 | CATTCTTACTGATTTTTTTC | 52 | F | 435 |
| 1549701 | 72415 | 72434 | N/A | N/A | CTGGATATACTAACCACCCC | 89 | F | 436 |
| 1549702 | 21806 | 21825 | N/A | N/A | GCATATGTATATATCATAAT | 52 | F | 437 |
| 1549703 | 95896 | 95915 | N/A | N/A | TTGCCTTTTTTTCTTCCCCA | 56 | F | 438 |
| 1549709 | 12334 | 12353 | N/A | N/A | CTACTATCTTTCTTCTCCAT | 58 | F | 439 |
| 1549712 | 4748 | 4767 | N/A | N/A | ACTGTGTTTCTTCTTTTCTC | 12 | F | 440 |
| 1549713 | 80007 | 80026 | N/A | N/A | CCTCTTTTTCCTCTTTTCCA | 25 | F | 441 |
| 1549715 | 68235 | 68254 | 4388 | 4407 | CCTCATGTCCACTATCCTTC | 86 | F | 442 |
| 1549718 | 97754 | 97773 | N/A | N/A | TCTTTTTCCATTTGTCCGCT | 24 | F | 443 |
| 1549726 | 94071 | 94090 | N/A | N/A | CCTAGCATTTCTCTTTTTCA | 61 | F | 444 |
| 1549731 | 115315 | 115334 | 6441 | 6460 | TTCACATTTTTAATCAAAAA | 137 | F | 445 |
| 1549748 | 18031 | 18050 | N/A | N/A | GCTTTCTTTCCATTTCAGAA | 36 | F | 446 |
| 1549751 | 43670 | 43689 | N/A | N/A | GCTGCTGTTTCTATTTTTAT | 40 | F | 447 |
| 1549757 | 116938 | 116957 | 8064 | 8083 | GTCACACTTCAACACCAACT | 40 | F | 448 |
| 1549761 | 29227 | 29246 | N/A | N/A | TTTGATCTATTTCTGACCTT | 69 | F | 449 |
| 1549769 | 82506 | 82525 | N/A | N/A | TCTGATTTTGATCCTCACTA | 72 | F | 450 |
| 1549780 | 54744 | 54763 | N/A | N/A | GCTTTTACTCTACTGTGTTC | 88 | F | 451 |
| 1549791 | 25732 | 25751 | N/A | N/A | ATTCTCTTTTTCCCTCCCTT | 35 | F | 452 |
| 1549793 | 117937 | 117956 | 9063 | 9082 | TCATCAGTTTTCCCTAATGA | 66 | F | 453 |
| 1549797 | 63665 | 63684 | N/A | N/A | GCTAAGATTTTCTTCCATTT | 15 | F | 454 |
| 1549798 | 18970 | 18989 | N/A | N/A | CGTGCATCTCCTATTCATCC | 11 | F | 455 |
| 1549801 | 24152 | 24171 | N/A | N/A | TTGTGTATCTATATCTCCCT | 30 | F | 456 |
| 1549802 | 12283 | 12302 | N/A | N/A | CTTCACTCTTTTCTACGCCC | 28 | F | 457 |
| 1549836 | 30256 | 30275 | N/A | N/A | GCTTCTTTCTTTACTTTGAA | 13 | F | 458 |
| 1549842 | 15629 | 15648 | N/A | N/A | GCTGTCATATTTTATTTCAT | 11 | F | 459 |
| 1549847 | 52788 | 52807 | N/A | N/A | TGTGTCATTTTTTTCTCACC | 70 | F | 460 |
| 1549850 | 107289 | 107308 | N/A | N/A | ACTATTTTATCATAAATACT | 134 | F | 461 |
| 1549854 | 114132 | 114151 | 5258 | 5277 | GTCACAGAATGATAATCACC | 67 | F | 462 |
| 1549857 | 82572 | 82591 | N/A | N/A | TATCTTATTTCATTCTCTCT | 110 | F | 463 |
| 1549858 | 4112 | 4131 | N/A | N/A | CTCTCTGTTTTTCCTACCAT | 26 | F | 464 |
| 1549869 | 59764 | 59783 | N/A | N/A | ATGCTTTTCTCAATTTTCCA | 44 | F | 465 |
| 1549875 | 10888 | 10907 | N/A | N/A | ATTAGCATTTATTACTCTAT | 17 | F | 466 |
| 1549877 | 38626 | 38645 | N/A | N/A | GCCTTATTTTTCTCCATATC | 56 | F | 467 |
| 1549890 | 25763 | 25782 | N/A | N/A | TTTCATGTATATATAAACCA | 77 | F | 468 |
| 1549893 | 13012 | 13031 | N/A | N/A | TTTATTTTCATTATTTCCAC | 63 | F | 469 |
| 1549898 | 6719 | 6738 | 3933 | 3952 | AGGCTCACTTTCTCCTCTGT | 36 | F | 470 |
| 1549904 | 20420 | 20439 | N/A | N/A | CTGGTATTTTTCTTTGTGCT | 8 | F | 471 |
| 1549905 | 85957 | 85976 | N/A | N/A | CTTGTTTGATTTTCATCCTA | 67 | F | 472 |
| 1549906 | 118341 | 118360 | 9467 | 9486 | TTTATCTTCATATAATCAGC | 41 | F | 473 |
| 1549917 | 110199 | 110218 | N/A | N/A | GCCACTTTATATATGAAATA | 53 | F | 474 |
| 1549920 | 17751 | 17770 | N/A | N/A | CCACTGTGTTCTCTCCCCTA | 32 | F | 475 |
| 1549921 | 55257 | 55276 | N/A | N/A | AGTCACTTTCTTATTATTTA | 60 | F | 476 |
| 1549949 | 73144 | 73163 | N/A | N/A | TCAATCACTCTTTTTAGAGA | 63 | F | 477 |
| | 75556 | 75575 | | | | | | |
| 1549953 | 73315 | 73334 | N/A | N/A | GGGATATCCACATCATCAAA | 98 | F | 478 |
| | 75727 | 75746 | | | | | | |
| 1549968 | 73217 | 73236 | N/A | N/A | AATTCAAGCCCCATGATACC | 120 | F | 479 |
| | 75629 | 75648 | | | | | | |
| 1549971 | 73209 | 73228 | N/A | N/A | CCCCATGATACCCCTAATCA | 114 | F | 480 |
| | 75621 | 75640 | | | | | | |
| 1549986 | 73362 | 73381 | N/A | N/A | ATAAACCCCAATCTCATATC | 111 | F | 481 |
| | 75774 | 75793 | | | | | | |
| 1549998 | 73347 | 73366 | N/A | N/A | ATATCAGGATTACATATTTA | 160 | F | 482 |
| | 75759 | 75778 | | | | | | |

### Example 2: Effect of Modified Oligonucleotides on Human PCDH19 In Vitro, Multiple Doses

SHSY5Y cells, plated at a density of 10,000 cells/well, were differentiated in Neurobasal media supplemented with B27 (ThermoFisher), penicillin/streptomycin (ThermoFisher), and 10 μM retinoic acid (Sigma) for 10 days. Differentiated SH-SY5Y cells were treated with modified oligonucleotides at concentrations indicated in the tables below by free uptake. After a treatment period of approximately 5 days, total RNA was isolated from the cells and PCDH19 RNA levels were measured by quantitative real-time RT-PCR using human PCDH19 primer-probe set RTS53390 (described herein above) was used to measure RNA levels. PCDH19 RNA levels were normalized to total RNA content, as measured by GAPDH. Human GAPDH was measured using the human primer-probe set RTS104 (forward sequence GAAGGTGAAGGTCGGAGTC, designated herein as SEQ ID NO: 6; reverse sequence GAAGATGGTGATGGGATTTC, designated herein as SEQ ID NO: 7; probe sequence CAAGCTTCCCGTTCTCAGCC, designated herein as SEQ ID NO: 8). Reduction of PCDH19 RNA is presented in the tables below as percent PCDH19 RNA, relative to untreated control cells (% UTC). “N.C.” refers to values that were not calculated.

The half maximal inhibitory concentration (IC₅₀) of each modified oligonucleotide was calculated using a linear regression on a log/linear plot of the data in Excel and is also presented in the table below.

TABLE 2
Dose-dependent reduction of human PCDH19 RNA in differentiated
SH-SY5Y cells by modified oligonucleotides

| | PCDH19 RNA (% UTC) RTS53390 | | | | |
|---|---|---|---|---|---|
| Compound No. | 240 nM | 1200 nM | 6000 nM | 30000 nM | IC₅₀ (μM) |
| 1549513 | 41 | 12 | 6 | 3 | <0.24 |
| 1549558 | 42 | 16 | 9 | 1 | <0.24 |
| 1549562 | 64 | 34 | 13 | 5 | 0.50 |
| 1549589 | 23 | 12 | 4 | 4 | <0.24 |
| 1549605 | 68 | 54 | 32 | 27 | 1.44 |
| 1549626 | 75 | 70 | 60 | 19 | 4.73 |
| 1549697 | 37 | 16 | 7 | 5 | <0.24 |
| 1549712 | 38 | 22 | 11 | 5 | <0.24 |
| 1549784 | 22 | 7 | 4 | 3 | <0.24 |
| 1549798 | 35 | 21 | 6 | 5 | <0.24 |
| 1549841 | 59 | 31 | 17 | 5 | 0.36 |
| 1549842 | 33 | 11 | 5 | 3 | <0.24 |
| 1549855 | 23 | 8 | 5 | 5 | <0.24 |
| 1549887 | 69 | 32 | 10 | 5 | 0.56 |
| 1549904 | 73 | 28 | 13 | 5 | 0.60 |
| 1549911 | 47 | 26 | 16 | 4 | <0.24 |
| 1549915 | 57 | 31 | 39 | 13 | 0.36 |
| 1549938 | 23 | 9 | 4 | 3 | <0.24 |
| 1549980 | 81 | 52 | 29 | 10 | 1.65 |

TABLE 3
Dose-dependent reduction of human PCDH19 RNA in differentiated
SH-SY5Y cells by modified oligonucleotides

| | PCDH19 RNA (% UTC) RTS53390 | | | | |
|---|---|---|---|---|---|
| Compound No. | 240 nM | 1200 nM | 6000 nM | 30000 nM | IC₅₀ (μM) |
| 1549517 | 35 | 13 | 11 | 4 | <0.24 |
| 1549518 | 28 | 14 | 8 | 5 | <0.24 |
| 1549519 | 42 | 19 | 9 | 6 | <0.24 |
| 1549520 | 56 | 39 | 25 | 6 | 0.44 |
| 1549526 | 43 | 22 | 7 | 8 | <0.24 |
| 1549534 | 35 | 15 | 6 | 2 | <0.24 |
| 1549566 | 26 | 15 | 4 | 2 | <0.24 |
| 1549572 | 38 | 15 | 6 | 3 | <0.24 |
| 1549613 | 52 | 25 | 10 | N.C | 0.24 |
| 1549628 | 23 | 9 | 4 | 5 | <0.24 |
| 1549654 | 69 | 35 | 19 | 7 | 0.67 |
| 1549700 | 50 | 33 | 27 | N.C | <0.24 |
| 1549737 | 62 | 36 | 20 | 7 | 0.51 |
| 1549739 | 32 | 11 | 6 | 4 | <0.24 |
| 1549800 | 32 | 13 | 7 | 4 | <0.24 |
| 1549804 | 51 | 31 | 14 | 3 | 0.22 |
| 1549866 | 33 | 8 | 7 | 4 | <0.24 |
| 1549887 | 45 | 19 | 9 | 9 | <0.24 |
| 1549941 | 33 | 14 | 8 | 7 | <0.24 |

### Example 3: Effect of 3-10-3 cEt Modified Oligonucleotides with Uniform Phosphorothioate Internucleoside Linkages Complementary to a Human PCDH19 RNA In Vitro, Single Dose

Modified oligonucleotides complementary to human PCDH19 nucleic acid were designed and tested for their single dose effects on PCDH19 RNA in vitro. The modified oligonucleotides were tested in a series of experiments that had the same culture conditions.

The modified oligonucleotides in the table below are 3-10-3 cEt modified oligonucleotides with uniform phosphorothioate internucleoside linkages. The modified oligonucleotides are 16 nucleosides in length. The sugar motif for the modified oligonucleotides is (from 5′ to 3′): kkkddddddddddkkk; wherein each “d” represents a 2′-β-D-deoxyribosyl sugar moiety, and each “k” represents a cEt sugar moiety. The internucleoside linkage motif for the modified oligonucleotides is (from 5′ to 3′): sssssssssssssss; wherein each “s” represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 1 (described herein above), to SEQ ID NO: 2 (described herein above), or to both. “N/A” indicates that the modified oligonucleotide is not 100% complementary to that particular target nucleic acid sequence.

SHSY5Y cells, plated at a density of 10,000 cells/well, were differentiated in Neurobasal media supplemented with B27 (ThermoFisher), penicillin/streptomycin (ThermoFisher), and 10 μM retinoic acid (Sigma) for 10 days. Differentiated SH-SY5Y cells were treated with modified oligonucleotide at a concentration of 6,000 nM by free uptake. After a treatment period of approximately 5 days, total RNA was isolated from the cells and PCDH19 RNA levels were measured by quantitative real-time RT-PCR using human primer-probe set RTS53390 (described herein above). PCDH19 RNA levels were normalized to total RNA content, as measured by RIBOGREEN®. Reduction of PCDH19 RNA is presented in the table below as percent PCDH19 RNA relative to the amount in untreated control cells (% UTC).

Each separate experiment described in this example is identified by an Assay Identification letter in the table column labeled “AID”.

TABLE 4
Reduction of PCDH19 RNA by 3-10-3 cEt modified oligonucleotides with uniform phosphorothioate
internucleoside linkages at a concentration of 6,000 nM in differentiated SH-SY5Y cells

| | SEQ ID | SEQ ID | SEQ ID | SEQ ID | | | | |
|---|---|---|---|---|---|---|---|---|
| | NO: 1 | NO: 1 | NO: 2 | NO: 2 | | | | SEQ |
| Compound | Start | Stop | Start | Stop | | PCDH19 | | ID |
| No. | Site | Site | Site | Site | Sequence (5′ to 3′) | (% UTC) | AID | NO |
| 1626338 | 31494 | 31509 | N/A | N/A | GACTATACTAAGCTGC | 46 | G | 483 |
| 1626339 | 80695 | 80710 | N/A | N/A | AGATATTTACACGCAG | 11 | G | 484 |
| 1626340 | 82939 | 82954 | N/A | N/A | CGAACAAATAAGGTCA | 30 | G | 485 |
| 1626341 | 61843 | 61858 | N/A | N/A | TAGGTATTATGCTTCA | 18 | G | 486 |
| 1626342 | 38804 | 38819 | N/A | N/A | GGATATAAAGGTAGGT | 7 | G | 487 |
| 1626343 | 108445 | 108460 | N/A | N/A | ATAACTAATTGCCTGC | 77 | G | 488 |
| 1626344 | 17864 | 17879 | N/A | N/A | TAGTATTTGAGATTCG | 3 | G | 489 |
| 1626345 | 21721 | 21736 | N/A | N/A | AGATTAACAACCCTGT | 37 | G | 490 |
| 1626346 | 38972 | 38987 | N/A | N/A | CCATATGTAGTATGTG | 70 | G | 491 |
| 1626347 | 31852 | 31867 | N/A | N/A | GTCAATACGGTTCTTT | 18 | G | 492 |
| 1626348 | 96905 | 96920 | N/A | N/A | GAATTTGAAGCTCCGT | 24 | G | 493 |
| 1626349 | 100640 | 100655 | N/A | N/A | CTGAATAAGGGTATCC | 93 | G | 494 |
| 1626350 | 73501 | 73516 | N/A | N/A | GAGCAATTAGAGTCAT | 24 | G | 495 |
| 1626351 | 107870 | 107885 | N/A | N/A | GACTAAGGTAGATCTC | 70 | G | 496 |
| 1626352 | 109959 | 109974 | N/A | N/A | ATTGTATAGGACTTGT | 65 | G | 497 |
| 1626353 | 72416 | 72431 | N/A | N/A | GATATACTAACCACCC | 51 | G | 498 |
| 1626354 | 100639 | 100654 | N/A | N/A | TGAATAAGGGTATCCA | 65 | G | 499 |
| 1626355 | 9559 | 9574 | N/A | N/A | GCAGAATACCATCAGT | 16 | G | 500 |
| 1626356 | 47246 | 47261 | N/A | N/A | GAAATAATATCAGCCG | 49 | G | 501 |
| 1626357 | 29081 | 29096 | N/A | N/A | TCTTACTAGTAGGTGA | 47 | G | 502 |
| | 63965 | 63980 | | | | | | |
| 1626358 | 21701 | 21716 | N/A | N/A | GAATTTTTACGCTGAT | 17 | G | 503 |
| 1626359 | 94399 | 94414 | N/A | N/A | ACTAATAGGCATCCCA | 39 | G | 504 |
| 1626360 | 116339 | 116354 | 7465 | 7480 | GACAATTAATGTTCCC | 33 | G | 505 |
| 1626361 | 117452 | 117467 | 8578 | 8593 | GTCAATAAGGGTCTTA | 13 | G | 506 |
| 1626362 | 86726 | 86741 | N/A | N/A | GTAGATAAGAACTGCC | 70 | G | 507 |
| 1626363 | 64064 | 64079 | N/A | N/A | GACGAATGAACATCAC | 66 | G | 508 |
| 1626364 | 32404 | 32419 | N/A | N/A | CCAAATAGGGTTCGAT | 30 | G | 509 |
| 1626365 | 106683 | 106698 | N/A | N/A | ATCCGTAAAACATTCA | 39 | G | 510 |
| 1626366 | 98758 | 98773 | N/A | N/A | TACTATACATGAGTCC | 39 | G | 511 |
| 1626367 | 18811 | 18826 | N/A | N/A | GAATATGCTGCCCTCA | 63 | G | 512 |
| 1626368 | 97739 | 97754 | N/A | N/A | TTAATTATACTAGTCG | 75 | G | 513 |
| 1626369 | 93924 | 93939 | N/A | N/A | GCATTACAATCCCCAG | 25 | G | 514 |
| 1626370 | 115090 | 115105 | 6216 | 6231 | CTAATCATGATTGGGA | 30 | G | 515 |
| 1626371 | 50677 | 50692 | N/A | N/A | CCCATATAAGATCTCC | 38 | G | 516 |
| 1626372 | 29082 | 29097 | N/A | N/A | ATCTTACTAGTAGGTG | 65 | G | 517 |
| | 63966 | 63981 | | | | | | |
| 1626373 | 98771 | 98786 | N/A | N/A | GTAGAATAGGCCATAC | 86 | G | 518 |
| 1626374 | 60537 | 60552 | N/A | N/A | GCAGAATAACCACTGG | 67 | G | 519 |
| 1626375 | 73355 | 73370 | N/A | N/A | TCTCATATCAGGATTA | 16 | G | 520 |
| | 75767 | 75782 | | | | | | |
| 1626376 | 116212 | 116227 | 7338 | 7353 | CATAATATCAGGATGG | 28 | G | 521 |
| 1626377 | 73354 | 73369 | N/A | N/A | CTCATATCAGGATTAC | 5 | G | 522 |
| | 75766 | 75781 | | | | | | |
| 1626378 | 29479 | 29494 | N/A | N/A | CCAAATCAAAACCCGT | 25 | G | 523 |
| 1626379 | 35977 | 35992 | N/A | N/A | GATAATTACCAGTCTA | 65 | G | 524 |
| 1626380 | 92752 | 92767 | N/A | N/A | CCAATAAAAGTTCGGT | 75 | G | 525 |
| 1626381 | 18505 | 18520 | N/A | N/A | CTAGTAATAACAGGCT | 67 | G | 526 |
| 1626382 | 67011 | 67026 | N/A | N/A | GATTATGAGATTCCTC | 62 | G | 527 |
| 1626383 | 82952 | 82967 | N/A | N/A | AAAATTGAGGATTCGA | 43 | G | 528 |
| 1626384 | 20228 | 20243 | N/A | N/A | ACCAATTACATGGCAC | 28 | G | 529 |
| 1626385 | 4425 | 4440 | N/A | N/A | CACTTTATATAGCCAG | 10 | G | 530 |
| 1626386 | 18927 | 18942 | N/A | N/A | AAGGTTAGAATACCCA | 101 | G | 531 |
| 1626387 | 57855 | 57870 | N/A | N/A | CATCTACGATATTCAG | 37 | G | 532 |
| 1626388 | 73500 | 73515 | N/A | N/A | AGCAATTAGAGTCATC | 7 | G | 533 |
| 1626389 | 94401 | 94416 | N/A | N/A | GCACTAATAGGCATCC | 78 | G | 534 |
| 1626390 | 93953 | 93968 | N/A | N/A | GAATATCATTGATTGG | 23 | G | 535 |
| 1626391 | 68433 | 68448 | N/A | N/A | AAGTAGTATAGTGTGC | 31 | G | 536 |
| 1626392 | 9470 | 9485 | N/A | N/A | ATCAATGTAAGTGGTC | 36 | G | 537 |
| 1626393 | 84590 | 84605 | N/A | N/A | GGACTATAATGATCTT | 76 | G | 538 |
| | 84903 | 84918 | | | | | | |
| 1626394 | 98651 | 98666 | N/A | N/A | GCATTAGTAAATCTCC | 47 | G | 539 |
| 1626395 | 23090 | 23105 | N/A | N/A | GTTTATACAGCCATCA | 28 | G | 540 |
| 1626396 | 28923 | 28938 | N/A | N/A | CTATAGTATATGTCTG | 65 | G | 541 |
| 1626397 | 107970 | 107985 | N/A | N/A | CTTTATTACTAGCGGT | 47 | G | 542 |
| 1626398 | 6356 | 6371 | N/A | N/A | GGAAATTCGATTTCTG | 61 | G | 543 |
| 1626399 | 94944 | 94959 | N/A | N/A | GAAAATTTCGAGTCTT | 55 | G | 544 |
| 1626400 | 28468 | 28483 | N/A | N/A | GATGTAATATGGGCAA | 5 | G | 545 |
| 1626401 | 97740 | 97755 | N/A | N/A | CTTAATTATACTAGTC | 76 | G | 546 |
| 1626402 | 50676 | 50691 | N/A | N/A | CCATATAAGATCTCCC | 84 | G | 547 |
| 1626403 | 114384 | 114399 | 5510 | 5525 | CTTATTAAAGGTGTCC | 29 | G | 548 |
| 1626404 | 73836 | 73851 | N/A | N/A | AAGCATAGTACGCAGC | 32 | G | 549 |
| 1626405 | 68430 | 68445 | N/A | N/A | TAGTATAGTGTGCCTA | 47 | G | 550 |
| 1626406 | 68165 | 68180 | N/A | N/A | GATAATTTACGACCGT | 77 | G | 551 |
| 1626407 | 9325 | 9340 | N/A | N/A | GCAGTATAGAGTCCTT | 6 | G | 552 |
| 1626408 | 69088 | 69103 | N/A | N/A | CTCGATAAAGCATGTG | 18 | G | 553 |
| 1626409 | 26542 | 26557 | N/A | N/A | GACTTAACATGACTCC | 48 | G | 554 |
| 1626410 | 11934 | 11949 | N/A | N/A | CTTGATTAGTGTTCCA | 8 | G | 555 |
| 1626411 | 68289 | 68304 | 4442 | 4457 | ACAATACAGGCTCCGC | 51 | G | 556 |
| 1626412 | 61423 | 61438 | N/A | N/A | TAAGATGCAAGGGTCC | 29 | G | 557 |
| 1626413 | 93017 | 93032 | N/A | N/A | GATACTTAAGTTCTGC | 18 | G | 558 |
| 1626414 | 50392 | 50407 | N/A | N/A | ACAGTATGAGCTCTCC | 74 | G | 559 |
| 1626415 | 63951 | 63966 | N/A | N/A | GATACTACATACCCAG | 43 | G | 560 |

### Example 4: Effect of Modified Oligonucleotides on Human PCDH19 In Vitro, Multiple Doses

SHSY5Y cells, plated at a density of 8,000 cells/well, were differentiated in Neurobasal media supplemented with B27 (ThermoFisher), penicillin/streptomycin (ThermoFisher), and 10 μM retinoic acid (Sigma) for 10 days. Differentiated SH-SY5Y cells were treated with modified oligonucleotides at concentrations indicated in the table below by free uptake. After a treatment period of approximately 5 days, total RNA was isolated from the cells and PCDH19 RNA levels were measured by quantitative real-time RT-PCR using human PCDH19 primer-probe set RTS53390 (described herein above) was used to measure RNA levels. PCDH19 RNA levels were normalized to total RNA content, as measured by GAPDH. Human GAPDH was measured using the human primer-probe set RTS104 (described herein above). Reduction of PCDH19 RNA is presented in the tables below as percent PCDH19 RNA, relative to untreated control cells (% UTC).

The half maximal inhibitory concentration (IC₅₀) of each modified oligonucleotide was calculated using a linear regression on a log/linear plot of the data in Excel and is also presented in the table below.

TABLE 5
Dose-dependent reduction of human PCDH19 RNA in differentiated
SH-SY5Y cells by modified oligonucleotides

| | PCDH19 RNA (% UTC) RTS53390 | | | | |
|---|---|---|---|---|---|
| Compound No. | 46 nM | 278 nM | 1667 nM | 10000 nM | IC₅₀ (μM) |
| 1626339 | 47 | 16 | 8 | 2 | <0.046 |
| 1626341 | 135 | 71 | 20 | 1 | 0.88 |
| 1626342 | 91 | 19 | 6 | 3 | 0.19 |
| 1626344 | 16 | 5 | 1 | 0.4 | <0.046 |
| 1626347 | 134 | 47 | 26 | 5 | 0.78 |
| 1626348 | 128 | 58 | 29 | 15 | 1.00 |
| 1626355 | 72 | 41 | 17 | 2 | 0.18 |
| 1626358 | 119 | 54 | 12 | 4 | 0.54 |
| 1626361 | 18 | 13 | 7 | 7 | <0.046 |
| 1626375 | 125 | 52 | 17 | 4 | 0.66 |
| 1626377 | 16 | 6 | 2 | 0.2 | <0.046 |
| 1626383 | 152 | 72 | 42 | 19 | 1.64 |
| 1626385 | 48 | 21 | 7 | 1 | <0.046 |
| 1626388 | 48 | 27 | 8 | 5 | <0.046 |
| 1626395 | 133 | 45 | 19 | 3 | 0.68 |
| 1626400 | 49 | 9 | 2 | 0.1 | <0.046 |
| 1626407 | 15 | 2 | 0.2 | 0 | <0.046 |
| 1626408 | 132 | 69 | 30 | 14 | 1.13 |
| 1626410 | 70 | 5 | 2 | 1 | 0.05 |

### Example 5: Design of 5-10-5 MOE Gapmer Modified Oligonucleotides with PS Internucleoside Linkages that Target a Human PCDH19 Nucleic Acid

Modified oligonucleotides complementary to a human PCDH19 nucleic acid were designed, as described in the table below. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the table below is 100% complementary to SEQ ID NO: 1 (described herein above), to SEQ ID NO: 2 (described herein above), or to both. ‘N/A’ indicates that the modified oligonucleotide has two or more mismatches to that particular target nucleic acid sequence.

The modified oligonucleotides in the table below are 5-10-5 MOE modified oligonucleotides with uniform phosphorothioate internucleoside linkages. The modified oligonucleotides are 20 nucleosides in length, wherein the central gap segment consists of ten 2′-β-D-deoxynucleosides, and wherein the 5′ and 3′ wing segments each consist of five 2′-MOE nucleosides. The sugar motif for the modified oligonucleotides is (from 5′ to 3′): eeeeeddddddddddeeeee; wherein each “d” represents a 2′-β-D-deoxyribosyl sugar moiety, and each “e” represents a 2′-MOE ribosyl sugar moiety. The internucleoside linkage motifs for the modified oligonucleotides is (from 5′ to 3′): sssssssssssssssssss; wherein each “s” represents a phosphorothioate internucleoside linkage. All cytosine nucleobases are 5-methylcytosines.

TABLE 6
5-10-5 MOE modified oligonucleotides with uniform phosphorothioate internucleoside linkages
complementary to human PCDH19

| | SEQ ID | SEQ ID | SEQ ID | SEQ ID | | SEQ |
|---|---|---|---|---|---|---|
| Compound | NO: 1 | NO: 1 | NO: 2 | NO: 2 | | ID |
| No. | Start Site | Stop Site | Start Site | Stop Site | Sequence (5′ to 3′) | NO |
| 1653805 | 10648 | 10667 | N/A | N/A | GTGTCATTTTCCCTACGCAG | 75 |
| 1653806 | 10817 | 10836 | N/A | N/A | CTTGTCTTTTTCATCTCTCT | 241 |
| 1653807 | 73316 | 73335 | N/A | N/A | TGGGATATCCACATCATCAA | 90 |
| | 75728 | 75747 | | | | |
| 1653808 | 32756 | 32775 | N/A | N/A | GCACTCATTTTTGTAAACTC | 341 |
| 1653809 | 43411 | 43430 | N/A | N/A | GCATTTCTTTTCTACCAGTA | 171 |
| 1653810 | 115316 | 115335 | 6442 | 6461 | GTTCACATTTTTAATCAAAA | 82 |
| 1653811 | 17984 | 18003 | N/A | N/A | GCACACTTCTTTTTCTATTT | 267 |
| 1653812 | 10818 | 10837 | N/A | N/A | TCTTGTCTTTTTCATCTCTC | 320 |
| 1653813 | 7441 | 7460 | 3983 | 4002 | TTGATTTCTTTTGATGCCCA | 124 |
| 1653814 | 41756 | 41775 | N/A | N/A | ATGCTTGTTTTCTTCCTCTC | 143 |
| 1653815 | 5510 | 5529 | N/A | N/A | GCTGATTTCTTTTTCATCAA | 251 |
| 1653816 | 21748 | 21767 | N/A | N/A | TTCCGATGTTTTATCCTCAT | 369 |
| 1653817 | 4534 | 4553 | N/A | N/A | GCTATTTTTACTGAAACATC | 63 |
| 1653818 | 12550 | 12569 | N/A | N/A | AGTTGCATTTTCTTCCTCCA | 333 |
| 1653819 | 20420 | 20439 | N/A | N/A | CTGGTATTTTTCTTTGTGCT | 471 |
| 1653820 | 80720 | 80739 | N/A | N/A | GCATCAGTTTTTCTTCTAAA | 216 |
| 1653821 | 34367 | 34386 | N/A | N/A | GCTGTCACTTTTCCATACTA | 20 |
| 1653822 | 78891 | 78910 | N/A | N/A | GTCTGTTTTATCTATCCACT | 329 |
| 1653823 | 25522 | 25541 | N/A | N/A | ACATCATTTTTCATGTGGTC | 21 |
| 1653824 | 43531 | 43550 | N/A | N/A | GCACATCATTCTTATTTATT | 379 |
| 1653825 | 10821 | 10840 | N/A | N/A | ATTTCTTGTCTTTTTCATCT | 391 |
| 1653826 | 72604 | 72623 | N/A | N/A | ATTCTCTCTCATTTCCACCC | 22 |
| 1653827 | 59851 | 59870 | N/A | N/A | CGTACATTTTCATACCAAGA | 95 |
| 1653828 | 17980 | 17999 | N/A | N/A | ACTTCTTTTTCTATTTCTAA | 94 |

### Example 6: Design of RNAi Compounds that Target a Human PCDH19 Nucleic Acid

RNAi compounds comprising antisense RNAi oligonucleotides complementary to a human PCDH19 nucleic acid, and sense RNAi oligonucleotides complementary to the antisense RNAi oligonucleotides were designed as follows.

The antisense RNAi oligonucleotide in each case is 23 nucleosides in length; has a sugar motif (from 5′ to 3′) of: yfyyyfyyyyyyyfyfyyyyyyy; wherein each ‘y’ represents a 2′-O-methylribosyl sugar moiety and each “f” represents a 2′-fluororibosyl sugar; and an internucleoside linkage motif (from 5′ to 3′) of: ssooooooooooooooooooss; wherein ‘o’ represents a phosphodiester internucleoside linkage and ‘s’ represents a phosphorothioate internucleoside linkage. Each cytosine residue is a non-methylated cytosine. Each antisense RNAi oligonucleotide has a terminal phosphate at the 5′-end. The antisense RNAi oligonucleotides are listed below in Tables 7 and 8.

“Start site” indicates the 5′-most nucleoside to which the antisense RNAi oligonucleotide is complementary in the target nucleic acid sequence. “Stop site” indicates the 3′-most nucleoside to which the antisense RNAi oligonucleotide is complementary in the target nucleic acid sequence. Each antisense RNAi oligonucleoside listed in the Table 7 below is 100% complementary to SEQ ID NO: 1 (ENSEMBL Accession No. ENSG00000165194.15 from version 104: May 2021), to SEQ ID NO: 2 (ENSEMBL Accession No. ENST00000373034.8 from version 104: May 2021), or to both. ‘N/A’ indicates that the antisense RNAi oligonucleotide is not 100% complementary to that particular target nucleic acid sequence in Table 7 below.

TABLE 7
Design of antisense strand modified oligonucleotides targeted to human PCDH19

| | SEQ ID | SEQ ID | SEQ ID | SEQ ID | | |
|---|---|---|---|---|---|---|
| Antisense | NO: 1 | NO: 1 | NO: 2 | NO: 2 | | |
| Strand | Strand | Strand | Strand | Strand | | |
| Compound | Start Site | Stop Site | Start Site | Stop Site | | SEQ |
| No. | Antisense | Antisense | Antisense | Antisense | Antisense Strand Sequence (5′ to 3′) | ID NO |
| 1688321 | 327 | 349 | 327 | 349 | UGUCUCGCUUUCUCUGUCUGUCU | 561 |
| 1688324 | 329 | 351 | 329 | 351 | UCUGUCUCGCUUUCUCUGUCUGU | 562 |
| 1688327 | 408 | 430 | 408 | 430 | UUGAUUUCAUCUUCCCGGAGAGG | 563 |
| 1688339 | 697 | 719 | 697 | 719 | UCAAAGCCCCGGUCUUUCUAAUG | 564 |
| 1688357 | 785 | 807 | 785 | 807 | UUCGUCUAGGAAAUCAAAGUUAA | 565 |
| 1688363 | 1254 | 1276 | 1254 | 1276 | UACUUGAGGCGAAGGUAAAGAGG | 566 |
| 1688369 | 1359 | 1381 | 1359 | 1381 | UUUACCCCUUCAAAGUUAGCCGG | 567 |
| 1688381 | 1752 | 1774 | 1752 | 1774 | UGCUCCUCUUCUACCGAGUACUU | 568 |
| 1688399 | 1987 | 2009 | 1987 | 2009 | UUCCAUUGAGCUGGACAUGACCU | 569 |
| 1688417 | 2235 | 2257 | 2235 | 2257 | UUUUCCACCACGAGUUCGGCAAA | 570 |
| 1688423 | 2352 | 2374 | 2352 | 2374 | UCAUUGGAGUCGGUCACCUUGAU | 571 |
| 1688432 | 2493 | 2515 | 2493 | 2515 | UAGCCAUAGAAGGAGUAGACCAC | 572 |
| 1688435 | 2502 | 2524 | 2502 | 2524 | UCGUUGACGUAGCCAUAGAAGGA | 573 |
| 1688438 | 2571 | 2593 | 2571 | 2593 | UAGUCUAAAGCGCCAGUGACAGU | 574 |
| 1688441 | 2577 | 2599 | 2577 | 2599 | UCUUCGUAGUCUAAAGCGCCAGU | 575 |
| 1688444 | 2694 | 2716 | 2694 | 2716 | UUGAUGACCGGCGGAUUGUCAUU | 576 |
| 1688447 | 2709 | 2731 | 2709 | 2731 | UUGACUGACAGCAGGUUGAUGAC | 577 |
| 1688450 | 2724 | 2746 | 2724 | 2746 | UCCACAAGCUCACUGUUGACUGA | 578 |
| 1688459 | 2877 | 2899 | 2877 | 2899 | UCCACCAGAAUAGUGGAGAAGCU | 579 |
| 1688462 | 2920 | 2942 | 2920 | 2942 | UGUGAGGUUGUAUUGGUCGUGCU | 580 |
| 1688468 | 2984 | 3006 | 2984 | 3006 | UGAUGAGCACGGUAAAGGACUUG | 581 |
| 1688471 | 2999 | 3021 | 2999 | 3021 | UGUCAUUUUCGUCAGUGAUGAGC | 582 |
| 1688480 | 3030 | 3052 | 3030 | 3052 | UGGUAGUAGGGCUUGGAAAAGUG | 583 |
| 1688483 | 3045 | 3067 | 3045 | 3067 | UCCUGCACAAUGACCUGGUAGUA | 584 |
| 1688492 | 3186 | 3208 | 3186 | 3208 | UUGGGAUUGAUGGAGACAUAGGU | 585 |
| 1688495 | 3201 | 3223 | 3201 | 3223 | UAGAUGUCGCCUGAGUUGGGAUU | 586 |
| 1688498 | 3219 | 3241 | 3219 | 3241 | UUAAAGGAUCGCAGCGCGUAGAU | 587 |
| 1688501 | 3234 | 3256 | 3234 | 3256 | UUGGUCUGCUCGUGGUUAAAGGA | 588 |
| 1688504 | 3249 | 3271 | 3249 | 3271 | UUGAAUUCGAACGCCUUGGUCUG | 589 |
| 1688507 | 3261 | 3283 | 3261 | 3283 | UUGGCCAGCACCUUGAAUUCGAA | 590 |
| 1688510 | 3330 | 3352 | 3330 | 3352 | UUGUCGUUGACGUCGAGGAUGAU | 591 |
| 1688513 | 3363 | 3385 | 3363 | 3385 | UUAAUCAGAGGUGGGGCUGUGAU | 592 |
| 1688519 | 3438 | 3460 | 3438 | 3460 | UAGUCUUCUGCCUUGACAACAGU | 593 |
| 1688522 | 3453 | 3475 | 3453 | 3475 | UUUUCGCCCUCAUCGUAGUCUUC | 594 |
| 1688528 | 3507 | 3529 | 3507 | 3529 | UCUAUUUCAAAGAAGCCGCGGUC | 595 |
| 1688531 | 3522 | 3544 | 3522 | 3544 | UCGCCAUUGACCUGGUCUAUUUC | 596 |
| 1688537 | 3575 | 3597 | 3575 | 3597 | UAAGCUCAUAGGAGGACUUGGAG | 597 |
| 1688540 | 3588 | 3610 | 3588 | 3610 | UGAGCCACCACGAUAAGCUCAUA | 598 |
| 1688549 | 3681 | 3703 | 3681 | 3703 | UUCACAGAGCCCAUUGACUCUUG | 599 |
| 1688561 | 3737 | 3759 | 3737 | 3759 | UUACAAAGAGGAUGCCCGCAAUG | 600 |
| 1688576 | N/A | N/A | 3811 | 3833 | UAAACAAUUACUGCAGUUGUAGG | 601 |
| 1688582 | 6627 | 6649 | 3841 | 3863 | UAUAAAACAGCCGAGGAGACAAG | 602 |
| 1688594 | 6680 | 6702 | 3894 | 3916 | UCGCUAAUGGGAGAAACCGAGAU | 603 |
| 1688597 | 6695 | 6717 | 3909 | 3931 | UUUUUGUCUUGCUCCUCGCUAAU | 604 |
| 1688603 | 6725 | 6747 | 3939 | 3961 | UUUCCCCUUAGGCUCACUUUCUC | 605 |
| 1688606 | N/A | N/A | 3954 | 3976 | UACUCAGCAAUUCUCUUUCCCCU | 606 |
| 1688953 | 7568 | 7590 | 4110 | 4132 | UGCUGGUGGUAGUCAAAAUAGUU | 607 |
| 1688968 | 7649 | 7671 | 4191 | 4213 | UUAGCACUGGUGUUGCGGGUAUU | 608 |
| 1688971 | 7664 | 7686 | 4206 | 4228 | UGAUGGUAGAUGUGGUUAGCACU | 609 |
| 1688974 | 7679 | 7701 | 4221 | 4243 | UGGCUGUUGAAAGAGUGAUGGUA | 610 |
| 1688980 | 7709 | 7731 | 4251 | 4273 | UUGAUAAUCAGGUCAGGCUGCUG | 611 |
| 1688986 | N/A | N/A | 4281 | 4303 | UAGUUUUCAGUCUCAGGCAGAGG | 612 |
| 1688989 | 59573 | 59595 | 4296 | 4318 | UUGGAGUCAAAAGAAUAGUUUUC | 613 |
| 1688995 | 59602 | 59624 | 4325 | 4347 | UGAUUAAAUGGGCUCGGCUAUUC | 614 |
| 1689001 | 68200 | 68222 | 4353 | 4375 | UCUAAGUCCUUGAAGGUGGAGCU | 615 |
| 1689007 | 68227 | 68249 | 4380 | 4402 | UGUCCACUAUCCUUCAGGCUGUU | 616 |
| 1689016 | 68266 | 68288 | 4419 | 4441 | UGGACAUCAUGCUCACUGUCAGU | 617 |
| 1689028 | 68347 | 68369 | 4500 | 4522 | UGAUCAGGCAUCUGAGAUCCCAU | 618 |
| 1689034 | 113404 | 113426 | 4530 | 4552 | UCCCGGCAAUGAAAUCCUUCAUU | 619 |
| 1689043 | 113515 | 113537 | 4641 | 4663 | UCAAUAGACAGCGCGAUGAUGUU | 620 |
| 1689046 | 113530 | 113552 | 4656 | 4678 | UCAGCAGCAGUAGCUUCAAUAGA | 621 |
| 1689061 | 113602 | 113624 | 4728 | 4750 | UGGUCGCUGACAUCUUUCCCAAA | 622 |
| 1689076 | 113824 | 113846 | 4950 | 4972 | UUGACAUACUGCUCCAGAUCACG | 623 |
| 1689085 | 113892 | 113914 | 5018 | 5040 | UGACUUUCUCGCUAUCAGCUCCA | 624 |
| 1689091 | 113931 | 113953 | 5057 | 5079 | UGUUGCGACCUUCCUUCAGAAUG | 625 |
| 1689097 | 113950 | 113972 | 5076 | 5098 | UUCACACCAGGGGACUCUUUGUU | 626 |
| 1689106 | 113995 | 114017 | 5121 | 5143 | UCUUCUUCCUGGAGACUGGUUUA | 627 |
| 1689118 | 114054 | 114076 | 5180 | 5202 | UUGGUGAGCAAUUAAAACAAGAA | 628 |
| 1689124 | 114084 | 114106 | 5210 | 5232 | UCUGAAAUAUAGCCACUCAAGAA | 629 |
| 1689130 | 114114 | 114136 | 5240 | 5262 | UCACCUAUAAACAAUACAUUUAG | 630 |
| 1689136 | 114144 | 114166 | 5270 | 5292 | UUGAAACAAGGGACUGUCACAGA | 631 |
| 1689139 | 114150 | 114172 | 5276 | 5298 | UGCCUGUUGAAACAAGGGACUGU | 632 |
| 1689154 | 114228 | 114250 | 5354 | 5376 | UUGUCUCUGUUUCCCCAACAUCA | 633 |
| 1689157 | 114243 | 114265 | 5369 | 5391 | UUUUCACAACUGAAUUUGUCUCU | 634 |
| 1689166 | 114288 | 114310 | 5414 | 5436 | UUGACAUAGAAAAAUAUAUAAAU | 635 |
| 1689169 | 114302 | 114324 | 5428 | 5450 | UUUAUAUUAUAAUUUUGACAUAG | 636 |
| 1689175 | 114332 | 114354 | 5458 | 5480 | UUAAAUGUAAUCCUCCACAAACA | 637 |
| 1689178 | 114347 | 114369 | 5473 | 5495 | UCACUUUUUGCUUUUUUAAAUGU | 638 |
| 1689199 | 114452 | 114474 | 5578 | 5600 | UCACAUCACUGAAUCAAUGAAUA | 639 |
| 1689202 | 114467 | 114489 | 5593 | 5615 | UUUGGGGGCUCAAUUUCACAUCA | 640 |
| 1689208 | 114497 | 114519 | 5623 | 5645 | UGGGAAGUACUCGAGCUUCAGAA | 641 |
| 1689214 | 114534 | 114556 | 5660 | 5682 | UUUUAAAGCAGGGGUGUCCACAG | 642 |
| 1689235 | 114639 | 114661 | 5765 | 5787 | UUUUUAAGAAAAAGAAAACACCA | 643 |
| 1689238 | 114654 | 114676 | 5780 | 5802 | UGUAGUUUUAUUAUUUUUUUAAG | 644 |
| 1689244 | 114684 | 114706 | 5810 | 5832 | UGUCUCAGUUGUUUGCACUUUGU | 645 |
| 1689247 | 114699 | 114721 | 5825 | 5847 | UGCUCAUUCAUUUGCUGUCUCAG | 646 |
| 1689259 | 114759 | 114781 | 5885 | 5907 | UCACUACAUGAGUUAAAAAACGU | 647 |
| 1689280 | 114856 | 114878 | 5982 | 6004 | UAGCAGCCUAUCCAAAAAUGACU | 648 |
| 1689304 | 114970 | 114992 | 6096 | 6118 | UUCUAAGCCUGGAAACAUAACUU | 649 |
| 1689307 | 114985 | 115007 | 6111 | 6133 | UUGGAGAGAGGGAAUUUCUAAGC | 650 |
| 1689310 | 115000 | 115022 | 6126 | 6148 | UUCAAUUUUAGAUGAUUGGAGAG | 651 |
| 1689328 | 115090 | 115112 | 6216 | 6238 | UCUAAUUCUAAUCAUGAUUGGGA | 652 |
| 1689331 | 115105 | 115127 | 6231 | 6253 | UGACGUGCUUUGCAGUCUAAUUC | 653 |
| 1689346 | 115176 | 115198 | 6302 | 6324 | UACUGCUUACUCUAUCCCUACGU | 654 |
| 1689352 | 115206 | 115228 | 6332 | 6354 | UUACCUGUCUUUUCCAAAAUAUA | 655 |
| 1689361 | 115251 | 115273 | 6377 | 6399 | UCCGUUUAGAAGGCACAGUAAUU | 656 |
| 1689367 | 115281 | 115303 | 6407 | 6429 | UAAAAAGAUGAGUUCAAGUGUCU | 657 |
| 1689373 | 115311 | 115333 | 6437 | 6459 | UCACAUUUUUAAUCAAAAAACUA | 658 |
| 1689376 | 115326 | 115348 | 6452 | 6474 | UUAGUAUUUUCUUGUUCACAUUU | 659 |
| 1689385 | 115371 | 115393 | 6497 | 6519 | UAGCCUACAGCAAAAAGAGUUGG | 660 |
| 1689403 | 115461 | 115483 | 6587 | 6609 | UAAUAGACAAAAGAUAAAUGGGG | 661 |
| 1689406 | 115476 | 115498 | 6602 | 6624 | UUGGCUAGUGAAUAUUAAUAGAC | 662 |
| 1689409 | 115491 | 115513 | 6617 | 6639 | UGCAAAAAUACUAUGUUGGCUAG | 663 |
| 1689427 | 115581 | 115603 | 6707 | 6729 | UUGUUUCUCUAGAAACAACUCUC | 664 |
| 1689439 | 115641 | 115663 | 6767 | 6789 | UUCUCUCUGUGUAUUCCUGAAUU | 665 |
| 1689442 | 115656 | 115678 | 6782 | 6804 | UCUAACCUCUAGGCCUUCUCUCU | 666 |
| 1689445 | 115671 | 115693 | 6797 | 6819 | UUUGCAUUCUAGUGCUCUAACCU | 667 |
| 1689451 | 115701 | 115723 | 6827 | 6849 | UCUACCUAAACUUAAGAAAAUAA | 668 |
| 1689463 | 115761 | 115783 | 6887 | 6909 | UAAUGCGUAAAGCUAGGAACUCA | 669 |
| 1689469 | 115791 | 115813 | 6917 | 6939 | UCUCCCUCCUGUAAAAGAAAAUA | 670 |
| 1689472 | 115801 | 115823 | 6927 | 6949 | UCAAAACCCUUCUCCCUCCUGUA | 671 |
| 1689478 | 115868 | 115890 | 6994 | 7016 | UAAGUUCUUUUUCAGAAAUGUUC | 672 |
| 1689481 | 115883 | 115905 | 7009 | 7031 | UUUGUAGCUUCAUUAUAAGUUCU | 673 |
| 1689490 | 115928 | 115950 | 7054 | 7076 | UUUGCAUGAGGUAGUAUUCAGCA | 674 |
| 1689496 | 115958 | 115980 | 7084 | 7106 | UCAGAGGGAAAUUUGCAGCCACA | 675 |
| 1689499 | 115973 | 115995 | 7099 | 7121 | UUAAUUGUGUCAGCUUCAGAGGG | 676 |
| 1689502 | 115988 | 116010 | 7114 | 7136 | UAUGGUAAUUCAUCCUUAAUUGU | 677 |
| 1689517 | 116063 | 116085 | 7189 | 7211 | UCCACUACCAAAAUGAAAUAAAU | 678 |
| 1689523 | 116093 | 116115 | 7219 | 7241 | UGCAAAGAGAAAUAAAUACUUAA | 679 |
| 1689544 | 116198 | 116220 | 7324 | 7346 | UCAGGAUGGAUCAGGGUGAGAAA | 680 |
| 1689562 | 116288 | 116310 | 7414 | 7436 | UUCCCACUGAGCAUAGCAAGAAA | 681 |
| 1689607 | 116523 | 116545 | 7649 | 7671 | UGACUGCCCACUGAGAACAAAGU | 682 |
| 1689619 | 116582 | 116604 | 7708 | 7730 | UUCCUUAAUGAGAGGAAUUCUCU | 683 |
| 1689622 | 116597 | 116619 | 7723 | 7745 | UGUGGGGAAAAAUAUUUCCUUAA | 684 |
| 1689640 | 116678 | 116700 | 7804 | 7826 | UACAGCCUGCCUACAAAACCAGU | 685 |
| 1689691 | 116947 | 116969 | 8073 | 8095 | UCAUUAAAGCAUGUCACACUUCA | 686 |
| 1689694 | 116962 | 116984 | 8088 | 8110 | UUGCCAAAGCAGUUAUCAUUAAA | 687 |
| 1689697 | 116977 | 116999 | 8103 | 8125 | UACCUGUCUGGGGAAUUGCCAAA | 688 |
| 1689700 | 116992 | 117014 | 8118 | 8140 | UUGGAUGAAGAAGUAUACCUGUC | 689 |
| 1689706 | 117022 | 117044 | 8148 | 8170 | UUUGUCUAAAUGAACACCUUAGG | 690 |
| 1689712 | 117052 | 117074 | 8178 | 8200 | UAGAGAUCAGUGGAUCACUAAGC | 691 |
| 1689718 | 117077 | 117099 | 8203 | 8225 | UGUAUUUUUUAAAAAUAUUUCUG | 692 |
| 1689721 | 117092 | 117114 | 8218 | 8240 | UUCAGAGAUCUAAAUUGUAUUUU | 693 |
| 1689724 | 117107 | 117129 | 8233 | 8255 | UGAGUGGUUCCAAUUUUCAGAGA | 694 |
| 1689727 | 117122 | 117144 | 8248 | 8270 | UCAAGAUGAUACUCUUGAGUGGU | 695 |
| 1689742 | 117197 | 117219 | 8323 | 8345 | UAUGAUAGGGCUACAGGGAAAAG | 696 |
| 1689748 | 117227 | 117249 | 8353 | 8375 | UGCUUCAAGUGGACCACUUUUUG | 697 |
| 1689754 | 117252 | 117274 | 8378 | 8400 | UCCCUAAACUGCCCUUUGUCCUU | 698 |
| 1689757 | 117299 | 117321 | 8425 | 8447 | UUUUCCACUCUGGAUGCCCAAUC | 699 |
| 1689781 | 117419 | 117441 | 8545 | 8567 | UAAAGAGCCAAGUGGACCAAACU | 700 |
| 1689784 | 117434 | 117456 | 8560 | 8582 | UCUUAUUACCCGGGGUAAAGAGC | 701 |
| 1689790 | 117464 | 117486 | 8590 | 8612 | UUUAUGCCAGUUUUUCAAAGUCA | 702 |
| 1689808 | 117554 | 117576 | 8680 | 8702 | UACAAUUUGACUCAUGAAAUGAC | 703 |
| 1689811 | 117569 | 117591 | 8695 | 8717 | UUCAGUGUCAGCAGAUACAAUUU | 704 |
| 1689820 | 117614 | 117636 | 8740 | 8762 | UUACUUUGUUACAUACUUAAACU | 705 |
| 1689853 | 117778 | 117800 | 8904 | 8926 | UCACUUUUGCACAACAAGUGACC | 706 |
| 1689862 | 117823 | 117845 | 8949 | 8971 | UUUCUUUGUUUCACAAAAGCAAU | 707 |
| 1689871 | 117868 | 117890 | 8994 | 9016 | UACCAAGAGCGCCUUCCAAUCUA | 708 |
| 1689874 | 117883 | 117905 | 9009 | 9031 | UUGUGGGUUAUUCUUUACCAAGA | 709 |
| 1689889 | 117951 | 117973 | 9077 | 9099 | UGGAGCAACAGUCAGCAUCAUCA | 710 |
| 1689901 | 118011 | 118033 | 9137 | 9159 | UGCUAAGGUUGACAGCAACUUUC | 711 |
| 1689916 | 118096 | 118118 | 9222 | 9244 | UAUCCUAGCCUUCCUCAGCUGAA | 712 |
| 1689925 | 118141 | 118163 | 9267 | 9289 | UGAUUUCUAGCUCGAGCUAGUCU | 713 |
| 1689949 | 118272 | 118294 | 9398 | 9420 | UUUCCACCCUGGUUUGACAGGUA | 714 |
| 1689952 | 118287 | 118309 | 9413 | 9435 | UUGCAUCCCUCUAAAUUUCCACC | 715 |
| 1689955 | 118302 | 118324 | 9428 | 9450 | UGAGACAUACUGUACUUGCAUCC | 716 |
| 1689961 | 118332 | 118354 | 9458 | 9480 | UUCAUAUAAUCAGCAACAGAGAC | 717 |
| 1689964 | 118347 | 118369 | 9473 | 9495 | UACAACAAUUUUAUCUUCAUAUA | 718 |
| 1689973 | 118392 | 118414 | 9518 | 9540 | UGAUUCAAUCACUGUGAAAAUGU | 719 |
| 1689976 | 118407 | 118429 | 9533 | 9555 | UUUUUCUUUUUACAAUGAUUCAA | 720 |
| 1690009 | 118567 | 118589 | 9693 | 9715 | UUUUUGAAGCAGAGAAAACACAU | 721 |
| 1690015 | 118597 | 118619 | 9723 | 9745 | UAAUAGCCUUUAAUACUAAAAUG | 722 |

“Start site” indicates the 5′-most nucleoside to which the antisense RNAi oligonucleotide is complementary in the target nucleic acid sequence. “Stop site” indicates the 3′-most nucleoside to which the antisense RNAi oligonucleotide is complementary in the target nucleic acid sequence. Each antisense RNAi oligonucleoside listed in the Table 8 below is complementary to SEQ ID NO: 1 (ENSEMBL Accession No. ENSG00000165194.15 from version 104: May 2021), to SEQ ID NO: 2 (ENSEMBL Accession No. ENST00000373034.8 from version 104: May 2021), or to both with the exception of a single mismatch at position 1 (from 5′ to 3′) of the antisense RNAi oligonucleotide. “N/A” indicates that the antisense RNAi oligonucleotide has two or more mismatches to that particular target nucleic acid sequence in Table 8 below.

TABLE 8
Design of antisense strand modified oligonucleotides targeted to human PCDH19

| | SEQ ID | SEQ ID | SEQ ID | SEQ ID | | |
|---|---|---|---|---|---|---|
| Antisense | NO: 1 | NO: 1 | NO: 2 | NO: 2 | | |
| Strand | Antisense | Antisense | Antisense | Antisense | | |
| Compound | Strand | Strand | Strand | Strand | | SEQ |
| No. | Start Site | Stop Site | Start Site | Stop Site | Antisense Strand Sequence (5′ to 3′) | ID NO |
| 1688312 | 190 | 211 | 190 | 211 | UUUUCGGAUGAAAUCGCUCAGCC | 723 |
| 1688315 | 205 | 226 | 205 | 226 | UGUAGGAGGUUUAGGAUUUCGGA | 724 |
| 1688318 | 216 | 237 | 216 | 237 | UAUGGGUUUGGGGUAGGAGGUUU | 725 |
| 1688330 | 423 | 444 | 423 | 444 | UCUUGCAGGAGCGUCUUGAUUUC | 726 |
| 1688333 | 430 | 451 | 430 | 451 | UAAGUUUACUUGCAGGAGCGUCU | 727 |
| 1688336 | 445 | 466 | 445 | 466 | UAUUUUGUCGCCGCCGAAGUUUA | 728 |
| 1688342 | 712 | 733 | 712 | 733 | UUUUGUCCAAGAAAAUCAAAGCC | 729 |
| 1688345 | 723 | 744 | 723 | 744 | UGGAGAUGGCAAUUUGUCCAAGA | 730 |
| 1688348 | 738 | 759 | 738 | 759 | UAGUUUGUGGAAACGGGGAGAUG | 731 |
| 1688351 | 748 | 769 | 748 | 769 | UCUCCACGUCAAGUUUGUGGAAA | 732 |
| 1688354 | 770 | 791 | 770 | 791 | UAAGUUAACAACGCAGUCCGACC | 733 |
| 1688360 | 792 | 813 | 792 | 813 | UGGCGUUUUCGUCUAGGAAAUCA | 734 |
| 1688366 | 1259 | 1280 | 1259 | 1280 | UACAGUACUUGAGGCGAAGGUAA | 735 |
| 1688372 | 1370 | 1391 | 1370 | 1391 | UGCUACUCAGUUUUACCCCUUCA | 736 |
| 1688375 | 1733 | 1754 | 1733 | 1754 | UCUUGAGAUUAAUGAGGGCGGCA | 737 |
| 1688378 | 1748 | 1769 | 1748 | 1769 | UCUCUUCUACCGAGUACUUGAGA | 738 |
| 1688384 | 1879 | 1900 | 1879 | 1900 | UUUGAUGUCCACUAGGUGUGGAG | 739 |
| 1688387 | 1888 | 1909 | 1888 | 1909 | UGAGCUGGGAUUGAUGUCCACUA | 740 |
| 1688390 | 1915 | 1936 | 1915 | 1936 | UAUCUUCUGCUUGGUGACCAGCA | 741 |
| 1688393 | 1930 | 1951 | 1930 | 1951 | UAGCAGAUCACGGUCAAUCUUCU | 742 |
| 1688396 | 1932 | 1953 | 1932 | 1953 | UACAGCAGAUCACGGUCAAUCUU | 743 |
| 1688402 | 2002 | 2023 | 2002 | 2023 | UUUUAUCACGCAGAUUUCCAUUG | 744 |
| 1688405 | 2017 | 2038 | 2017 | 2038 | UUCCUUGAUCUCCACCUUUAUCA | 745 |
| 1688408 | 2031 | 2052 | 2031 | 2052 | UCAUUGUCGUUCAGGUCCUUGAU | 746 |
| 1688411 | 2135 | 2156 | 2135 | 2156 | UGCUUCCUGAGUCUGGAUCGUAA | 747 |
| 1688414 | 2137 | 2158 | 2137 | 2158 | UAAGCUUCCUGAGUCUGGAUCGU | 748 |
| 1688420 | 2337 | 2358 | 2337 | 2358 | UCCUUGAUACUAAGGCCAACGGU | 749 |
| 1688426 | 2367 | 2388 | 2367 | 2388 | UACACCGGGUUGUUGUCAUUGGA | 750 |
| 1688429 | 2371 | 2392 | 2371 | 2392 | UCUAAACACCGGGUUGUUGUCAU | 751 |
| 1688453 | 2851 | 2872 | 2851 | 2872 | UUAUUCCUGCAGUCGAAAGGGCA | 752 |
| 1688456 | 2866 | 2887 | 2866 | 2887 | UGUGGAGAAGCUCUCAUAUUCCU | 753 |
| 1688465 | 2931 | 2952 | 2931 | 2952 | UGUGCCUGAAUUGUGAGGUUGUA | 754 |
| 1688474 | 3014 | 3035 | 3014 | 3035 | UAAAGUGCGGGUGGUUGUCAUUU | 755 |
| 1688477 | 3015 | 3036 | 3015 | 3036 | UAAAAGUGCGGGUGGUUGUCAUU | 756 |
| 1688486 | 3055 | 3076 | 3055 | 3076 | UGUGUUGUUCUCCUGCACAAUGA | 757 |
| 1688489 | 3171 | 3192 | 3171 | 3192 | UCAUAGGUGAAGACAGGCAUGUC | 758 |
| 1688516 | 3423 | 3444 | 3423 | 3444 | UCAACAGUCACCAGGUAGCCUAU | 759 |
| 1688525 | 3455 | 3476 | 3455 | 3476 | UAUUUUCGCCCUCAUCGUAGUCU | 760 |
| 1688534 | 3525 | 3546 | 3525 | 3546 | UCUUCGCCAUUGACCUGGUCUAU | 761 |
| 1688543 | 3638 | 3659 | 3638 | 3659 | UGUAGAUUAGGACGAGAGCAGAG | 762 |
| 1688546 | 3652 | 3673 | 3652 | 3673 | UAGAGCAGGGGACAAGUAGAUUA | 763 |
| 1688552 | 3696 | 3717 | 3696 | 3717 | UAAAUCAAGGACAAGUUCACAGA | 764 |
| 1688555 | 3711 | 3732 | 3711 | 3732 | UCCAGGGCAAUAAUGAAAAUCAA | 765 |
| 1688558 | 3718 | 3739 | 3718 | 3739 | UAUGGAGCCCAGGGCAAUAAUGA | 766 |
| 1688564 | 3752 | 3773 | 3752 | 3773 | UCACGAAGAUCAUAGUUACAAAG | 767 |
| 1688567 | 3766 | 3787 | 3766 | 3787 | UUUGCACUUGAUUGCCACGAAGA | 768 |
| 1688570 | 3781 | 3802 | 3781 | 3802 | UUCUUUGUUGUCUCGCUUGCACU | 769 |
| 1688573 | 3796 | 3817 | 3796 | 3817 | UUUGUAGGUCCGGAUCUCUUUGU | 770 |
| 1688579 | 6612 | 6633 | 3826 | 3847 | UAGACAAGUGAUGGUUAAACAAU | 771 |
| 1688585 | 6642 | 6663 | 3856 | 3877 | UCUGUUUUGUCCUUUUAUAAAAC | 772 |
| 1688588 | 6657 | 6678 | 3871 | 3892 | UCAAUGCAGACACUUGCUGUUUU | 773 |
| 1688591 | 6670 | 6691 | 3884 | 3905 | UAGAAACCGAGAUGCAAUGCAGA | 774 |
| 1688600 | 6710 | 6731 | 3924 | 3945 | UCUUUCUCCUCUGUCUUUUUGUC | 775 |
| 1688609 | 7425 | 7446 | 3967 | 3988 | UUGCCCAUAGGAGUACUCAGCAA | 776 |
| 1688612 | 7440 | 7461 | 3982 | 4003 | UCUUGAUUUCUUUUGAUGCCCAU | 777 |
| 1688615 | 7455 | 7476 | 3997 | 4018 | UAUUUUUUUCUUCUUGCUUGAUU | 778 |
| 1688618 | 7470 | 7491 | 4012 | 4033 | UAUGUCAUUCUUACUGAUUUUUU | 779 |
| 1688621 | 7480 | 7501 | 4022 | 4043 | UUACCAGGCGGAUGUCAUUCUUA | 780 |
| 1688624 | 7508 | 7529 | 4050 | 4071 | UUCUUGUCUGUCUCCUCCACAUC | 781 |
| 1688944 | 7523 | 7544 | 4065 | 4086 | UAACUGACAACGUUCAUCUUGUC | 782 |
| 1688947 | 7532 | 7553 | 4074 | 4095 | UGGGAAGAGCAACUGACAACGUU | 783 |
| 1688950 | 7553 | 7574 | 4095 | 4116 | UAAUAGUUGAGGGAGGAGGUCAG | 784 |
| 1688956 | 7572 | 7593 | 4114 | 4135 | UGUCUGCUGGUGGUAGUCAAAAU | 785 |
| 1688959 | 7614 | 7635 | 4156 | 4177 | UUUCAGGAAAGUGCUCUCAGAGC | 786 |
| 1688962 | 7629 | 7650 | 4171 | 4192 | UUUCUGGUUCUCCACAUUCAGGA | 787 |
| 1688965 | 7634 | 7655 | 4176 | 4197 | UGGGUAUUCUGGUUCUCCACAUU | 788 |
| 1688977 | 7680 | 7701 | 4222 | 4243 | UUGGCUGUUGAAAGAGUGAUGGU | 789 |
| 1688983 | 7722 | 7743 | 4264 | 4285 | UAGAGGCACACCGUUGAUAAUCA | 790 |
| 1688992 | 59587 | 59608 | 4310 | 4331 | UGCUAUUCACGUAGUUGGAGUCA | 791 |
| 1688998 | N/A | N/A | 4340 | 4361 | UGGUGGAGCUGCUCUUGAUUAAA | 792 |
| 1689004 | 68209 | 68230 | 4362 | 4383 | UUGUUGCCCUCUAAGUCCUUGAA | 793 |
| 1689010 | 68240 | 68261 | 4393 | 4414 | UUCACUCUCCUCAUGUCCACUAU | 794 |
| 1689013 | 68255 | 68276 | 4408 | 4429 | UUCACUGUCAGUUUGGUCACUCU | 795 |
| 1689019 | 68288 | 68309 | 4441 | 4462 | UGUAUCACAAUACAGGCUCCGCU | 796 |
| 1689022 | 68303 | 68324 | 4456 | 4477 | UACAUCGUUGACAGCAGUAUCAC | 797 |
| 1689025 | 68317 | 68338 | 4470 | 4491 | UCACUGGUGUUCAGCACAUCGUU | 798 |
| 1689031 | N/A | N/A | 4515 | 4536 | UCUUCAUUCUGAUCAUGAUCAGG | 799 |
| 1689037 | 113412 | 113433 | 4538 | 4559 | UGCAUUCUUCCCGGCAAUGAAAU | 800 |
| 1689040 | 113500 | 113521 | 4626 | 4647 | UUGAUGUUCCUCACAUGCUCAGG | 801 |
| 1689049 | 113534 | 113555 | 4660 | 4681 | UACAUCAGCAGCAGUAGCUUCAA | 802 |
| 1689052 | 113550 | 113571 | 4676 | 4697 | UGUCGUCAUAAGCCUCGACAUCA | 803 |
| 1689055 | 113581 | 113602 | 4707 | 4728 | UAGGUUGCGAAAGUCCGUUUGGU | 804 |
| 1689058 | 113596 | 113617 | 4722 | 4743 | UUGACAUCUUUCCCAAAGGUUGC | 805 |
| 1689064 | 113648 | 113669 | 4774 | 4795 | UUCGACAGUCCUCUUGCCUUUCA | 806 |
| 1689067 | 113656 | 113677 | 4782 | 4803 | UUGGUCACAUCGACAGUCCUCUU | 807 |
| 1689070 | 113782 | 113803 | 4908 | 4929 | UUGGUGUAAGACACGGAAGGCUU | 808 |
| 1689073 | 113783 | 113804 | 4909 | 4930 | UAUGGUGUAAGACACGGAAGGCU | 809 |
| 1689079 | 113839 | 113860 | 4965 | 4986 | UCAUUGUUGACAUUGUUGACAUA | 810 |
| 1689082 | 113849 | 113870 | 4975 | 4996 | UCGAGUAGGGCCAUUGUUGACAU | 811 |
| 1689088 | 113906 | 113927 | 5032 | 5053 | UCUGACCUCAUGCAUGACUUUCU | 812 |
| 1689094 | 113938 | 113959 | 5064 | 5085 | UACUCUUUGUUGCGACCUUCCUU | 813 |
| 1689100 | 113965 | 113986 | 5091 | 5112 | UUAUCCUUCAGACGCUUCACACC | 814 |
| 1689103 | 113980 | 114001 | 5106 | 5127 | UUGGUUUAGAGAACGAUAUCCUU | 815 |
| 1689109 | 114010 | 114031 | 5136 | 5157 | UGUGUGGUUUCUUUCUCUUCUUC | 816 |
| 1689112 | 114024 | 114045 | 5150 | 5171 | UUUCUUCACUAGCCAGUGUGGUU | 817 |
| 1689115 | 114039 | 114060 | 5165 | 5186 | UACAAGAAGCUCCUGCUUCUUCA | 818 |
| 1689121 | 114069 | 114090 | 5195 | 5216 | UUCAAGAACCAACCAUUGGUGAG | 819 |
| 1689127 | 114099 | 114120 | 5225 | 5246 | UCAUUUAGGAAAAGCUCUGAAAU | 820 |
| 1689133 | 114129 | 114150 | 5255 | 5276 | UUCACAGAAUGAUAAUCACCUAU | 821 |
| 1689142 | 114175 | 114196 | 5301 | 5322 | UUUUGCUCCAACUGAACACCCCU | 822 |
| 1689145 | 114190 | 114211 | 5316 | 5337 | UUCAAGCCAAAGCUAAUUUGCUC | 823 |
| 1689148 | 114202 | 114223 | 5328 | 5349 | UCCCAUGAACAACUCAAGCCAAA | 824 |
| 1689151 | 114213 | 114234 | 5339 | 5360 | UAACAUCAAGGCCCCAUGAACAA | 825 |
| 1689160 | 114258 | 114279 | 5384 | 5405 | UUUAAUACAUAAUACUUUUCACA | 826 |
| 1689163 | 114273 | 114294 | 5399 | 5420 | UUAUAAAUUCAAACACUUAAUAC | 827 |
| 1689172 | 114317 | 114338 | 5443 | 5464 | UACAAACAAUGGUAAUUUAUAUU | 828 |
| 1689181 | 114362 | 114383 | 5488 | 5509 | UAGAGCUUUUUUUUUUCACUUUU | 829 |
| 1689184 | 114377 | 114398 | 5503 | 5524 | UUUAUUAAAGGUGUCCAGAGCUU | 830 |
| 1689187 | 114392 | 114413 | 5518 | 5539 | UAAAACAGUGGCGAGCUUAUUAA | 831 |
| 1689190 | 114407 | 114428 | 5533 | 5554 | UUUGUCUACACUACAAAAAACAG | 832 |
| 1689193 | 114422 | 114443 | 5548 | 5569 | UUAAAUGACCAUUAACUUGUCUA | 833 |
| 1689196 | 114437 | 114458 | 5563 | 5584 | UAUGAAUAGUACACAAUAAAUGA | 834 |
| 1689205 | 114482 | 114503 | 5608 | 5629 | UUUCAGAAACAACCUUUUGGGGG | 835 |
| 1689211 | 114511 | 114532 | 5637 | 5658 | UAAAGUAGCGGCAUUGGGAAGUA | 836 |
| 1689217 | 114549 | 114570 | 5675 | 5696 | UGCUAGCAAAAGGGUUUUUAAAG | 837 |
| 1689220 | 114564 | 114585 | 5690 | 5711 | UUACAACAAUAGCACAGCUAGCA | 838 |
| 1689223 | 114579 | 114600 | 5705 | 5726 | UAUUUGCAAUAUCAAAUACAACA | 839 |
| 1689226 | 114594 | 114615 | 5720 | 5741 | UACCACACACAUAGCAAUUUGCA | 840 |
| 1689229 | 114609 | 114630 | 5735 | 5756 | UAAGCCUUUUGCCAUCACCACAC | 841 |
| 1689232 | 114624 | 114645 | 5750 | 5771 | UAACACCAACUUUUAAAAGCCUU | 842 |
| 1689241 | 114669 | 114690 | 5795 | 5816 | UACUUUGUUUUUGUCUGUAGUUU | 843 |
| 1689250 | 114714 | 114735 | 5840 | 5861 | UUAUAUGCAUGGUGUUGCUCAUU | 844 |
| 1689253 | 114729 | 114750 | 5855 | 5876 | UUCCAAGUACUCAAUCUAUAUGC | 845 |
| 1689256 | 114744 | 114765 | 5870 | 5891 | UAAAACGUGAGUAUUCUCCAAGU | 846 |
| 1689262 | 114774 | 114795 | 5900 | 5921 | UAAAGUGGUGAGCAAUCACUACA | 847 |
| 1689265 | 114789 | 114810 | 5915 | 5936 | UGGAAAAAUACUUUGCAAAGUGG | 848 |
| 1689268 | 114804 | 114825 | 5930 | 5951 | UGACCACUGAAAGGCAGGAAAAA | 849 |
| 1689271 | 114805 | 114826 | 5931 | 5952 | UAGACCACUGAAAGGCAGGAAAA | 850 |
| 1689274 | 114826 | 114847 | 5952 | 5973 | UUGAUCUAUCAUGGACCUGGGCA | 851 |
| 1689277 | 114841 | 114862 | 5967 | 5988 | UAAUGACUUUCUGCAAUGAUCUA | 852 |
| 1689283 | 114871 | 114892 | 5997 | 6018 | UAACUGCAAAUUAGAUAGCAGCC | 853 |
| 1689286 | 114886 | 114907 | 6012 | 6033 | UCACAGUAUUUCUGACAACUGCA | 854 |
| 1689289 | 114901 | 114922 | 6027 | 6048 | UUCAUAAAACUUUCAGCACAGUA | 855 |
| 1689292 | 114916 | 114937 | 6042 | 6063 | UUUUAAAAGAUGGAUGUCAUAAA | 856 |
| 1689295 | 114931 | 114952 | 6057 | 6078 | UUUCAGGGCCUGAAAAUUUAAAA | 857 |
| 1689298 | 114940 | 114961 | 6066 | 6087 | UACUUUCCUGUUCAGGGCCUGAA | 858 |
| 1689301 | 114955 | 114976 | 6081 | 6102 | UAUAACUUCAGGAGCAACUUUCC | 859 |
| 1689313 | 115015 | 115036 | 6141 | 6162 | UUAGAUUCAGUCAUCUUCAAUUU | 860 |
| 1689316 | 115030 | 115051 | 6156 | 6177 | UAUUUAGAUCUUAAAAUAGAUUC | 861 |
| 1689319 | 115045 | 115066 | 6171 | 6192 | UUCUGAAGAGAUGCUAAUUUAGA | 862 |
| 1689322 | 115060 | 115081 | 6186 | 6207 | UAUCAGGAAGUGUGUGUCUGAAG | 863 |
| 1689325 | 115075 | 115096 | 6201 | 6222 | UAUUGGGAAACACUGAAUCAGGA | 864 |
| 1689334 | 115108 | 115129 | 6234 | 6255 | UCAUGACGUGCUUUGCAGUCUAA | 865 |
| 1689337 | 115131 | 115152 | 6257 | 6278 | UACAUGGAGUUCUCAAUCCCAGC | 866 |
| 1689340 | 115146 | 115167 | 6272 | 6293 | UCUAAGAUACAGAGCAACAUGGA | 867 |
| 1689343 | 115161 | 115182 | 6287 | 6308 | UCCUACGUAUAUAAGCCUAAGAU | 868 |
| 1689349 | 115191 | 115212 | 6317 | 6338 | UAAAUAUACACUUUGUACUGCUU | 869 |
| 1689355 | 115221 | 115242 | 6347 | 6368 | UGAUGCUGUUACCUGUUACCUGU | 870 |
| 1689358 | 115236 | 115257 | 6362 | 6383 | UAGUAAUUGGAUUAGCGAUGCUG | 871 |
| 1689364 | 115266 | 115287 | 6392 | 6413 | UAGUGUCUGAGUGUCUCCGUUUA | 872 |
| 1689370 | 115296 | 115317 | 6422 | 6443 | UAAAACUAUUAGUCAUAAAAAGA | 873 |
| 1689379 | 115341 | 115362 | 6467 | 6488 | UAAAGGAUUUUUAUUUUAGUAUU | 874 |
| 1689382 | 115356 | 115377 | 6482 | 6503 | UGAGUUGGCUAAAUCGAAAGGAU | 875 |
| 1689388 | 115386 | 115407 | 6512 | 6533 | UAAAGGGUGUAUUCCUAGCCUAC | 876 |
| 1689391 | 115401 | 115422 | 6527 | 6548 | UAGUGUGUUAAUUUAAAAAGGGU | 877 |
| 1689394 | 115416 | 115437 | 6542 | 6563 | UAAAAAAAGGAUCUACAGUGUGU | 878 |
| 1689397 | 115431 | 115452 | 6557 | 6578 | UGGAAUGUUAAAACAGAAAAAAA | 879 |
| 1689400 | 115446 | 115467 | 6572 | 6593 | UAAUGGGGGAGUUAGAGGAAUGU | 880 |
| 1689412 | 115506 | 115527 | 6632 | 6653 | UAUGAACUCUUAGGCUGCAAAAA | 881 |
| 1689415 | 115521 | 115542 | 6647 | 6668 | UGUUUAUUUUUAAAUAAUGAACU | 882 |
| 1689418 | 115536 | 115557 | 6662 | 6683 | UUGACAUAUUUCUUUAGUUUAUU | 883 |
| 1689421 | 115551 | 115572 | 6677 | 6698 | UAAGGCAGAACAAAGGUGACAUA | 884 |
| 1689424 | 115566 | 115587 | 6692 | 6713 | UAACUCUCUUAAGACCAAGGCAG | 885 |
| 1689430 | 115596 | 115617 | 6722 | 6743 | UCAGAACCAUAAAACUUGUUUCU | 886 |
| 1689433 | 115611 | 115632 | 6737 | 6758 | UUGAAACAAAUGAAAACAGAACC | 887 |
| 1689436 | 115626 | 115647 | 6752 | 6773 | UCUGAAUUUCAAAAAAUGAAACA | 888 |
| 1689448 | 115686 | 115707 | 6812 | 6833 | UAAAAUAAAUCCUCUUUUGCAUU | 889 |
| 1689454 | 115716 | 115737 | 6842 | 6863 | UAAAGAGCUGACUUAUCUACCUA | 890 |
| 1689457 | 115731 | 115752 | 6857 | 6878 | UUAAGUAAAACAUUCAAAAGAGC | 891 |
| 1689460 | 115746 | 115767 | 6872 | 6893 | UGAACUCAGGCAAAAAUAAGUAA | 892 |
| 1689466 | 115776 | 115797 | 6902 | 6923 | UGAAAAUAUUCCUAGUAAUGCGU | 893 |
| 1689475 | 115853 | 115874 | 6979 | 7000 | UAAUGUUCUCCCCUACCUAACCC | 894 |
| 1689484 | 115898 | 115919 | 7024 | 7045 | UGAAAUAAGGAUGGAUUUGUAGC | 895 |
| 1689487 | 115913 | 115934 | 7039 | 7060 | UUUCAGCACUGAAUAAGAAAUAA | 896 |
| 1689493 | 115943 | 115964 | 7069 | 7090 | UAGCCACACCAAUAUUUUGCAUG | 897 |
| 1689505 | 116003 | 116024 | 7129 | 7150 | UAAAAAUGAAGAUGUUAUGGUAA | 898 |
| 1689508 | 116018 | 116039 | 7144 | 7165 | UGGCAAUGAAAUGAGGAAAAAUG | 899 |
| 1689511 | 116033 | 116054 | 7159 | 7180 | UGAAAACUGUAUGAGAGGCAAUG | 900 |
| 1689514 | 116048 | 116069 | 7174 | 7195 | UAAUAAAUCAGAGGGAGAAAACU | 901 |
| 1689520 | 116078 | 116099 | 7204 | 7225 | UUACUUAAUUCAAAAUCCACUAC | 902 |
| 1689526 | 116108 | 116129 | 7234 | 7255 | UAUCAAAUAGUCACUUGCAAAGA | 903 |
| 1689529 | 116123 | 116144 | 7249 | 7270 | UAUUUUUAAUGUGUUAAUCAAAU | 904 |
| 1689532 | 116138 | 116159 | 7264 | 7285 | UGGAAAUUUUUAAAAAAUUUUUA | 905 |
| 1689535 | 116153 | 116174 | 7279 | 7300 | UAUCAUAACUUAAAGGGGAAAUU | 906 |
| 1689538 | 116168 | 116189 | 7294 | 7315 | UAUUCUAUAGGCAGCCAUCAUAA | 907 |
| 1689541 | 116183 | 116204 | 7309 | 7330 | UUGAGAAACAGUCUAAAUUCUAU | 908 |
| 1689547 | 116213 | 116234 | 7339 | 7360 | UCUAAUAACAUAAUAUCAGGAUG | 909 |
| 1689550 | 116228 | 116249 | 7354 | 7375 | UACCUUGAAAUCGUAGCUAAUAA | 910 |
| 1689553 | 116243 | 116264 | 7369 | 7390 | UUCUGACUUCAAAGUGACCUUGA | 911 |
| 1689556 | 116258 | 116279 | 7384 | 7405 | UUAGAAAACUGUGAAGUCUGACU | 912 |
| 1689559 | 116273 | 116294 | 7399 | 7420 | UCAAGAAAUACACCUGUAGAAAA | 913 |
| 1689565 | 116289 | 116310 | 7415 | 7436 | UUUCCCACUGAGCAUAGCAAGAA | 914 |
| 1689568 | 116307 | 116328 | 7433 | 7454 | UAUCUUUUCUGCUCCCUGGUUCC | 915 |
| 1689571 | 116322 | 116343 | 7448 | 7469 | UUUCCCACUUUCCACAAUCUUUU | 916 |
| 1689574 | 116337 | 116358 | 7463 | 7484 | UACGUGACAAUUAAUGUUCCCAC | 917 |
| 1689577 | 116347 | 116368 | 7473 | 7494 | UCUCAACAGCCACGUGACAAUUA | 918 |
| 1689580 | 116389 | 116410 | 7515 | 7536 | UAACUUGGCUGGUAAGAGGCAUG | 919 |
| 1689583 | 116404 | 116425 | 7530 | 7551 | UACUCUAACCAUUUAAAACUUGG | 920 |
| 1689586 | 116419 | 116440 | 7545 | 7566 | UAUCAAUGCCAAUGCCACUCUAA | 921 |
| 1689589 | 116434 | 116455 | 7560 | 7581 | UCAAGAGAAAAGCAAAAUCAAUG | 922 |
| 1689592 | 116449 | 116470 | 7575 | 7596 | UGAAAUGAAGAAAACCCAAGAGA | 923 |
| 1689595 | 116464 | 116485 | 7590 | 7611 | UGAUGAAUUAUGAAAAGAAAUGA | 924 |
| 1689598 | 116479 | 116500 | 7605 | 7626 | UGAUCUAUAGCUUGAAGAUGAAU | 925 |
| 1689601 | 116494 | 116515 | 7620 | 7641 | UUAGGGAAGAAGGAUGGAUCUAU | 926 |
| 1689604 | 116509 | 116530 | 7635 | 7656 | UAACAAAGUAUUUGGAUAGGGAA | 927 |
| 1689610 | 116538 | 116559 | 7664 | 7685 | UAAGCCAAAAAAAUCUGACUGCC | 928 |
| 1689613 | 116552 | 116573 | 7678 | 7699 | UUCAGUGAGGUUUGGAAGCCAAA | 929 |
| 1689616 | 116567 | 116588 | 7693 | 7714 | UAUUCUCUGAACAUGCUCAGUGA | 930 |
| 1689625 | 116612 | 116633 | 7738 | 7759 | UAUACAUCAGACAAUUGUGGGGA | 931 |
| 1689628 | 116627 | 116648 | 7753 | 7774 | UCUUCUGAGAGGACUCAUACAUC | 932 |
| 1689631 | 116642 | 116663 | 7768 | 7789 | UUAGUAUGCACUUGUGCUUCUGA | 933 |
| 1689634 | 116657 | 116678 | 7783 | 7804 | UUUUACAAAGCCUUCCUAGUAUG | 934 |
| 1689637 | 116672 | 116693 | 7798 | 7819 | UUGCCUACAAAACCAGUUUACAA | 935 |
| 1689643 | 116693 | 116714 | 7819 | 7840 | UUGCCUAUUUGCAGGUACAGCCU | 936 |
| 1689646 | 116708 | 116729 | 7834 | 7855 | UGAUCCUUGCUCAGAAUGCCUAU | 937 |
| 1689649 | 116723 | 116744 | 7849 | 7870 | UCAAAGGGAAAUUUAGGAUCCUU | 938 |
| 1689652 | 116733 | 116754 | 7859 | 7880 | UGGACAUGAGCCAAAGGGAAAUU | 939 |
| 1689655 | 116768 | 116789 | 7894 | 7915 | UCAUGCCAAAAAGGAGAAGGUGG | 940 |
| 1689658 | 116783 | 116804 | 7909 | 7930 | UUAGCUUUUCACCAGACAUGCCA | 941 |
| 1689661 | 116797 | 116818 | 7923 | 7944 | UCCCUGUGAACCUUGUAGCUUUU | 942 |
| 1689664 | 116812 | 116833 | 7938 | 7959 | UAGGGAGAAUAUUUGGCCCUGUG | 943 |
| 1689667 | 116827 | 116848 | 7953 | 7974 | UUGUUUAGAGAACAAAAGGGAGA | 944 |
| 1689670 | 116842 | 116863 | 7968 | 7989 | UUUUAUGUGUGCAACAUGUUUAG | 945 |
| 1689673 | 116857 | 116878 | 7983 | 8004 | UAAUCACUCCCUUGAGUUUAUGU | 946 |
| 1689676 | 116872 | 116893 | 7998 | 8019 | UGGACUUAGGAACCGAAAUCACU | 947 |
| 1689679 | 116887 | 116908 | 8013 | 8034 | UGAAUAGAACUCCCUAGGACUUA | 948 |
| 1689682 | 116902 | 116923 | 8028 | 8049 | UACAUCAGAAUAAAUGGAAUAGA | 949 |
| 1689685 | 116917 | 116938 | 8043 | 8064 | UUCCUCCUUAAAAGGGACAUCAG | 950 |
| 1689688 | 116932 | 116953 | 8058 | 8079 | UCACUUCAACACCAACUCCUCCU | 951 |
| 1689703 | 117007 | 117028 | 8133 | 8154 | UCCUUAGGUGCAAUUUUGGAUGA | 952 |
| 1689709 | 117037 | 117058 | 8163 | 8184 | UACUAAGCGAACAAUUUUGUCUA | 953 |
| 1689715 | 117067 | 117088 | 8193 | 8214 | UAAAAUAUUUCUGAUUAGAGAUC | 954 |
| 1689730 | 117137 | 117158 | 8263 | 8284 | UCUGAAGUAACACUAUCAAGAUG | 955 |
| 1689733 | 117152 | 117173 | 8278 | 8299 | UUCAGUCAGUCAUAUGCUGAAGU | 956 |
| 1689736 | 117167 | 117188 | 8293 | 8314 | UACUCCCUUCAUAAAAUCAGUCA | 957 |
| 1689739 | 117182 | 117203 | 8308 | 8329 | UGGAAAAGUCAGCUAAACUCCCU | 958 |
| 1689745 | 117212 | 117233 | 8338 | 8359 | UCUUUUUGUUUUUAAUAUGAUAG | 959 |
| 1689751 | 117240 | 117261 | 8366 | 8387 | UCUUUGUCCUUGCUGCUUCAAGU | 960 |
| 1689760 | 117314 | 117335 | 8440 | 8461 | UUACAAUAAUAAUCCUUUUCCAC | 961 |
| 1689763 | 117329 | 117350 | 8455 | 8476 | UAGAACUGCUAUUAUGUACAAUA | 962 |
| 1689766 | 117344 | 117365 | 8470 | 8491 | UAUUGGAAUUCCAGCCAGAACUG | 963 |
| 1689769 | 117359 | 117380 | 8485 | 8506 | UAUCCCCAGAAAGUGGAUUGGAA | 964 |
| 1689772 | 117374 | 117395 | 8500 | 8521 | UUUCAACAUCAAAAUGAUCCCCA | 965 |
| 1689775 | 117389 | 117410 | 8515 | 8536 | UUGCUUUGGGGGAACAUUCAACA | 966 |
| 1689778 | 117404 | 117425 | 8530 | 8551 | UCCAAACUUGUCAGAAUGCUUUG | 967 |
| 1689787 | 117449 | 117470 | 8575 | 8596 | UAAAGUCAAUAAGGGUCUUAUUA | 968 |
| 1689793 | 117479 | 117500 | 8605 | 8626 | UAUUUCUUAAGCUCAUUUAUGCC | 969 |
| 1689796 | 117494 | 117515 | 8620 | 8641 | UGACUGUAUACAAUGGAUUUCUU | 970 |
| 1689799 | 117509 | 117530 | 8635 | 8656 | UGAAGGCAAACACUAGGACUGUA | 971 |
| 1689802 | 117524 | 117545 | 8650 | 8671 | UAUGUAAUUUUACCAAGAAGGCA | 972 |
| 1689805 | 117539 | 117560 | 8665 | 8686 | UAAAUGACAUCCUUAAAUGUAAU | 973 |
| 1689814 | 117584 | 117605 | 8710 | 8731 | UACAUCUCAAUACACUUCAGUGU | 974 |
| 1689817 | 117599 | 117620 | 8725 | 8746 | UUUAAACUAAAGAACAACAUCUC | 975 |
| 1689823 | 117629 | 117650 | 8755 | 8776 | UAUCCCCUUUGAAUAUUACUUUG | 976 |
| 1689826 | 117638 | 117659 | 8764 | 8785 | UCCCCCAAACAUCCCCUUUGAAU | 977 |
| 1689829 | 117668 | 117689 | 8794 | 8815 | UUCAAUAUAAAGCCACCCGGUCC | 978 |
| 1689832 | 117683 | 117704 | 8809 | 8830 | UUGGAAUAUUUCAUUAUCAAUAU | 979 |
| 1689835 | 117698 | 117719 | 8824 | 8845 | UCAAUGUGUUUUGCUCUGGAAUA | 980 |
| 1689838 | 117713 | 117734 | 8839 | 8860 | UACAACUUCAUUAGGACAAUGUG | 981 |
| 1689841 | 117728 | 117749 | 8854 | 8875 | UACGAAGAGAGCUGCAACAACUU | 982 |
| 1689844 | 117741 | 117762 | 8867 | 8888 | UGCGGCACAAGAAAACGAAGAGA | 983 |
| 1689847 | 117749 | 117770 | 8875 | 8896 | UCAAUUCCAGCGGCACAAGAAAA | 984 |
| 1689850 | 117763 | 117784 | 8889 | 8910 | UAGUGACCCACAAGCCAAUUCCA | 985 |
| 1689856 | 117793 | 117814 | 8919 | 8940 | UUAUUUGAAAUUAAUUCACUUUU | 986 |
| 1689859 | 117808 | 117829 | 8934 | 8955 | UAAGCAAUAUGAUUCCUAUUUGA | 987 |
| 1689865 | 117838 | 117859 | 8964 | 8985 | UACAAGGUGGUAAAUUUUCUUUG | 988 |
| 1689868 | 117853 | 117874 | 8979 | 9000 | UCAAUCUAUGUAUCAGACAAGGU | 989 |
| 1689877 | 117898 | 117919 | 9024 | 9045 | UUCAAGAUCAGGUGUUUGUGGGU | 990 |
| 1689880 | 117913 | 117934 | 9039 | 9060 | UUACAAUGCUUUCCAAUCAAGAU | 991 |
| 1689883 | 117928 | 117949 | 9054 | 9075 | UUUUUCCCUAAUGACCUACAAUG | 992 |
| 1689886 | 117943 | 117964 | 9069 | 9090 | UAGUCAGCAUCAUCAGUUUUCCC | 993 |
| 1689892 | 117966 | 117987 | 9092 | 9113 | UAUUGCCAACACAGCUGGAGCAA | 994 |
| 1689895 | 117981 | 118002 | 9107 | 9128 | UACAAGGUCAUCUUGAAUUGCCA | 995 |
| 1689898 | 117996 | 118017 | 9122 | 9143 | UAACUUUCAGUUAGUAACAAGGU | 996 |
| 1689904 | 118026 | 118047 | 9152 | 9173 | UUUAGCUGGUUUGUUUGCUAAGG | 997 |
| 1689907 | 118041 | 118062 | 9167 | 9188 | UAGUGCUGUAAUGGUGUUAGCUG | 998 |
| 1689910 | 118056 | 118077 | 9182 | 9203 | UAAAAUCUACUUAACCAGUGCUG | 999 |
| 1689913 | 118071 | 118092 | 9197 | 9218 | UCAGCCAGUGCCAGUAAAAAUCU | 1000 |
| 1689919 | 118111 | 118132 | 9237 | 9258 | UGUCAUUAAAGUCUGUAUCCUAG | 1001 |
| 1689922 | 118126 | 118147 | 9252 | 9273 | UCUAGUCUGGCUCUUGGUCAUUA | 1002 |
| 1689928 | 118156 | 118177 | 9282 | 9303 | UGAGGUAUUUGUUGCUGAUUUCU | 1003 |
| 1689931 | 118171 | 118192 | 9297 | 9318 | UUUCAAAUAGCACAAGGAGGUAU | 1004 |
| 1689934 | 118186 | 118207 | 9312 | 9333 | UACUAAUACCUUAGCAUUCAAAU | 1005 |
| 1689937 | 118201 | 118222 | 9327 | 9348 | UCGUAAGCCUAUUGCAACUAAUA | 1006 |
| 1689940 | 118205 | 118226 | 9331 | 9352 | UAAGGCGUAAGCCUAUUGCAACU | 1007 |
| 1689943 | 118242 | 118263 | 9368 | 9389 | UUAGUGUCAUCAGGCUCAUUCCC | 1008 |
| 1689946 | 118257 | 118278 | 9383 | 9404 | UACAGGUAAACAGUAAUAGUGUC | 1009 |
| 1689958 | 118317 | 118338 | 9443 | 9464 | UCAGAGACUGCUUAAUGAGACAU | 1010 |
| 1689967 | 118362 | 118383 | 9488 | 9509 | UUACAGAUCAGCAUAUACAACAA | 1011 |
| 1689970 | 118377 | 118398 | 9503 | 9524 | UAAAAUGUACAUAACAUACAGAU | 1012 |
| 1689979 | 118422 | 118443 | 9548 | 9569 | UAUUCUUUUUUGUCUUUUUUCUU | 1013 |
| 1689982 | 118437 | 118458 | 9563 | 9584 | UAAAUAGACAGUGGUGAUUCUUU | 1014 |
| 1689985 | 118452 | 118473 | 9578 | 9599 | UUUUAUCAUCUUCAUAAAAUAGA | 1015 |
| 1689988 | 118467 | 118488 | 9593 | 9614 | UAUUUAAUAAAGCUGCUUUAUCA | 1016 |
| 1689991 | 118482 | 118503 | 9608 | 9629 | UAACAUAACUGUAAUAAUUUAAU | 1017 |
| 1689994 | 118497 | 118518 | 9623 | 9644 | UGAUAACACCAUUUGAAACAUAA | 1018 |
| 1689997 | 118512 | 118533 | 9638 | 9659 | UGGAAACAAUGUGGCAGAUAACA | 1019 |
| 1690000 | 118527 | 118548 | 9653 | 9674 | UAACCACCAGAUAAAAGGAAACA | 1020 |
| 1690003 | 118537 | 118558 | 9663 | 9684 | UGGAAACAGAGAACCACCAGAUA | 1021 |
| 1690006 | 118552 | 118573 | 9678 | 9699 | UAACACAUUUAAAGGGGGAAACA | 1022 |
| 1690012 | 118582 | 118603 | 9708 | 9729 | UUAAAAUGCAUUAAAUUUUUGAA | 1023 |
| 1690018 | 114806 | 114827 | 5932 | 5953 | UCAGACCACUGAAAGGCAGGAAA | 1024 |
| 1690021 | 115835 | 115856 | 6961 | 6982 | UACCCCAUCCCUACUGCCUUACC | 1025 |
| 1690024 | 116763 | 116784 | 7889 | 7910 | UCAAAAAGGAGAAGGUGGGGUGG | 1026 |
| 1690027 | 117647 | 117668 | 8773 | 8794 | UCCAGCCCCACCCCCAAACAUCC | 1027 |
| 1690030 | 118230 | 118251 | 9356 | 9377 | UGCUCAUUCCCCUCACUAGAGGA | 1028 |

The sense RNAi oligonucleotide in each case is 21 nucleosides in length; has a sugar motif (from 5′ to 3′) of: yyyyyyfyfffyyyyyyyyyy; wherein “y” represents a 2′-O-methylribosyl sugar and the “f” represents a 2′-fluororibosyl sugar; and an internucleoside linkage motif (from 5′ to 3′) of: ssooooooooooooooooss; wherein ‘o’ represents a phosphodiester internucleoside linkage and ‘s’ represents a phosphorothioate internucleoside linkage. The sense RNAi oligonucleotides are listed below in Table 9.

Each antisense RNAi oligonucleotide is complementary to the target nucleic acid (PCDH19), and each sense RNAi oligonucleotides is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligonucleotides and siRNAs are listed in Table 9 below.

TABLE 9
Design of sense strand modified oligonucleotides of siRNAs targeted to human PCDH19

| | Antisense | Sense | | |
|---|---|---|---|---|
| Duplex | Strand | Strand | | SEQ |
| Compound | Compound | Compound | | ID |
| Number | No. | No. | Sense Strand Sequence (5′ to 3′) | NO |
| 1688314 | 1688312 | 1688313 | CUGAGCGAUUUCAUCCGAAAA | 1029 |
| 1688317 | 1688315 | 1688316 | CGAAAUCCUAAACCUCCUACA | 1030 |
| 1688320 | 1688318 | 1688319 | ACCUCCUACCCCAAACCCAUA | 1031 |
| 1688323 | 1688321 | 1688322 | ACAGACAGAGAAAGCGAGACA | 1032 |
| 1688326 | 1688324 | 1688325 | AGACAGAGAAAGCGAGACAGA | 1033 |
| 1688329 | 1688327 | 1688328 | UCUCCGGGAAGAUGAAAUCAA | 1034 |
| 1688332 | 1688330 | 1688331 | AAUCAAGACGCUCCUGCAAGA | 1035 |
| 1688335 | 1688333 | 1688334 | ACGCUCCUGCAAGUAAACUUA | 1036 |
| 1688338 | 1688336 | 1688337 | AACUUCGGCGGCGACAAAAUA | 1037 |
| 1688341 | 1688339 | 1688340 | UUAGAAAGACCGGGGCUUUGA | 1038 |
| 1688344 | 1688342 | 1688343 | CUUUGAUUUUCUUGGACAAAA | 1039 |
| 1688347 | 1688345 | 1688346 | UUGGACAAAUUGCCAUCUCCA | 1040 |
| 1688350 | 1688348 | 1688349 | UCUCCCCGUUUCCACAAACUA | 1041 |
| 1688353 | 1688351 | 1688352 | UCCACAAACUUGACGUGGAGA | 1042 |
| 1688356 | 1688354 | 1688355 | UCGGACUGCGUUGUUAACUUA | 1043 |
| 1688359 | 1688357 | 1688358 | AACUUUGAUUUCCUAGACGAA | 1044 |
| 1688362 | 1688360 | 1688361 | AUUUCCUAGACGAAAACGCCA | 1045 |
| 1688365 | 1688363 | 1688364 | UCUUUACCUUCGCCUCAAGUA | 1046 |
| 1688368 | 1688366 | 1688367 | ACCUUCGCCUCAAGUACUGUA | 1047 |
| 1688371 | 1688369 | 1688370 | GGCUAACUUUGAAGGGGUAAA | 1048 |
| 1688374 | 1688372 | 1688373 | AAGGGGUAAAACUGAGUAGCA | 1049 |
| 1688377 | 1688375 | 1688376 | CCGCCCUCAUUAAUCUCAAGA | 1050 |
| 1688380 | 1688378 | 1688379 | UCAAGUACUCGGUAGAAGAGA | 1051 |
| 1688383 | 1688381 | 1688382 | GUACUCGGUAGAAGAGGAGCA | 1052 |
| 1688386 | 1688384 | 1688385 | CCACACCUAGUGGACAUCAAA | 1053 |
| 1688389 | 1688387 | 1688388 | GUGGACAUCAAUCCCAGCUCA | 1054 |
| 1688392 | 1688390 | 1688391 | CUGGUCACCAAGCAGAAGAUA | 1055 |
| 1688395 | 1688393 | 1688394 | AAGAUUGACCGUGAUCUGCUA | 1056 |
| 1688398 | 1688396 | 1688397 | GAUUGACCGUGAUCUGCUGUA | 1057 |
| 1688401 | 1688399 | 1688400 | GUCAUGUCCAGCUCAAUGGAA | 1058 |
| 1688404 | 1688402 | 1688403 | AUGGAAAUCUGCGUGAUAAAA | 1059 |
| 1688407 | 1688405 | 1688406 | AUAAAGGUGGAGAUCAAGGAA | 1060 |
| 1688410 | 1688408 | 1688409 | CAAGGACCUGAACGACAAUGA | 1061 |
| 1688413 | 1688411 | 1688412 | ACGAUCCAGACUCAGGAAGCA | 1062 |
| 1688416 | 1688414 | 1688415 | GAUCCAGACUCAGGAAGCUUA | 1063 |
| 1688419 | 1688417 | 1688418 | UGCCGAACUCGUGGUGGAAAA | 1064 |
| 1688422 | 1688420 | 1688421 | CGUUGGCCUUAGUAUCAAGGA | 1065 |
| 1688425 | 1688423 | 1688424 | CAAGGUGACCGACUCCAAUGA | 1066 |
| 1688428 | 1688426 | 1688427 | CAAUGACAACAACCCGGUGUA | 1067 |
| 1688431 | 1688429 | 1688430 | GACAACAACCCGGUGUUUAGA | 1068 |
| 1688434 | 1688432 | 1688433 | GGUCUACUCCUUCUAUGGCUA | 1069 |
| 1688437 | 1688435 | 1688436 | CUUCUAUGGCUACGUCAACGA | 1070 |
| 1688440 | 1688438 | 1688439 | UGUCACUGGCGCUUUAGACUA | 1071 |
| 1688443 | 1688441 | 1688442 | UGGCGCUUUAGACUACGAAGA | 1072 |
| 1688446 | 1688444 | 1688445 | UGACAAUCCGCCGGUCAUCAA | 1073 |
| 1688449 | 1688447 | 1688448 | CAUCAACCUGCUGUCAGUCAA | 1074 |
| 1688452 | 1688450 | 1688451 | AGUCAACAGUGAGCUUGUGGA | 1075 |
| 1688455 | 1688453 | 1688454 | CCCUUUCGACUGCAGGAAUAA | 1076 |
| 1688458 | 1688456 | 1688457 | GAAUAUGAGAGCUUCUCCACA | 1077 |
| 1688461 | 1688459 | 1688460 | CUUCUCCACUAUUCUGGUGGA | 1078 |
| 1688464 | 1688462 | 1688463 | CACGACCAAUACAACCUCACA | 1079 |
| 1688467 | 1688465 | 1688466 | CAACCUCACAAUUCAGGCACA | 1080 |
| 1688470 | 1688468 | 1688469 | AGUCCUUUACCGUGCUCAUCA | 1081 |
| 1688473 | 1688471 | 1688472 | UCAUCACUGACGAAAAUGACA | 1082 |
| 1688476 | 1688474 | 1688475 | AUGACAACCACCCGCACUUUA | 1083 |
| 1688479 | 1688477 | 1688478 | UGACAACCACCCGCACUUUUA | 1084 |
| 1688482 | 1688480 | 1688481 | CUUUUCCAAGCCCUACUACCA | 1085 |
| 1688485 | 1688483 | 1688484 | CUACCAGGUCAUUGUGCAGGA | 1086 |
| 1688488 | 1688486 | 1688487 | AUUGUGCAGGAGAACAACACA | 1087 |
| 1688491 | 1688489 | 1688490 | CAUGCCUGUCUUCACCUAUGA | 1088 |
| 1688494 | 1688492 | 1688493 | CUAUGUCUCCAUCAAUCCCAA | 1089 |
| 1688497 | 1688495 | 1688496 | UCCCAACUCAGGCGACAUCUA | 1090 |
| 1688500 | 1688498 | 1688499 | CUACGCGCUGCGAUCCUUUAA | 1091 |
| 1688503 | 1688501 | 1688502 | CUUUAACCACGAGCAGACCAA | 1092 |
| 1688506 | 1688504 | 1688505 | GACCAAGGCGUUCGAAUUCAA | 1093 |
| 1688509 | 1688507 | 1688508 | CGAAUUCAAGGUGCUGGCCAA | 1094 |
| 1688512 | 1688510 | 1688511 | CAUCCUCGACGUCAACGACAA | 1095 |
| 1688515 | 1688513 | 1688514 | CACAGCCCCACCUCUGAUUAA | 1096 |
| 1688518 | 1688516 | 1688517 | AGGCUACCUGGUGACUGUUGA | 1097 |
| 1688521 | 1688519 | 1688520 | UGUUGUCAAGGCAGAAGACUA | 1098 |
| 1688524 | 1688522 | 1688523 | AGACUACGAUGAGGGCGAAAA | 1099 |
| 1688527 | 1688525 | 1688526 | ACUACGAUGAGGGCGAAAAUA | 1100 |
| 1688530 | 1688528 | 1688529 | CCGCGGCUUCUUUGAAAUAGA | 1101 |
| 1688533 | 1688531 | 1688532 | AAUAGACCAGGUCAAUGGCGA | 1102 |
| 1688536 | 1688534 | 1688535 | AGACCAGGUCAAUGGCGAAGA | 1103 |
| 1688539 | 1688537 | 1688538 | CCAAGUCCUCCUAUGAGCUUA | 1104 |
| 1688542 | 1688540 | 1688541 | UGAGCUUAUCGUGGUGGCUCA | 1105 |
| 1688545 | 1688543 | 1688544 | CUGCUCUCGUCCUAAUCUACA | 1106 |
| 1688548 | 1688546 | 1688547 | AUCUACUUGUCCCCUGCUCUA | 1107 |
| 1688551 | 1688549 | 1688550 | AGAGUCAAUGGGCUCUGUGAA | 1108 |
| 1688554 | 1688552 | 1688553 | UGUGAACUUGUCCUUGAUUUA | 1109 |
| 1688557 | 1688555 | 1688556 | GAUUUUCAUUAUUGCCCUGGA | 1110 |
| 1688560 | 1688558 | 1688559 | AUUAUUGCCCUGGGCUCCAUA | 1111 |
| 1688563 | 1688561 | 1688562 | UUGCGGGCAUCCUCUUUGUAA | 1112 |
| 1688566 | 1688564 | 1688565 | UUGUAACUAUGAUCUUCGUGA | 1113 |
| 1688569 | 1688567 | 1688568 | UUCGUGGCAAUCAAGUGCAAA | 1114 |
| 1688572 | 1688570 | 1688571 | UGCAAGCGAGACAACAAAGAA | 1115 |
| 1688575 | 1688573 | 1688574 | AAAGAGAUCCGGACCUACAAA | 1116 |
| 1688578 | 1688576 | 1688577 | UACAACUGCAGUAAUUGUUUA | 1117 |
| 1688581 | 1688579 | 1688580 | UGUUUAACCAUCACUUGUCUA | 1118 |
| 1688584 | 1688582 | 1688583 | UGUCUCCUCGGCUGUUUUAUA | 1119 |
| 1688587 | 1688585 | 1688586 | UUUAUAAAAGGACAAAACAGA | 1120 |
| 1688590 | 1688588 | 1688589 | AACAGCAAGUGUCUGCAUUGA | 1121 |
| 1688593 | 1688591 | 1688592 | UGCAUUGCAUCUCGGUUUCUA | 1122 |
| 1688596 | 1688594 | 1688595 | CUCGGUUUCUCCCAUUAGCGA | 1123 |
| 1688599 | 1688597 | 1688598 | UAGCGAGGAGCAAGACAAAAA | 1124 |
| 1688602 | 1688600 | 1688601 | CAAAAAGACAGAGGAGAAAGA | 1125 |
| 1688605 | 1688603 | 1688604 | GAAAGUGAGCCUAAGGGGAAA | 1126 |
| 1688608 | 1688606 | 1688607 | GGGAAAGAGAAUUGCUGAGUA | 1127 |
| 1688611 | 1688609 | 1688610 | GCUGAGUACUCCUAUGGGCAA | 1128 |
| 1688614 | 1688612 | 1688613 | GGGCAUCAAAAGAAAUCAAGA | 1129 |
| 1688617 | 1688615 | 1688616 | UCAAGCAAGAAGAAAAAAAUA | 1130 |
| 1688620 | 1688618 | 1688619 | AAAAUCAGUAAGAAUGACAUA | 1131 |
| 1688623 | 1688621 | 1688622 | AGAAUGACAUCCGCCUGGUAA | 1132 |
| 1688626 | 1688624 | 1688625 | UGUGGAGGAGACAGACAAGAA | 1133 |
| 1688946 | 1688944 | 1688945 | CAAGAUGAACGUUGUCAGUUA | 1134 |
| 1688949 | 1688947 | 1688948 | CGUUGUCAGUUGCUCUUCCCA | 1135 |
| 1688952 | 1688950 | 1688951 | GACCUCCUCCCUCAACUAUUA | 1136 |
| 1688955 | 1688953 | 1688954 | CUAUUUUGACUACCACCAGCA | 1137 |
| 1688958 | 1688956 | 1688957 | UUUGACUACCACCAGCAGACA | 1138 |
| 1688961 | 1688959 | 1688960 | UCUGAGAGCACUUUCCUGAAA | 1139 |
| 1688964 | 1688962 | 1688963 | CUGAAUGUGGAGAACCAGAAA | 1140 |
| 1688967 | 1688965 | 1688966 | UGUGGAGAACCAGAAUACCCA | 1141 |
| 1688970 | 1688968 | 1688969 | UACCCGCAACACCAGUGCUAA | 1142 |
| 1688973 | 1688971 | 1688972 | UGCUAACCACAUCUACCAUCA | 1143 |
| 1688976 | 1688974 | 1688975 | CCAUCACUCUUUCAACAGCCA | 1144 |
| 1688979 | 1688977 | 1688978 | CAUCACUCUUUCAACAGCCAA | 1145 |
| 1688982 | 1688980 | 1688981 | GCAGCCUGACCUGAUUAUCAA | 1146 |
| 1688985 | 1688983 | 1688984 | AUUAUCAACGGUGUGCCUCUA | 1147 |
| 1688988 | 1688986 | 1688987 | UCUGCCUGAGACUGAAAACUA | 1148 |
| 1688991 | 1688989 | 1688990 | AAACUAUUCUUUUGACUCCAA | 1149 |
| 1688994 | 1688992 | 1688993 | ACUCCAACUACGUGAAUAGCA | 1150 |
| 1688997 | 1688995 | 1688996 | AUAGCCGAGCCCAUUUAAUCA | 1151 |
| 1689000 | 1688998 | 1688999 | UAAUCAAGAGCAGCUCCACCA | 1152 |
| 1689003 | 1689001 | 1689002 | CUCCACCUUCAAGGACUUAGA | 1153 |
| 1689006 | 1689004 | 1689005 | CAAGGACUUAGAGGGCAACAA | 1154 |
| 1689009 | 1689007 | 1689008 | CAGCCUGAAGGAUAGUGGACA | 1155 |
| 1689012 | 1689010 | 1689011 | AGUGGACAUGAGGAGAGUGAA | 1156 |
| 1689015 | 1689013 | 1689014 | AGUGACCAAACUGACAGUGAA | 1157 |
| 1689018 | 1689016 | 1689017 | UGACAGUGAGCAUGAUGUCCA | 1158 |
| 1689021 | 1689019 | 1689020 | CGGAGCCUGUAUUGUGAUACA | 1159 |
| 1689024 | 1689022 | 1689023 | GAUACUGCUGUCAACGAUGUA | 1160 |
| 1689027 | 1689025 | 1689026 | CGAUGUGCUGAACACCAGUGA | 1161 |
| 1689030 | 1689028 | 1689029 | GGGAUCUCAGAUGCCUGAUCA | 1162 |
| 1689033 | 1689031 | 1689032 | UGAUCAUGAUCAGAAUGAAGA | 1163 |
| 1689036 | 1689034 | 1689035 | UGAAGGAUUUCAUUGCCGGGA | 1164 |
| 1689039 | 1689037 | 1689038 | UUCAUUGCCGGGAAGAAUGCA | 1165 |
| 1689042 | 1689040 | 1689041 | UGAGCAUGUGAGGAACAUCAA | 1166 |
| 1689045 | 1689043 | 1689044 | CAUCAUCGCGCUGUCUAUUGA | 1167 |
| 1689048 | 1689046 | 1689047 | UAUUGAAGCUACUGCUGCUGA | 1168 |
| 1689051 | 1689049 | 1689050 | GAAGCUACUGCUGCUGAUGUA | 1169 |
| 1689054 | 1689052 | 1689053 | AUGUCGAGGCUUAUGACGACA | 1170 |
| 1689057 | 1689055 | 1689056 | CAAACGGACUUUCGCAACCUA | 1171 |
| 1689060 | 1689058 | 1689059 | AACCUUUGGGAAAGAUGUCAA | 1172 |
| 1689063 | 1689061 | 1689062 | UGGGAAAGAUGUCAGCGACCA | 1173 |
| 1689066 | 1689064 | 1689065 | AAAGGCAAGAGGACUGUCGAA | 1174 |
| 1689069 | 1689067 | 1689068 | GAGGACUGUCGAUGUGACCAA | 1175 |
| 1689072 | 1689070 | 1689071 | GCCUUCCGUGUCUUACACCAA | 1176 |
| 1689075 | 1689073 | 1689074 | CCUUCCGUGUCUUACACCAUA | 1177 |
| 1689078 | 1689076 | 1689077 | UGAUCUGGAGCAGUAUGUCAA | 1178 |
| 1689081 | 1689079 | 1689080 | UGUCAACAAUGUCAACAAUGA | 1179 |
| 1689084 | 1689082 | 1689083 | GUCAACAAUGGCCCUACUCGA | 1180 |
| 1689087 | 1689085 | 1689086 | GAGCUGAUAGCGAGAAAGUCA | 1181 |
| 1689090 | 1689088 | 1689089 | AAAGUCAUGCAUGAGGUCAGA | 1182 |
| 1689093 | 1689091 | 1689092 | UUCUGAAGGAAGGUCGCAACA | 1183 |
| 1689096 | 1689094 | 1689095 | GGAAGGUCGCAACAAAGAGUA | 1184 |
| 1689099 | 1689097 | 1689098 | CAAAGAGUCCCCUGGUGUGAA | 1185 |
| 1689102 | 1689100 | 1689101 | UGUGAAGCGUCUGAAGGAUAA | 1186 |
| 1689105 | 1689103 | 1689104 | GGAUAUCGUUCUCUAAACCAA | 1187 |
| 1689108 | 1689106 | 1689107 | AACCAGUCUCCAGGAAGAAGA | 1188 |
| 1689111 | 1689109 | 1689110 | AGAAGAGAAAGAAACCACACA | 1189 |
| 1689114 | 1689112 | 1689113 | CCACACUGGCUAGUGAAGAAA | 1190 |
| 1689117 | 1689115 | 1689116 | AAGAAGCAGGAGCUUCUUGUA | 1191 |
| 1689120 | 1689118 | 1689119 | CUUGUUUUAAUUGCUCACCAA | 1192 |
| 1689123 | 1689121 | 1689122 | CACCAAUGGUUGGUUCUUGAA | 1193 |
| 1689126 | 1689124 | 1689125 | CUUGAGUGGCUAUAUUUCAGA | 1194 |
| 1689129 | 1689127 | 1689128 | UUCAGAGCUUUUCCUAAAUGA | 1195 |
| 1689132 | 1689130 | 1689131 | AAAUGUAUUGUUUAUAGGUGA | 1196 |
| 1689135 | 1689133 | 1689134 | AGGUGAUUAUCAUUCUGUGAA | 1197 |
| 1689138 | 1689136 | 1689137 | UGUGACAGUCCCUUGUUUCAA | 1198 |
| 1689141 | 1689139 | 1689140 | AGUCCCUUGUUUCAACAGGCA | 1199 |
| 1689144 | 1689142 | 1689143 | GGGUGUUCAGUUGGAGCAAAA | 1200 |
| 1689147 | 1689145 | 1689146 | GCAAAUUAGCUUUGGCUUGAA | 1201 |
| 1689150 | 1689148 | 1689149 | UGGCUUGAGUUGUUCAUGGGA | 1202 |
| 1689153 | 1689151 | 1689152 | GUUCAUGGGGCCUUGAUGUUA | 1203 |
| 1689156 | 1689154 | 1689155 | AUGUUGGGGAAACAGAGACAA | 1204 |
| 1689159 | 1689157 | 1689158 | AGACAAAUUCAGUUGUGAAAA | 1205 |
| 1689162 | 1689160 | 1689161 | UGAAAAGUAUUAUGUAUUAAA | 1206 |
| 1689165 | 1689163 | 1689164 | AUUAAGUGUUUGAAUUUAUAA | 1207 |
| 1689168 | 1689166 | 1689167 | UUAUAUAUUUUUCUAUGUCAA | 1208 |
| 1689171 | 1689169 | 1689170 | AUGUCAAAAUUAUAAUAUAAA | 1209 |
| 1689174 | 1689172 | 1689173 | UAUAAAUUACCAUUGUUUGUA | 1210 |
| 1689177 | 1689175 | 1689176 | UUUGUGGAGGAUUACAUUUAA | 1211 |
| 1689180 | 1689178 | 1689179 | AUUUAAAAAAGCAAAAAGUGA | 1212 |
| 1689183 | 1689181 | 1689182 | AAGUGAAAAAAAAAAGCUCUA | 1213 |
| 1689186 | 1689184 | 1689185 | GCUCUGGACACCUUUAAUAAA | 1214 |
| 1689189 | 1689187 | 1689188 | AAUAAGCUCGCCACUGUUUUA | 1215 |
| 1689192 | 1689190 | 1689191 | GUUUUUUGUAGUGUAGACAAA | 1216 |
| 1689195 | 1689193 | 1689194 | GACAAGUUAAUGGUCAUUUAA | 1217 |
| 1689198 | 1689196 | 1689197 | AUUUAUUGUGUACUAUUCAUA | 1218 |
| 1689201 | 1689199 | 1689200 | UUCAUUGAUUCAGUGAUGUGA | 1219 |
| 1689204 | 1689202 | 1689203 | AUGUGAAAUUGAGCCCCCAAA | 1220 |
| 1689207 | 1689205 | 1689206 | CCCAAAAGGUUGUUUCUGAAA | 1221 |
| 1689210 | 1689208 | 1689209 | CUGAAGCUCGAGUACUUCCCA | 1222 |
| 1689213 | 1689211 | 1689212 | CUUCCCAAUGCCGCUACUUUA | 1223 |
| 1689216 | 1689214 | 1689215 | GUGGACACCCCUGCUUUAAAA | 1224 |
| 1689219 | 1689217 | 1689218 | UUAAAAACCCUUUUGCUAGCA | 1225 |
| 1689222 | 1689220 | 1689221 | CUAGCUGUGCUAUUGUUGUAA | 1226 |
| 1689225 | 1689223 | 1689224 | UUGUAUUUGAUAUUGCAAAUA | 1227 |
| 1689228 | 1689226 | 1689227 | CAAAUUGCUAUGUGUGUGGUA | 1228 |
| 1689231 | 1689229 | 1689230 | GUGGUGAUGGCAAAAGGCUUA | 1229 |
| 1689234 | 1689232 | 1689233 | GGCUUUUAAAAGUUGGUGUUA | 1230 |
| 1689237 | 1689235 | 1689236 | GUGUUUUCUUUUUCUUAAAAA | 1231 |
| 1689240 | 1689238 | 1689239 | UAAAAAAAUAAUAAAACUACA | 1232 |
| 1689243 | 1689241 | 1689242 | ACUACAGACAAAAACAAAGUA | 1233 |
| 1689246 | 1689244 | 1689245 | AAAGUGCAAACAACUGAGACA | 1234 |
| 1689249 | 1689247 | 1689248 | GAGACAGCAAAUGAAUGAGCA | 1235 |
| 1689252 | 1689250 | 1689251 | UGAGCAACACCAUGCAUAUAA | 1236 |
| 1689255 | 1689253 | 1689254 | AUAUAGAUUGAGUACUUGGAA | 1237 |
| 1689258 | 1689256 | 1689257 | UUGGAGAAUACUCACGUUUUA | 1238 |
| 1689261 | 1689259 | 1689260 | GUUUUUUAACUCAUGUAGUGA | 1239 |
| 1689264 | 1689262 | 1689263 | UAGUGAUUGCUCACCACUUUA | 1240 |
| 1689267 | 1689265 | 1689266 | ACUUUGCAAAGUAUUUUUCCA | 1241 |
| 1689270 | 1689268 | 1689269 | UUUCCUGCCUUUCAGUGGUCA | 1242 |
| 1689273 | 1689271 | 1689272 | UUCCUGCCUUUCAGUGGUCUA | 1243 |
| 1689276 | 1689274 | 1689275 | CCCAGGUCCAUGAUAGAUCAA | 1244 |
| 1689279 | 1689277 | 1689278 | GAUCAUUGCAGAAAGUCAUUA | 1245 |
| 1689282 | 1689280 | 1689281 | UCAUUUUUGGAUAGGCUGCUA | 1246 |
| 1689285 | 1689283 | 1689284 | CUGCUAUCUAAUUUGCAGUUA | 1247 |
| 1689288 | 1689286 | 1689287 | CAGUUGUCAGAAAUACUGUGA | 1248 |
| 1689291 | 1689289 | 1689290 | CUGUGCUGAAAGUUUUAUGAA | 1249 |
| 1689294 | 1689292 | 1689293 | UAUGACAUCCAUCUUUUAAAA | 1250 |
| 1689297 | 1689295 | 1689296 | UUAAAUUUUCAGGCCCUGAAA | 1251 |
| 1689300 | 1689298 | 1689299 | CAGGCCCUGAACAGGAAAGUA | 1252 |
| 1689303 | 1689301 | 1689302 | AAAGUUGCUCCUGAAGUUAUA | 1253 |
| 1689306 | 1689304 | 1689305 | GUUAUGUUUCCAGGCUUAGAA | 1254 |
| 1689309 | 1689307 | 1689308 | UUAGAAAUUCCCUCUCUCCAA | 1255 |
| 1689312 | 1689310 | 1689311 | CUCCAAUCAUCUAAAAUUGAA | 1256 |
| 1689315 | 1689313 | 1689314 | AUUGAAGAUGACUGAAUCUAA | 1257 |
| 1689318 | 1689316 | 1689317 | AUCUAUUUUAAGAUCUAAAUA | 1258 |
| 1689321 | 1689319 | 1689320 | UAAAUUAGCAUCUCUUCAGAA | 1259 |
| 1689324 | 1689322 | 1689323 | UCAGACACACACUUCCUGAUA | 1260 |
| 1689327 | 1689325 | 1689326 | CUGAUUCAGUGUUUCCCAAUA | 1261 |
| 1689330 | 1689328 | 1689329 | CCAAUCAUGAUUAGAAUUAGA | 1262 |
| 1689333 | 1689331 | 1689332 | AUUAGACUGCAAAGCACGUCA | 1263 |
| 1689336 | 1689334 | 1689335 | AGACUGCAAAGCACGUCAUGA | 1264 |
| 1689339 | 1689337 | 1689338 | UGGGAUUGAGAACUCCAUGUA | 1265 |
| 1689342 | 1689340 | 1689341 | CAUGUUGCUCUGUAUCUUAGA | 1266 |
| 1689345 | 1689343 | 1689344 | CUUAGGCUUAUAUACGUAGGA | 1267 |
| 1689348 | 1689346 | 1689347 | GUAGGGAUAGAGUAAGCAGUA | 1268 |
| 1689351 | 1689349 | 1689350 | GCAGUACAAAGUGUAUAUUUA | 1269 |
| 1689354 | 1689352 | 1689353 | UAUUUUGGAAAAGACAGGUAA | 1270 |
| 1689357 | 1689355 | 1689356 | AGGUAACAGGUAACAGCAUCA | 1271 |
| 1689360 | 1689358 | 1689359 | GCAUCGCUAAUCCAAUUACUA | 1272 |
| 1689363 | 1689361 | 1689362 | UUACUGUGCCUUCUAAACGGA | 1273 |
| 1689366 | 1689364 | 1689365 | AACGGAGACACUCAGACACUA | 1274 |
| 1689369 | 1689367 | 1689368 | ACACUUGAACUCAUCUUUUUA | 1275 |
| 1689372 | 1689370 | 1689371 | UUUUUAUGACUAAUAGUUUUA | 1276 |
| 1689375 | 1689373 | 1689374 | GUUUUUUGAUUAAAAAUGUGA | 1277 |
| 1689378 | 1689376 | 1689377 | AUGUGAACAAGAAAAUACUAA | 1278 |
| 1689381 | 1689379 | 1689380 | UACUAAAAUAAAAAUCCUUUA | 1279 |
| 1689384 | 1689382 | 1689383 | CCUUUCGAUUUAGCCAACUCA | 1280 |
| 1689387 | 1689385 | 1689386 | AACUCUUUUUGCUGUAGGCUA | 1281 |
| 1689390 | 1689388 | 1689389 | AGGCUAGGAAUACACCCUUUA | 1282 |
| 1689393 | 1689391 | 1689392 | CCUUUUUAAAUUAACACACUA | 1283 |
| 1689396 | 1689394 | 1689395 | ACACUGUAGAUCCUUUUUUUA | 1284 |
| 1689399 | 1689397 | 1689398 | UUUUUCUGUUUUAACAUUCCA | 1285 |
| 1689402 | 1689400 | 1689401 | AUUCCUCUAACUCCCCCAUUA | 1286 |
| 1689405 | 1689403 | 1689404 | CCAUUUAUCUUUUGUCUAUUA | 1287 |
| 1689408 | 1689406 | 1689407 | CUAUUAAUAUUCACUAGCCAA | 1288 |
| 1689411 | 1689409 | 1689410 | AGCCAACAUAGUAUUUUUGCA | 1289 |
| 1689414 | 1689412 | 1689413 | UUUGCAGCCUAAGAGUUCAUA | 1290 |
| 1689417 | 1689415 | 1689416 | UUCAUUAUUUAAAAAUAAACA | 1291 |
| 1689420 | 1689418 | 1689419 | UAAACUAAAGAAAUAUGUCAA | 1292 |
| 1689423 | 1689421 | 1689422 | UGUCACCUUUGUUCUGCCUUA | 1293 |
| 1689426 | 1689424 | 1689425 | GCCUUGGUCUUAAGAGAGUUA | 1294 |
| 1689429 | 1689427 | 1689428 | GAGUUGUUUCUAGAGAAACAA | 1295 |
| 1689432 | 1689430 | 1689431 | AAACAAGUUUUAUGGUUCUGA | 1296 |
| 1689435 | 1689433 | 1689434 | UUCUGUUUUCAUUUGUUUCAA | 1297 |
| 1689438 | 1689436 | 1689437 | UUUCAUUUUUUGAAAUUCAGA | 1298 |
| 1689441 | 1689439 | 1689440 | UUCAGGAAUACACAGAGAGAA | 1299 |
| 1689444 | 1689442 | 1689443 | AGAGAAGGCCUAGAGGUUAGA | 1300 |
| 1689447 | 1689445 | 1689446 | GUUAGAGCACUAGAAUGCAAA | 1301 |
| 1689450 | 1689448 | 1689449 | UGCAAAAGAGGAUUUAUUUUA | 1302 |
| 1689453 | 1689451 | 1689452 | AUUUUCUUAAGUUUAGGUAGA | 1303 |
| 1689456 | 1689454 | 1689455 | GGUAGAUAAGUCAGCUCUUUA | 1304 |
| 1689459 | 1689457 | 1689458 | UCUUUUGAAUGUUUUACUUAA | 1305 |
| 1689462 | 1689460 | 1689461 | ACUUAUUUUUGCCUGAGUUCA | 1306 |
| 1689465 | 1689463 | 1689464 | AGUUCCUAGCUUUACGCAUUA | 1307 |
| 1689468 | 1689466 | 1689467 | GCAUUACUAGGAAUAUUUUCA | 1308 |
| 1689471 | 1689469 | 1689470 | UUUUCUUUUACAGGAGGGAGA | 1309 |
| 1689474 | 1689472 | 1689473 | CAGGAGGGAGAAGGGUUUUGA | 1310 |
| 1689477 | 1689475 | 1689476 | GUUAGGUAGGGGAGAACAUUA | 1311 |
| 1689480 | 1689478 | 1689479 | ACAUUUCUGAAAAAGAACUUA | 1312 |
| 1689483 | 1689481 | 1689482 | AACUUAUAAUGAAGCUACAAA | 1313 |
| 1689486 | 1689484 | 1689485 | UACAAAUCCAUCCUUAUUUCA | 1314 |
| 1689489 | 1689487 | 1689488 | AUUUCUUAUUCAGUGCUGAAA | 1315 |
| 1689492 | 1689490 | 1689491 | CUGAAUACUACCUCAUGCAAA | 1316 |
| 1689495 | 1689493 | 1689494 | UGCAAAAUAUUGGUGUGGCUA | 1317 |
| 1689498 | 1689496 | 1689497 | UGGCUGCAAAUUUCCCUCUGA | 1318 |
| 1689501 | 1689499 | 1689500 | CUCUGAAGCUGACACAAUUAA | 1319 |
| 1689504 | 1689502 | 1689503 | AAUUAAGGAUGAAUUACCAUA | 1320 |
| 1689507 | 1689505 | 1689506 | ACCAUAACAUCUUCAUUUUUA | 1321 |
| 1689510 | 1689508 | 1689509 | UUUUUCCUCAUUUCAUUGCCA | 1322 |
| 1689513 | 1689511 | 1689512 | UUGCCUCUCAUACAGUUUUCA | 1323 |
| 1689516 | 1689514 | 1689515 | UUUUCUCCCUCUGAUUUAUUA | 1324 |
| 1689519 | 1689517 | 1689518 | UUAUUUCAUUUUGGUAGUGGA | 1325 |
| 1689522 | 1689520 | 1689521 | AGUGGAUUUUGAAUUAAGUAA | 1326 |
| 1689525 | 1689523 | 1689524 | AAGUAUUUAUUUCUCUUUGCA | 1327 |
| 1689528 | 1689526 | 1689527 | UUUGCAAGUGACUAUUUGAUA | 1328 |
| 1689531 | 1689529 | 1689530 | UUGAUUAACACAUUAAAAAUA | 1329 |
| 1689534 | 1689532 | 1689533 | AAAAUUUUUUAAAAAUUUCCA | 1330 |
| 1689537 | 1689535 | 1689536 | UUUCCCCUUUAAGUUAUGAUA | 1331 |
| 1689540 | 1689538 | 1689539 | AUGAUGGCUGCCUAUAGAAUA | 1332 |
| 1689543 | 1689541 | 1689542 | AGAAUUUAGACUGUUUCUCAA | 1333 |
| 1689546 | 1689544 | 1689545 | UCUCACCCUGAUCCAUCCUGA | 1334 |
| 1689549 | 1689547 | 1689548 | UCCUGAUAUUAUGUUAUUAGA | 1335 |
| 1689552 | 1689550 | 1689551 | AUUAGCUACGAUUUCAAGGUA | 1336 |
| 1689555 | 1689553 | 1689554 | AAGGUCACUUUGAAGUCAGAA | 1337 |
| 1689558 | 1689556 | 1689557 | UCAGACUUCACAGUUUUCUAA | 1338 |
| 1689561 | 1689559 | 1689560 | UUCUACAGGUGUAUUUCUUGA | 1339 |
| 1689564 | 1689562 | 1689563 | UCUUGCUAUGCUCAGUGGGAA | 1340 |
| 1689567 | 1689565 | 1689566 | CUUGCUAUGCUCAGUGGGAAA | 1341 |
| 1689570 | 1689568 | 1689569 | AACCAGGGAGCAGAAAAGAUA | 1342 |
| 1689573 | 1689571 | 1689572 | AAGAUUGUGGAAAGUGGGAAA | 1343 |
| 1689576 | 1689574 | 1689575 | GGGAACAUUAAUUGUCACGUA | 1344 |
| 1689579 | 1689577 | 1689578 | AUUGUCACGUGGCUGUUGAGA | 1345 |
| 1689582 | 1689580 | 1689581 | UGCCUCUUACCAGCCAAGUUA | 1346 |
| 1689585 | 1689583 | 1689584 | AAGUUUUAAAUGGUUAGAGUA | 1347 |
| 1689588 | 1689586 | 1689587 | AGAGUGGCAUUGGCAUUGAUA | 1348 |
| 1689591 | 1689589 | 1689590 | UUGAUUUUGCUUUUCUCUUGA | 1349 |
| 1689594 | 1689592 | 1689593 | UCUUGGGUUUUCUUCAUUUCA | 1350 |
| 1689597 | 1689595 | 1689596 | AUUUCUUUUCAUAAUUCAUCA | 1351 |
| 1689600 | 1689598 | 1689599 | UCAUCUUCAAGCUAUAGAUCA | 1352 |
| 1689603 | 1689601 | 1689602 | AGAUCCAUCCUUCUUCCCUAA | 1353 |
| 1689606 | 1689604 | 1689605 | CCCUAUCCAAAUACUUUGUUA | 1354 |
| 1689609 | 1689607 | 1689608 | UUUGUUCUCAGUGGGCAGUCA | 1355 |
| 1689612 | 1689610 | 1689611 | CAGUCAGAUUUUUUUGGCUUA | 1356 |
| 1689615 | 1689613 | 1689614 | UGGCUUCCAAACCUCACUGAA | 1357 |
| 1689618 | 1689616 | 1689617 | ACUGAGCAUGUUCAGAGAAUA | 1358 |
| 1689621 | 1689619 | 1689620 | AGAAUUCCUCUCAUUAAGGAA | 1359 |
| 1689624 | 1689622 | 1689623 | AAGGAAAUAUUUUUCCCCACA | 1360 |
| 1689627 | 1689625 | 1689626 | CCCACAAUUGUCUGAUGUAUA | 1361 |
| 1689630 | 1689628 | 1689629 | UGUAUGAGUCCUCUCAGAAGA | 1362 |
| 1689633 | 1689631 | 1689632 | AGAAGCACAAGUGCAUACUAA | 1363 |
| 1689636 | 1689634 | 1689635 | UACUAGGAAGGCUUUGUAAAA | 1364 |
| 1689639 | 1689637 | 1689638 | GUAAACUGGUUUUGUAGGCAA | 1365 |
| 1689642 | 1689640 | 1689641 | UGGUUUUGUAGGCAGGCUGUA | 1366 |
| 1689645 | 1689643 | 1689644 | GCUGUACCUGCAAAUAGGCAA | 1367 |
| 1689648 | 1689646 | 1689647 | AGGCAUUCUGAGCAAGGAUCA | 1368 |
| 1689651 | 1689649 | 1689650 | GGAUCCUAAAUUUCCCUUUGA | 1369 |
| 1689654 | 1689652 | 1689653 | UUUCCCUUUGGCUCAUGUCCA | 1370 |
| 1689657 | 1689655 | 1689656 | ACCUUCUCCUUUUUGGCAUGA | 1371 |
| 1689660 | 1689658 | 1689659 | GCAUGUCUGGUGAAAAGCUAA | 1372 |
| 1689663 | 1689661 | 1689662 | AAGCUACAAGGUUCACAGGGA | 1373 |
| 1689666 | 1689664 | 1689665 | CAGGGCCAAAUAUUCUCCCUA | 1374 |
| 1689669 | 1689667 | 1689668 | UCCCUUUUGUUCUCUAAACAA | 1375 |
| 1689672 | 1689670 | 1689671 | AAACAUGUUGCACACAUAAAA | 1376 |
| 1689675 | 1689673 | 1689674 | AUAAACUCAAGGGAGUGAUUA | 1377 |
| 1689678 | 1689676 | 1689677 | UGAUUUCGGUUCCUAAGUCCA | 1378 |
| 1689681 | 1689679 | 1689680 | AGUCCUAGGGAGUUCUAUUCA | 1379 |
| 1689684 | 1689682 | 1689683 | UAUUCCAUUUAUUCUGAUGUA | 1380 |
| 1689687 | 1689685 | 1689686 | GAUGUCCCUUUUAAGGAGGAA | 1381 |
| 1689690 | 1689688 | 1689689 | GAGGAGUUGGUGUUGAAGUGA | 1382 |
| 1689693 | 1689691 | 1689692 | AAGUGUGACAUGCUUUAAUGA | 1383 |
| 1689696 | 1689694 | 1689695 | UAAUGAUAACUGCUUUGGCAA | 1384 |
| 1689699 | 1689697 | 1689698 | UGGCAAUUCCCCAGACAGGUA | 1385 |
| 1689702 | 1689700 | 1689701 | CAGGUAUACUUCUUCAUCCAA | 1386 |
| 1689705 | 1689703 | 1689704 | AUCCAAAAUUGCACCUAAGGA | 1387 |
| 1689708 | 1689706 | 1689707 | UAAGGUGUUCAUUUAGACAAA | 1388 |
| 1689711 | 1689709 | 1689710 | GACAAAAUUGUUCGCUUAGUA | 1389 |
| 1689714 | 1689712 | 1689713 | UUAGUGAUCCACUGAUCUCUA | 1390 |
| 1689717 | 1689715 | 1689716 | UCUCUAAUCAGAAAUAUUUUA | 1391 |
| 1689720 | 1689718 | 1689719 | GAAAUAUUUUUAAAAAAUACA | 1392 |
| 1689723 | 1689721 | 1689722 | AAUACAAUUUAGAUCUCUGAA | 1393 |
| 1689726 | 1689724 | 1689725 | UCUGAAAAUUGGAACCACUCA | 1394 |
| 1689729 | 1689727 | 1689728 | CACUCAAGAGUAUCAUCUUGA | 1395 |
| 1689732 | 1689730 | 1689731 | UCUUGAUAGUGUUACUUCAGA | 1396 |
| 1689735 | 1689733 | 1689734 | UUCAGCAUAUGACUGACUGAA | 1397 |
| 1689738 | 1689736 | 1689737 | ACUGAUUUUAUGAAGGGAGUA | 1398 |
| 1689741 | 1689739 | 1689740 | GGAGUUUAGCUGACUUUUCCA | 1399 |
| 1689744 | 1689742 | 1689743 | UUUCCCUGUAGCCCUAUCAUA | 1400 |
| 1689747 | 1689745 | 1689746 | AUCAUAUUAAAAACAAAAAGA | 1401 |
| 1689750 | 1689748 | 1689749 | AAAAGUGGUCCACUUGAAGCA | 1402 |
| 1689753 | 1689751 | 1689752 | UUGAAGCAGCAAGGACAAAGA | 1403 |
| 1689756 | 1689754 | 1689755 | GGACAAAGGGCAGUUUAGGGA | 1404 |
| 1689759 | 1689757 | 1689758 | UUGGGCAUCCAGAGUGGAAAA | 1405 |
| 1689762 | 1689760 | 1689761 | GGAAAAGGAUUAUUAUUGUAA | 1406 |
| 1689765 | 1689763 | 1689764 | UUGUACAUAAUAGCAGUUCUA | 1407 |
| 1689768 | 1689766 | 1689767 | GUUCUGGCUGGAAUUCCAAUA | 1408 |
| 1689771 | 1689769 | 1689770 | CCAAUCCACUUUCUGGGGAUA | 1409 |
| 1689774 | 1689772 | 1689773 | GGGAUCAUUUUGAUGUUGAAA | 1410 |
| 1689777 | 1689775 | 1689776 | UUGAAUGUUCCCCCAAAGCAA | 1411 |
| 1689780 | 1689778 | 1689779 | AAGCAUUCUGACAAGUUUGGA | 1412 |
| 1689783 | 1689781 | 1689782 | UUUGGUCCACUUGGCUCUUUA | 1413 |
| 1689786 | 1689784 | 1689785 | UCUUUACCCCGGGUAAUAAGA | 1414 |
| 1689789 | 1689787 | 1689788 | AUAAGACCCUUAUUGACUUUA | 1415 |
| 1689792 | 1689790 | 1689791 | ACUUUGAAAAACUGGCAUAAA | 1416 |
| 1689795 | 1689793 | 1689794 | CAUAAAUGAGCUUAAGAAAUA | 1417 |
| 1689798 | 1689796 | 1689797 | GAAAUCCAUUGUAUACAGUCA | 1418 |
| 1689801 | 1689799 | 1689800 | CAGUCCUAGUGUUUGCCUUCA | 1419 |
| 1689804 | 1689802 | 1689803 | CCUUCUUGGUAAAAUUACAUA | 1420 |
| 1689807 | 1689805 | 1689806 | UACAUUUAAGGAUGUCAUUUA | 1421 |
| 1689810 | 1689808 | 1689809 | CAUUUCAUGAGUCAAAUUGUA | 1422 |
| 1689813 | 1689811 | 1689812 | AUUGUAUCUGCUGACACUGAA | 1423 |
| 1689816 | 1689814 | 1689815 | ACUGAAGUGUAUUGAGAUGUA | 1424 |
| 1689819 | 1689817 | 1689818 | GAUGUUGUUCUUUAGUUUAAA | 1425 |
| 1689822 | 1689820 | 1689821 | UUUAAGUAUGUAACAAAGUAA | 1426 |
| 1689825 | 1689823 | 1689824 | AAGUAAUAUUCAAAGGGGAUA | 1427 |
| 1689828 | 1689826 | 1689827 | UCAAAGGGGAUGUUUGGGGGA | 1428 |
| 1689831 | 1689829 | 1689830 | ACCGGGUGGCUUUAUAUUGAA | 1429 |
| 1689834 | 1689832 | 1689833 | AUUGAUAAUGAAAUAUUCCAA | 1430 |
| 1689837 | 1689835 | 1689836 | UUCCAGAGCAAAACACAUUGA | 1431 |
| 1689840 | 1689838 | 1689839 | CAUUGUCCUAAUGAAGUUGUA | 1432 |
| 1689843 | 1689841 | 1689842 | GUUGUUGCAGCUCUCUUCGUA | 1433 |
| 1689846 | 1689844 | 1689845 | UCUUCGUUUUCUUGUGCCGCA | 1434 |
| 1689849 | 1689847 | 1689848 | UUCUUGUGCCGCUGGAAUUGA | 1435 |
| 1689852 | 1689850 | 1689851 | GAAUUGGCUUGUGGGUCACUA | 1436 |
| 1689855 | 1689853 | 1689854 | UCACUUGUUGUGCAAAAGUGA | 1437 |
| 1689858 | 1689856 | 1689857 | AAGUGAAUUAAUUUCAAAUAA | 1438 |
| 1689861 | 1689859 | 1689860 | AAAUAGGAAUCAUAUUGCUUA | 1439 |
| 1689864 | 1689862 | 1689863 | UGCUUUUGUGAAACAAAGAAA | 1440 |
| 1689867 | 1689865 | 1689866 | AAGAAAAUUUACCACCUUGUA | 1441 |
| 1689870 | 1689868 | 1689869 | CUUGUCUGAUACAUAGAUUGA | 1442 |
| 1689873 | 1689871 | 1689872 | GAUUGGAAGGCGCUCUUGGUA | 1443 |
| 1689876 | 1689874 | 1689875 | UUGGUAAAGAAUAACCCACAA | 1444 |
| 1689879 | 1689877 | 1689878 | CCACAAACACCUGAUCUUGAA | 1445 |
| 1689882 | 1689880 | 1689881 | CUUGAUUGGAAAGCAUUGUAA | 1446 |
| 1689885 | 1689883 | 1689884 | UUGUAGGUCAUUAGGGAAAAA | 1447 |
| 1689888 | 1689886 | 1689887 | GAAAACUGAUGAUGCUGACUA | 1448 |
| 1689891 | 1689889 | 1689890 | AUGAUGCUGACUGUUGCUCCA | 1449 |
| 1689894 | 1689892 | 1689893 | GCUCCAGCUGUGUUGGCAAUA | 1450 |
| 1689897 | 1689895 | 1689896 | GCAAUUCAAGAUGACCUUGUA | 1451 |
| 1689900 | 1689898 | 1689899 | CUUGUUACUAACUGAAAGUUA | 1452 |
| 1689903 | 1689901 | 1689902 | AAGUUGCUGUCAACCUUAGCA | 1453 |
| 1689906 | 1689904 | 1689905 | UUAGCAAACAAACCAGCUAAA | 1454 |
| 1689909 | 1689907 | 1689908 | GCUAACACCAUUACAGCACUA | 1455 |
| 1689912 | 1689910 | 1689911 | GCACUGGUUAAGUAGAUUUUA | 1456 |
| 1689915 | 1689913 | 1689914 | AUUUUUACUGGCACUGGCUGA | 1457 |
| 1689918 | 1689916 | 1689917 | CAGCUGAGGAAGGCUAGGAUA | 1458 |
| 1689921 | 1689919 | 1689920 | AGGAUACAGACUUUAAUGACA | 1459 |
| 1689924 | 1689922 | 1689923 | AUGACCAAGAGCCAGACUAGA | 1460 |
| 1689927 | 1689925 | 1689926 | ACUAGCUCGAGCUAGAAAUCA | 1461 |
| 1689930 | 1689928 | 1689929 | AAAUCAGCAACAAAUACCUCA | 1462 |
| 1689933 | 1689931 | 1689932 | ACCUCCUUGUGCUAUUUGAAA | 1463 |
| 1689936 | 1689934 | 1689935 | UUGAAUGCUAAGGUAUUAGUA | 1464 |
| 1689939 | 1689937 | 1689938 | UUAGUUGCAAUAGGCUUACGA | 1465 |
| 1689942 | 1689940 | 1689941 | UUGCAAUAGGCUUACGCCUUA | 1466 |
| 1689945 | 1689943 | 1689944 | GAAUGAGCCUGAUGACACUAA | 1467 |
| 1689948 | 1689946 | 1689947 | CACUAUUACUGUUUACCUGUA | 1468 |
| 1689951 | 1689949 | 1689950 | CCUGUCAAACCAGGGUGGAAA | 1469 |
| 1689954 | 1689952 | 1689953 | UGGAAAUUUAGAGGGAUGCAA | 1470 |
| 1689957 | 1689955 | 1689956 | AUGCAAGUACAGUAUGUCUCA | 1471 |
| 1689960 | 1689958 | 1689959 | GUCUCAUUAAGCAGUCUCUGA | 1472 |
| 1689963 | 1689961 | 1689962 | CUCUGUUGCUGAUUAUAUGAA | 1473 |
| 1689966 | 1689964 | 1689965 | UAUGAAGAUAAAAUUGUUGUA | 1474 |
| 1689969 | 1689967 | 1689968 | GUUGUAUAUGCUGAUCUGUAA | 1475 |
| 1689972 | 1689970 | 1689971 | CUGUAUGUUAUGUACAUUUUA | 1476 |
| 1689975 | 1689973 | 1689974 | AUUUUCACAGUGAUUGAAUCA | 1477 |
| 1689978 | 1689976 | 1689977 | GAAUCAUUGUAAAAAGAAAAA | 1478 |
| 1689981 | 1689979 | 1689980 | GAAAAAAGACAAAAAAGAAUA | 1479 |
| 1689984 | 1689982 | 1689983 | AGAAUCACCACUGUCUAUUUA | 1480 |
| 1689987 | 1689985 | 1689986 | UAUUUUAUGAAGAUGAUAAAA | 1481 |
| 1689990 | 1689988 | 1689989 | AUAAAGCAGCUUUAUUAAAUA | 1482 |
| 1689993 | 1689991 | 1689992 | UAAAUUAUUACAGUUAUGUUA | 1483 |
| 1689996 | 1689994 | 1689995 | AUGUUUCAAAUGGUGUUAUCA | 1484 |
| 1689999 | 1689997 | 1689998 | UUAUCUGCCACAUUGUUUCCA | 1485 |
| 1690002 | 1690000 | 1690001 | UUUCCUUUUAUCUGGUGGUUA | 1486 |
| 1690005 | 1690003 | 1690004 | UCUGGUGGUUCUCUGUUUCCA | 1487 |
| 1690008 | 1690006 | 1690007 | UUUCCCCCUUUAAAUGUGUUA | 1488 |
| 1690011 | 1690009 | 1690010 | GUGUUUUCUCUGCUUCAAAAA | 1489 |
| 1690014 | 1690012 | 1690013 | CAAAAAUUUAAUGCAUUUUAA | 1490 |
| 1690017 | 1690015 | 1690016 | UUUUAGUAUUAAAGGCUAUUA | 1491 |
| 1690020 | 1690018 | 1690019 | UCCUGCCUUUCAGUGGUCUGA | 1492 |
| 1690023 | 1690021 | 1690022 | UAAGGCAGUAGGGAUGGGGUA | 1493 |
| 1690026 | 1690024 | 1690025 | ACCCCACCUUCUCCUUUUUGA | 1494 |
| 1690029 | 1690027 | 1690028 | AUGUUUGGGGGUGGGGCUGGA | 1495 |
| 1690032 | 1690030 | 1690031 | CUCUAGUGAGGGGAAUGAGCA | 1496 |

### Example 7: Effect of RNAi Compounds on Human PCDH19 In Vitro, Single Dose

Double-stranded RNAi compounds described above are tested in a series of experiments for their single dose effects on PCDH19 RNA in vitro in cultured cells that express PCDH19.

Cultured cells are treated with double-stranded RNAi. RNA is isolated from the cells and PCDH19 RNA levels are measured by quantitative real-time RTPCR. A human PCDH19 primer-probe set is used to measure RNA levels. PCDH19 RNA levels are normalized to total RNA content, as measured by RIBOGREEN®.

### Example 8: Effect of Modified Oligonucleotides on Human PCDH19 RNA Levels in Neurons Differentiated from IPS Cells, Multiple Dose

Modified oligonucleotides selected from the examples above were tested at various doses in neurons differentiated from IPSCs (Gibco, Cat. #A18945). The IPSCs were differentiated into neurons using the Elixirgen Scientific Quick Neuron™ Excitatory kit (Cat. #EX-SeV-L). The neurons were aged for 2 weeks, and were treated by free uptake with various concentrations of modified oligonucleotide as specified in the tables below.

Seven days post treatment total RNA was isolated from the cells, and PCDH19 RNA levels were measured by quantitative real-time RT-PCR. Human PCDH19 primer-probe set RTS53390 (described herein above) was used to measure RNA levels as described above. PCDH19 RNA levels were normalized to total RNA content, as measured by human GAPDH. Human GAPDH was amplified using human primer probe set RTS104 (described herein above). Reduction of PCDH19 RNA is presented in the tables below as percent PCDH19 RNA, relative to the amount of PCDH19 in untreated control cells (% UTC).

The half maximal inhibitory concentration (IC₅₀) of each modified oligonucleotide was calculated using GraphPad Prism and is also presented in the tables below.

TABLE 10
Effect of modified oligonucleotides on human PCDH19
RNA levels in neurons differentiated from IPS cells

| | PCDH19 RNA (% UTC) | | | | |
|---|---|---|---|---|---|
| Compound No. | 40 nM | 200 nM | 1000 nM | 5000 nM | IC₅₀ (μM) |
| 1626339 | 82 | 63 | 33 | 16 | 0.28 |
| 1626361 | 96 | 57 | 34 | 20 | 0.18 |

TABLE 11
Effect of modified oligonucleotides on human PCDH19
RNA levels in neurons differentiated from IPS cells

| | PCDH19 RNA (% UTC) | | | | | |
|---|---|---|---|---|---|---|
| Compound | 32 | 160 | 800 | 4000 | 20000 | |
| No. | nM | nM | nM | nM | nM | IC₅₀ (μM) |
| 1653817 | 101 | 96 | 89 | 93 | 52 | 18.36 |
| 1653823 | 102 | 84 | 78 | 50 | 26 | 2.47 |
| 1653827 | 84 | 87 | 79 | 59 | 28 | 4.37 |

### Example 9: Effect of Modified Oligonucleotides on Human PCDH19 Protein Levels in Neurons Differentiated from IPS Cells, Multiple Dose

Modified oligonucleotides selected from the examples above were tested at various doses in neurons differentiated from IPSCs (neurons derived as described herein above). The neurons were aged for 2 weeks, and were treated by free uptake with various concentrations of modified oligonucleotide as specified in the table below.

Seven days post treatment, human PCDH19 protein levels in the treated neurons were determined using western blot analysis, detecting PCDH19 using a PCDH19 polyclonal antibody from Bethyl Laboratories (cat. #A304-468A). Reduction of PCDH19 protein is presented in the table below as percent PCDH19 protein relative to the amount of PCDH19 in untreated control cells (% UTC).

TABLE 12
Effect of modified oligonucleotides on human PCDH19 protein
levels in neurons differentiated from IPS cells

| | | PCDH19 Protein |
|---|---|---|
| Compound No. | Dose (nM) | (% UTC) |
| 1626339 | 5000 | 19 |
| 1626361 | 5000 | 28 |
| 1653817 | 20000 | 85 |
| 1653823 | 20000 | 52 |
| 1653827 | 20000 | 48 |

### Example 10: Effect of RNAi Compounds that Target a Human PCDH19 Nucleic Acid, In Vitro, Single Dose

RNAi compounds described in the example above were tested for their single dose effects on PCDH19 RNA in vitro. The RNAi compounds were tested in a series of experiments that had the same culture conditions.

Cultured HEK293 cells were treated with RNAi compounds at a concentration of 200 nM by Lipofectamine RNAiMAX at a density of 10,000 cells per well. After a treatment period of 72 hours, total RNA was isolated from the cells and PCDH19 RNA levels were measured by quantitative real-time RT-PCR using human primer-probe set

RTS53390 (described herein above). PCDH19 RNA levels were normalized to total RNA content, as measured by RIBOGREEN®. Reduction of PCDH19 RNA is presented in the table below as percent PCDH19 RNA relative to the amount in untreated control cells (% UTC). The values marked with a “+” indicate that the modified oligonucleotide is complementary to the amplicon region of the primer probe set. Additional assays may be used to measure the potency and efficacy of the modified oligonucleotides complementary to the amplicon region. Each table below represents a separate experiment.

TABLE 13
Reduction of PCDH19 RNA by RNAi compounds at
a concentration of 200 nM in HEK293 cells

| Compound No. | PCDH19 RNA (% UTC) |
|---|---|
| 1688359 | 26 |
| 1688389 | 61 |
| 1688404 | 71 |
| 1688431 | 45 |
| 1688440 | 43 |
| 1688443 | 46 |
| 1688464 | 25 |
| 1688467 | 35 |
| 1688494 | 34 |
| 1688521 | 17 |
| 1688524 | 132 |
| 1688542 | 66 |
| 1688581 | 54 |
| 1688599 | 66 |
| 1688946 | 24 |
| 1688949 | 29 |
| 1688961 | 17 |
| 1688973 | 2† |
| 1688985 | 9† |
| 1689000 | 59 |
| 1689015 | 26 |
| 1689024 | 79 |
| 1689063 | 14 |
| 1689069 | 76 |
| 1689096 | 48 |
| 1689129 | 11 |
| 1689132 | 26 |
| 1689153 | 37 |
| 1689168 | 26 |
| 1689183 | 36 |
| 1689195 | 44 |
| 1689204 | 27 |
| 1689213 | 22 |
| 1689231 | 35 |
| 1689243 | 21 |
| 1689258 | 34 |
| 1689270 | 19 |
| 1689276 | 32 |
| 1689282 | 33 |
| 1689285 | 52 |
| 1689297 | 29 |
| 1689306 | 24 |
| 1689333 | 39 |
| 1689351 | 36 |
| 1689381 | 24 |
| 1689411 | 26 |
| 1689414 | 44 |
| 1689453 | 34 |
| 1689459 | 21 |
| 1689465 | 27 |
| 1689489 | 33 |
| 1689501 | 36 |
| 1689504 | 20 |
| 1689531 | 51 |
| 1689546 | 38 |
| 1689552 | 31 |
| 1689555 | 24 |
| 1689576 | 17 |
| 1689603 | 109 |
| 1689633 | 29 |
| 1689684 | 47 |
| 1689726 | 52 |
| 1689744 | 97 |
| 1689783 | 47 |
| 1689801 | 37 |
| 1689804 | 34 |
| 1689819 | 21 |
| 1689825 | 69 |
| 1689837 | 39 |
| 1689858 | 59 |
| 1689861 | 88 |
| 1689870 | 46 |
| 1689915 | 69 |
| 1689939 | 45 |
| 1690002 | 25 |
| 1690005 | 40 |
| 1690008 | 64 |
| 1690032 | 79 |

TABLE 14
Reduction of PCDH19 RNA by RNAi compounds at
a concentration of 200 nM in HEK293 cells

| Compound No. | PCDH19 RNA (% UTC) |
|---|---|
| 1688317 | 70 |
| 1688320 | 37 |
| 1688335 | 41 |
| 1688338 | 59 |
| 1688347 | 44 |
| 1688353 | 72 |
| 1688392 | 48 |
| 1688395 | 26 |
| 1688407 | 31 |
| 1688425 | 18 |
| 1688434 | 32 |
| 1688446 | 77 |
| 1688476 | 65 |
| 1688485 | 22 |
| 1688509 | 33 |
| 1688578 | 65 |
| 1688584 | 78 |
| 1688596 | 62 |
| 1688614 | 12 |
| 1688620 | 45 |
| 1688958 | 95 |
| 1688967 | 15 |
| 1688979 | 10† |
| 1688991 | 7† |
| 1689033 | 50 |
| 1689042 | 69 |
| 1689057 | 21 |
| 1689087 | 13 |
| 1689105 | 10 |
| 1689123 | 41 |
| 1689138 | 20 |
| 1689159 | 23 |
| 1689174 | 31 |
| 1689273 | 32 |
| 1689288 | 24 |
| 1689309 | 32 |
| 1689312 | 36 |
| 1689315 | 18 |
| 1689321 | 39 |
| 1689327 | 92 |
| 1689339 | 45 |
| 1689354 | 31 |
| 1689363 | 47 |
| 1689366 | 36 |
| 1689378 | 31 |
| 1689432 | 24 |
| 1689468 | 25 |
| 1689480 | 19 |
| 1689528 | 24 |
| 1689534 | 111 |
| 1689564 | 36 |
| 1689600 | 41 |
| 1689624 | 45 |
| 1689627 | 29 |
| 1689645 | 62 |
| 1689648 | 247 |
| 1689660 | 37 |
| 1689663 | 43 |
| 1689705 | 86 |
| 1689711 | 38 |
| 1689723 | 31 |
| 1689732 | 33 |
| 1689765 | 29 |
| 1689777 | 45 |
| 1689789 | 54 |
| 1689798 | 23 |
| 1689822 | 16 |
| 1689834 | 28 |
| 1689843 | 70 |
| 1689927 | 32 |
| 1689957 | 37 |
| 1689972 | 27 |
| 1689975 | 16 |
| 1689981 | 39 |
| 1689990 | 73 |
| 1690011 | 34 |
| 1690017 | 33 |
| 1690023 | 170 |

TABLE 15
Reduction of PCDH19 RNA by RNAi compounds at
a concentration of 200 nM in HEK293 cells

| Compound No. | PCDH19 RNA (% UTC) |
|---|---|
| 1688344 | 37 |
| 1688380 | 107 |
| 1688386 | 47 |
| 1688437 | 91 |
| 1688461 | 76 |
| 1688470 | 39 |
| 1688479 | 51 |
| 1688488 | 97 |
| 1688491 | 19 |
| 1688500 | 32 |
| 1688515 | 52 |
| 1688518 | 51 |
| 1688539 | 46 |
| 1688554 | 32 |
| 1688569 | 43 |
| 1688587 | 74 |
| 1688605 | 47 |
| 1688611 | 22 |
| 1688623 | 82 |
| 1688970 | 53† |
| 1688994 | 17† |
| 1689036 | 38 |
| 1689075 | 32 |
| 1689144 | 43 |
| 1689165 | 21 |
| 1689192 | 24 |
| 1689210 | 19 |
| 1689222 | 15 |
| 1689249 | 32 |
| 1689252 | 75 |
| 1689291 | 24 |
| 1689294 | 23 |
| 1689303 | 27 |
| 1689324 | 36 |
| 1689342 | 20 |
| 1689360 | 34 |
| 1689369 | 17 |
| 1689375 | 25 |
| 1689393 | 26 |
| 1689408 | 40 |
| 1689423 | 30 |
| 1689438 | 36 |
| 1689441 | 45 |
| 1689456 | 33 |
| 1689462 | 37 |
| 1689486 | 51 |
| 1689498 | 22 |
| 1689543 | 30 |
| 1689561 | 26 |
| 1689579 | 95 |
| 1689597 | 51 |
| 1689609 | 51 |
| 1689618 | 20 |
| 1689630 | 42 |
| 1689636 | 45 |
| 1689675 | 42 |
| 1689714 | 44 |
| 1689720 | 90 |
| 1689735 | 41 |
| 1689762 | 59 |
| 1689768 | 26 |
| 1689771 | 128 |
| 1689816 | 38 |
| 1689831 | 31 |
| 1689852 | 51 |
| 1689891 | 41 |
| 1689894 | 147 |
| 1689897 | 54 |
| 1689909 | 44 |
| 1689912 | 49 |
| 1689936 | 53 |
| 1689951 | 47 |
| 1689960 | 32 |
| 1689966 | 24 |
| 1689993 | 47 |
| 1689996 | 38 |
| 1689999 | 37 |
| 1690029 | 85 |

TABLE 16
Reduction of PCDH19 RNA by RNAi compounds at
a concentration of 200 nM in HEK293 cells

| Compound No. | PCDH19 RNA (% UTC) |
|---|---|
| 1688314 | 38 |
| 1688323 | 54 |
| 1688329 | 44 |
| 1688350 | 60 |
| 1688356 | 30 |
| 1688365 | 26 |
| 1688368 | 43 |
| 1688383 | 35 |
| 1688401 | 15 |
| 1688410 | 24 |
| 1688413 | 45 |
| 1688416 | 38 |
| 1688428 | 81 |
| 1688449 | 26 |
| 1688473 | 18 |
| 1688503 | 53 |
| 1688545 | 18 |
| 1688548 | 26 |
| 1688560 | 51 |
| 1689003 | 31 |
| 1689018 | 75 |
| 1689021 | 22 |
| 1689027 | 52 |
| 1689030 | 45 |
| 1689048 | 37 |
| 1689054 | 24 |
| 1689078 | 17 |
| 1689081 | 17 |
| 1689099 | 30 |
| 1689108 | 33 |
| 1689111 | 20 |
| 1689114 | 50 |
| 1689147 | 18 |
| 1689162 | 21 |
| 1689171 | 31 |
| 1689177 | 21 |
| 1689180 | 23 |
| 1689216 | 39 |
| 1689237 | 18 |
| 1689264 | 53 |
| 1689279 | 40 |
| 1689300 | 55 |
| 1689318 | 46 |
| 1689336 | 42 |
| 1689348 | 31 |
| 1689357 | 33 |
| 1689387 | 29 |
| 1689390 | 36 |
| 1689426 | 47 |
| 1689444 | 39 |
| 1689447 | 27 |
| 1689483 | 30 |
| 1689549 | 30 |
| 1689570 | 42 |
| 1689582 | 74 |
| 1689612 | 39 |
| 1689615 | 39 |
| 1689639 | 42 |
| 1689654 | 79 |
| 1689699 | 57 |
| 1689702 | 23 |
| 1689747 | 80 |
| 1689753 | 35 |
| 1689774 | 39 |
| 1689810 | 23 |
| 1689828 | 50 |
| 1689849 | 54 |
| 1689855 | 57 |
| 1689864 | 35 |
| 1689867 | 94 |
| 1689888 | 87 |
| 1689900 | 25 |
| 1689918 | 36 |
| 1689924 | 50 |
| 1689948 | 22 |
| 1689963 | 36 |
| 1689978 | 35 |
| 1689984 | 30 |

TABLE 17
Reduction of PCDH19 RNA by RNAi compounds at
a concentration of 200 nM in HEK293 cells

| Compound No. | PCDH19 RNA (% UTC) |
|---|---|
| 1688326 | 36 |
| 1688332 | 45 |
| 1688341 | 118 |
| 1688371 | 85 |
| 1688377 | 58 |
| 1688419 | 45 |
| 1688422 | 61 |
| 1688452 | 72 |
| 1688455 | 51 |
| 1688497 | 25 |
| 1688512 | 25 |
| 1688530 | 38 |
| 1688551 | 65 |
| 1688566 | 14 |
| 1688572 | 18 |
| 1688602 | 32 |
| 1688608 | 28 |
| 1688617 | 5 |
| 1688626 | 25 |
| 1688955 | 34 |
| 1688964 | 49 |
| 1688988 | 1† |
| 1688997 | 11† |
| 1689006 | 76 |
| 1689012 | 51 |
| 1689039 | 26 |
| 1689060 | 18 |
| 1689072 | 13 |
| 1689090 | 14 |
| 1689093 | 54 |
| 1689117 | 54 |
| 1689120 | 24 |
| 1689126 | 21 |
| 1689141 | 21 |
| 1689186 | 35 |
| 1689198 | 32 |
| 1689201 | 29 |
| 1689228 | 16 |
| 1689234 | 26 |
| 1689255 | 43 |
| 1689267 | 14 |
| 1689345 | 25 |
| 1689396 | 41 |
| 1689399 | 24 |
| 1689402 | 33 |
| 1689405 | 54 |
| 1689435 | 13 |
| 1689450 | 30 |
| 1689471 | 42 |
| 1689477 | 46 |
| 1689492 | 56 |
| 1689495 | 33 |
| 1689507 | 45 |
| 1689510 | 11 |
| 1689513 | 45 |
| 1689558 | 30 |
| 1689585 | 61 |
| 1689606 | 27 |
| 1689621 | 54 |
| 1689657 | 82 |
| 1689678 | 25 |
| 1689693 | 38 |
| 1689696 | 32 |
| 1689717 | 47 |
| 1689756 | 65 |
| 1689759 | 37 |
| 1689780 | 53 |
| 1689840 | 32 |
| 1689873 | 59 |
| 1689876 | 33 |
| 1689882 | 39 |
| 1689885 | 68 |
| 1689903 | 25 |
| 1689906 | 36 |
| 1689933 | 52 |
| 1689942 | 50 |
| 1689954 | 46 |
| 1689987 | 60 |

TABLE 18
Reduction of PCDH19 RNA by RNAi compounds at
a concentration of 200 nM in HEK293 cells

| Compound No. | PCDH19 RNA (% UTC) |
|---|---|
| 1688362 | 27 |
| 1688374 | 47 |
| 1688398 | 80 |
| 1688458 | 44 |
| 1688482 | 44 |
| 1688506 | 37 |
| 1688527 | 129 |
| 1688533 | 40 |
| 1688536 | 44 |
| 1688557 | 31 |
| 1688563 | 26 |
| 1688575 | 114 |
| 1688590 | 86 |
| 1688593 | 126 |
| 1688952 | 22 |
| 1688976 | 3† |
| 1688982 | 0† |
| 1689009 | 38 |
| 1689045 | 12 |
| 1689051 | 51 |
| 1689066 | 77 |
| 1689084 | 21 |
| 1689102 | 32 |
| 1689135 | 18 |
| 1689150 | 21 |
| 1689156 | 25 |
| 1689189 | 16 |
| 1689207 | 13 |
| 1689219 | 15 |
| 1689225 | 45 |
| 1689240 | 21 |
| 1689246 | 32 |
| 1689261 | 15 |
| 1689330 | 33 |
| 1689372 | 28 |
| 1689384 | 42 |
| 1689417 | 45 |
| 1689420 | 26 |
| 1689429 | 57 |
| 1689474 | 63 |
| 1689516 | 33 |
| 1689519 | 21 |
| 1689522 | 22 |
| 1689525 | 29 |
| 1689537 | 61 |
| 1689540 | 33 |
| 1689567 | 26 |
| 1689573 | 32 |
| 1689588 | 35 |
| 1689591 | 27 |
| 1689594 | 27 |
| 1689642 | 50 |
| 1689651 | 48 |
| 1689666 | 69 |
| 1689669 | 31 |
| 1689672 | 47 |
| 1689681 | 34 |
| 1689687 | 37 |
| 1689690 | 81 |
| 1689708 | 29 |
| 1689729 | 65 |
| 1689738 | 43 |
| 1689741 | 34 |
| 1689750 | 15 |
| 1689786 | 48 |
| 1689792 | 74 |
| 1689795 | 38 |
| 1689807 | 19 |
| 1689813 | 27 |
| 1689846 | 19 |
| 1689879 | 34 |
| 1689921 | 38 |
| 1689930 | 36 |
| 1689945 | 33 |
| 1689969 | 24 |
| 1690014 | 105 |
| 1690020 | 31 |
| 1690026 | 67 |

1.-6. (canceled)

7. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases complementary to:
an equal length portion of nucleobases 4,743-4,767 of SEQ ID NO: 1;
wherein the modified oligonucleotide comprises at least one modification selected from a modified sugar moiety and a modified internucleoside linkage.

8. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases of a sequence selected from:
SEQ ID NO: 132, 228, 284, 330, or 440;
wherein the modified oligonucleotide comprises at least one modification selected from a modified sugar moiety and a modified internucleoside linkage.

9. The oligomeric compound of claim 7, wherein the nucleobase sequence of the modified oligonucleotide is at least 80%, 85%, 90%, 95%, or 100% complementary to any of the nucleobase sequences of SEQ ID NO: 1 or SEQ ID NO: 2 when measured across the entire nucleobase sequence of the modified oligonucleotide.

10. The oligomeric compound of claim 7, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

11. (canceled)

12. (canceled)

13. The oligomeric compound of claim 7, wherein the modified sugar moiety comprises a bicyclic sugar moiety.

14. The oligomeric compound of claim 13, wherein the bicyclic sugar moiety comprises a 2′-4′ bridge selected from —O—CH₂—; and —O—CH(CH₃)—.

15. The oligomeric compound of claim 7, wherein the modified sugar moiety comprises a non-bicyclic modified sugar moiety.

16. (canceled)

17. The oligomeric compound of claim 15, wherein the non-bicyclic modified sugar moiety is a 2′-O(CH₂)₂—OCH₃ ribosyl sugar moiety, a 2′-OMe sugar moiety, or a 2′-F sugar moiety.

18. The oligomeric compound of claim 7, wherein the modified sugar moiety comprises a sugar surrogate.

19. (canceled)

20. (canceled)

21. (canceled)

22. The oligomeric compound of claim 7, wherein at least one internucleoside linkage is a phosphorothioate internucleoside linkage.

23. The oligomeric compound of claim 7, wherein the modified oligonucleotide comprises at least one phosphodiester internucleoside linkage.

24. The oligomeric compound of claim 7, wherein each internucleoside linkage is independently selected from a phosphodiester internucleoside linkage or a phosphorothioate internucleoside linkage.

25.-37. (canceled)

38. The oligomeric compound of claim 7, wherein the modified oligonucleotide is a gapmer.

39.-47. (canceled)

48. A population of oligomeric compounds of claim 7, wherein all of the phosphorothioate internucleoside linkages of the modified oligonucleotide are stereorandom.

49. An oligomeric duplex, comprising a first oligomeric compound and a second oligomeric compound comprising a second modified oligonucleotide, wherein the first oligomeric compound is the oligomeric compound of claim 7.

50.-88. (canceled)

89. A pharmaceutical composition comprising an oligomeric compound of claim 7, and a pharmaceutically acceptable diluent.

90. The pharmaceutical composition of claim 89, wherein the pharmaceutically acceptable diluent is artificial cerebrospinal fluid (aCSF) or PBS.

91. The pharmaceutical composition of claim 90, wherein the pharmaceutical composition consists essentially of the oligomeric compound and aCSF.

92. The pharmaceutical composition of claim 90, wherein the pharmaceutical composition consists essentially of the oligomeric compound and PBS.

93.-95. (canceled)

96. A method of treating a disease associated with PCDH19 comprising administering to a subject having or at risk for developing a disease associated with PCDH19 a therapeutically effective amount of an oligomeric compound of claim 7; and thereby treating the disease associated with PCDH19.

97.-109. (canceled)
